(19) United States
(12) Patent Application Publication
(10) Pub. No.: US 2009/0131342 A1
(43) Pub. Date: May 21, 2009

(54) Nitrosated and/or nitrosylated compounds, compositions and methods of use

(75) Inventor: James L. Ellis, Boxford, MA (US)

Correspondence Address: WILMERHALE/NITROMED, 1875 PENNSYLVANIA AVE, NW, WASHINGTON, DC, 20006, US

(73) Assignee: NitroMed, Inc., Lexington, MA (US)

(21) Appl. No.: 10/586,161

(22) PCT Filed: Jan. 24, 2005
(86) PCT No.: PCT/US05/02257

§ 371 Date: Jul. 14, 2006

Related U.S. Application Data
(60) Provisional application No. 60/537,918, filed on Jan. 22, 2004.

(51) Int. Cl. A61K 31/7048; C07H 19/10; A61P 31/04; A61K 31/7072

(52) U.S. Cl. 514/29; 536/26.26; 514/48

(57) ABSTRACT

The invention describes novel nitrosated and/or nitrosylated compounds of the invention and pharmaceutically acceptable salts thereof, and novel compositions comprising at least one nitrosated and/or nitrosylated compound of the invention, and, optionally, at least one nitric oxide donor and/or at least one therapeutic agent. The invention also provides novel compositions comprising at least one compound of the invention, and at least one nitric oxide donor and/or at least one therapeutic agent. The invention also provides novel kits comprising at least one compound of the invention, that is optionally nitrosated and/or nitrosylated, and, optionally, at least one nitric oxide donor and/or at least one therapeutic agent. The invention also provides methods for (a) treating bacterial infections; (b) treating viral infections; (c) treating fungal infections; and (d) treating lesions. The nitrosated and/or nitrosylated compounds of the invention are preferably nitrosated and/or nitrosylated antimicrobial compounds, nitrosated and/or nitrosylated adenosine antagonists, nitrosated and/or nitrosylated LTB4 antagonists, nitrosated and/or nitrosylated mucoregulators and nitrosated and/or nitrosylated purine agonists. The methods of the invention are preferably for the treatment of bacterial infections associated with pulmonary diseases such as cystic fibrosis.

### RELATED APPLICATIONS

This application claims priority under 35 USC § 119 to U.S. Application No. 60/537,918 filed Jan. 22, 2004.

### FIELD OF THE INVENTION

The invention describes novel nitrosated and/or nitrosylated compounds of the invention and pharmaceutically acceptable salts thereof, and novel compositions comprising at least one nitrosated and/or nitrosylated compound of the invention, and, optionally, at least one nitric oxide donor and/or at least one therapeutic agent. The invention also provides novel compositions comprising at least one compound of the invention, and at least one nitric oxide donor and/or at least one therapeutic agent. The invention also provides novel kits comprising at least one compound of the invention, that is optionally nitrosated and/or nitrosylated, and, optionally, at least one nitric oxide donor and/or at least one therapeutic agent. The invention also provides methods for (a) treating bacterial infections; (b) treating viral infections; (c) treating fungal infections; and (d) treating lesions. The nitrosated and/or nitrosylated compounds of the invention are preferably nitrosated and/or nitrosylated antimicrobial compounds, nitrosated and/or nitrosylated adenosine antagonists, nitrosated and/or nitrosylated LTB4 antagonists, nitrosated and/or nitrosylated mucoregulators and nitrosated and/or nitrosylated purine agonists. The methods of the invention are preferably for the treatment of bacterial infections associated with pulmonary diseases such as cystic fibrosis.

### BACKGROUND OF THE INVENTION

Antimicrobial compounds are used to control infections, to treat life-threatening diseases and to reduce death and illness. However, many antimicrobial compounds and antiviral compounds are potent anti-infective agents and also cause toxic side-effects such as skin rashes, shock and other allergic responses, toxic effects on the stomach, liver and kidney. In addition the wide use of antimicrobial compounds and antiviral compounds in the treatment of infections has caused the development of strains resistant to these drugs.

Hence there is a need in the art for antimicrobial compounds that can be administered to treat infections and that have improved efficacy, lower toxicity, can be used at low dosages and reduce microbial resistance. The invention is directed to these, as well as other, important ends.

### SUMMARY OF THE INVENTION

The invention provides novel compounds that are substituted with at least one NO and/or NO₂ group (i.e., nitrosylated and/or nitrosated), and pharmaceutically acceptable salts thereof. The compounds of the invention can be, for example, antimicrobial compounds, mucoregulators, purine agonists, LTB4 antagonists and adenosine antagonists. The compounds can be nitrosated and/or nitrosylated through one or more sites such as oxygen (hydroxyl condensation), sulfur (sulfhydryl condensation) and/or nitrogen. The invention also provides compositions comprising the novel compounds described herein in a pharmaceutically acceptable carrier.

The invention is also based on the discovery that administering at least one compound of the invention or a pharmaceutically acceptable salt thereof, that is optionally substituted with at least one NO and/or NO₂ group (i.e., nitrosylated and/or nitrosated), and, optionally, at least one nitric oxide donor improves the properties of the compound. Nitric oxide donors include, for example, S-nitrosothiols, nitrites, nitrates, N-oxo-N-nitrosamines, SPM 3672, SPM 5185, SPM 5186 and analogues thereof, and substrates of the various isozymes of nitric oxide synthase. Thus, another embodiment of the invention provides compositions comprising at least one compound of the invention, that is optionally substituted with at least one NO and/or NO₂ group (i.e., nitrosylated and/or nitrosated), and at least one nitric oxide donor compound. The invention also provides for such compositions in a pharmaceutically acceptable carrier.

Another embodiment of the invention provides compositions comprising at least one compound of the invention, that is optionally substituted with at least one NO and/or NO₂ group (i.e., nitrosylated and/or nitrosated), and, optionally, at least one nitric oxide donor compound and/or at least one therapeutic agent, including, but not limited to, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and combinations of two or more thereof. In a preferred embodiment the at least one therapeutic agent is selected from the group consisting of a β-adrenergic agonist, an anti-allergic compound, an antitussive compound, an antioxidant, a bronchodilator, an expectorant, a H₂ receptor antagonist, a nonsteroidal antiinflammatory compound (NSAIDs), a phosphodiesterase inhibitor, a proton pump inhibitor, a selective cyclooxygenase-2 (COX-2) inhibitor and a steroid. The invention also provides for such compositions in a pharmaceutically acceptable carrier.

Yet another embodiment of the invention provides methods for (a) treating bacterial infections; (b) treating viral infections; (c) treating fungal infections; and (d) treating lesions in a patient in need thereof comprising administering to the patient a therapeutically effective amount of at least one compound of the invention, that is optionally substituted with at least one NO and/or NO₂ group (i.e., nitrosylated and/or nitrosated), and, optionally, at least one nitric oxide donor compound. The methods can optionally further comprise the administration of at least one therapeutic agent, such as, for example, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and combinations of two or more thereof. In this embodiment of the invention, the methods can involve (i) administering the nitrosated and/or nitrosylated compounds of the invention, (ii) administering the compounds of the invention, that are optionally nitrosated and/or nitrosylated, and NO donors, (iii) administering the compounds of the invention, that are optionally nitrosated and/or nitrosylated, and therapeutic agents, or (iv) administering the compounds of the invention, that are optionally nitrosated and/or nitrosylated, NO donor compounds, and therapeutic agents. The compounds of the invention, nitric oxide donors, and/or therapeutic agents can be administered separately or as components of the same composition in one or more pharmaceutically acceptable carriers.

Another embodiment of the invention provides kits comprising at least one compound of the invention, that is optionally nitrosated and/or nitrosylated, and, optionally, at least one nitric oxide donor compound. The kit can further comprise at least one therapeutic agent, such as, for example, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and combinations of two or more thereof. The compound of the invention, the nitric oxide donor and/or therapeutic agent, can be separate components in the kit or can be in the form of a composition in one or more pharmaceutically acceptable carriers.

These and other aspects of the invention are described in detail herein.

### DETAILED DESCRIPTION OF THE INVENTION

As used throughout the disclosure, the following terms, unless otherwise indicated, shall be understood to have the following meanings.

“Compound” or “compound of the invention” refers to a non-nitrosated and/or non-nitrosylated compound of the invention, or pharmaceutically acceptable salts thereof or pharmaceutically acceptable esters thereof. “Compound” or “compound of the invention” includes the antimicrobial compounds, adenosine antagonists, LTB4 antagonists, mucoregulators and purine agonists, before they are nitrosated and/or nitrosylated by the methods described herein.

“Antimicrobial compound” refers to any compound that alters the growth of bacterial, fungi or virus cells whereby growth is prevented, modified, impaired, stabilized, inhibited or terminated. Antimicrobial compounds can be microbiocidal or microbiostatic and include, but are not limited to antibiotics, chemotherapeutic agents, semisynthetic antibiotics, synthetic antibiotics, antifungal compounds, antiviral compounds, and the like.

“Antifungal compound” refers to any compound that alters the growth of fungi whereby growth is prevented, modified, impaired, stabilized, inhibited or terminated.

“Antiviral compound” refers to any compound that alters the growth of viral cells whereby growth is prevented, modified, impaired, stabilized, inhibited or terminated.

“Bacterial infection” refers to any infection resulting from a bacteria or pathogen, including but not limited to infections resulting from Acinetobacter, Actinomyces israelii, Alcaligenes xylosoxidans, Bacillus anthracis, Borrelia burgdorferi, Borrelia recurrentis, Brucella, Burkolderia cepacia, Campylobacter jejumi, Campylobacter fetus, Calymmatobacterium granulomatis, Chlamydia psittaci, Chlamydia pneumoniae, Chlamydia trachomatis, Clostridium perfringens, Clostridium tetani, Clostridium difficilee, Corynebacterium diphtheriae, Corynebacterium species, Enterobacter species, Erysipelothris rhusiopathiae, Escherichia coli, Flavobacterium meningosepticum, Francisella tularensis, Fusobacterium nucleatum, Haemophilus ducreyi, Haemophilus influenzae, Klebsiella pneumoniae, Klebsiella oxytoca, Legionella pneumophila, Leptospira, Listeria monocytogenes, Moraxella catarrhalis, Mycobacterium avium-intracellulare, Mycobacterium tuberculosis, Mycobacterium leprae, Mycoplasma pneumoniae, Neisseria gonorrhoeae, Neisseria meningitides, Nitrobacter species, Nocardia asterodies, Pasteurella multocida, Pneumocystis carinii, Proteus mirabilis, Proteus, Pseudomonas aeruginosa, Pseudomonas mallei, Pseudomonas pseudomallei, Ricckettsia, Salmonella, Shigella, Serratia, Streptococcus. aureus, Streptococcus pneumoniae, Streptococcus pyrigens, Streptococcus, Streptococcus agalactiae, Streptococcus bovis, Streptobacillus moniliformis, Serratia marcescens, Stenotrophomonas maltophilia, Treponema pallidum, Treponema pertenue, Ureaplasma urealyticum, Vibria cholerae, Yersinia pestis, Yersinia enterocolitica, and the like; pulmonary infections in patients with disease including, but not limited to, endobronchial infections, cystic fibrosis, bronchiectasis, pneumonia, tuberculosis, emphysema, AIDS, pneumoccal meningitis, bacteremia, otitis media, chronic obstructive pulmonary disease, sinus congestion, common cold, septicemia and the like; gastrointestinal infections, including, but not limited to, chronic gastritis, gastric ulcer, duodenal ulcer, Helicobacter pylori, gastric malignant lymphoma, gastroenteritis, diarrhea, dysentery, inflammatory bowel disease, Crohn's disease, ulcerative colitis, infections resulting from E., coli, and the like; and infections of the eyes, ear or nose.

“Fungal infection” refers to and includes any infection resulting from a fungi, including but not limited to, infections resulting from Aspergillus species, agents of mucormycosis, Blastomyces dermatitidis, Candida species, Coccidiodes immnitis, Cryptococcus neoformans, Histoplasma capsulatum, Mucoramycosis pseudallescheriasis, Paracoccidiodies brasiliensis, Sporothris schenckii, and the like.

“Viral infection” refers to and includes any infection resulting from a virus, including but not limited to infections resulting from adenovirus, anaerobic bacilli, cytomegalovirus, corona virus, cellulites, Epstein barr virus, Herpes simplex virus, human immunodeficiency virus (HIV), human papilloma virus, influenza virus, mycobacteria, parainfluenza virus, picornavirus, papilloma virus, respiratory syncytial virus, staphylococci, streptococci, synsytial virus, varicella zostar virus, severe acute respiratory syndrome (SARS) and the like. Microbial infection includes dental diseases such as gingival inflammations, periodontal inflammations, dental caries, and the like.

“Lesion” refers to and includes any lesion such as those caused by antineoplactic therapy such as radiation, chemotherapy; surgical intervention such as hemorrhoidectomy, biopsy procedure, resection; herpes virus; lesions of the distal bowel such as proctitis, enteritis, Crohn's disease, ulcerative colitis, those resulting from microbial infections, and the like.

“Therapeutic agent” includes any therapeutic agent that can be used to treat or prevent the diseases described herein. “Therapeutic agents” include, for example, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and the like. Therapeutic agent includes the prodrugs and pharmaceutical derivatives thereof including, but not limited to, the corresponding nitrosated and/or nitrosylated derivatives. Although nitric oxide donors have, therapeutic activity, the term “therapeutic agent” does not include the nitric oxide donors described herein, since nitric oxide donors are separately defined.

“Prodrug” refers to a compound that is made more active in vivo.

“Antioxidant” refers to and includes any compound that can react and quench a free radical.

“Angiotensin converting enzyme (ACE) inhibitor” refers to compounds that inhibit an enzyme which catalyzes the conversion of angiotensin I to angiotensin II. ACE inhibitors include, but are not limited to, amino acids and derivatives thereof, peptides, including di- and tri-peptides, and antibodies to ACE which intervene in the renin-angiotensin system by inhibiting the activity of ACE thereby reducing or eliminating the formation of the pressor substance angiotensin II.

“Angiotensin II antagonists” refers to compounds which interfere with the function, synthesis or catabolism of angiotensin II. Angiotensin II antagonists include peptide compounds and non-peptide compounds, including, but not limited to, angiotensin II antagonists, angiotensin II receptor antagonists, agents that activate the catabolism of angiotensin II, and agents that prevent the synthesis of angiotensin I from angiotensin II. The renin-angiotensin system is involved in the regulation of hemodynamics and water and electrolyte balance. Factors that lower blood volume, renal perfusion pressure, or the concentration of sodium in plasma tend to activate the system, while factors that increase these parameters tend to suppress its function.

“Anti-hyperlipidemic compounds” refers to any compound or agent that has the effect of beneficially modifying serum cholesterol levels such as, for example, lowering serum low density lipoprotein (LDL) cholesterol levels, or inhibiting oxidation of LDL cholesterol, whereas high density lipoprotein (HDL) serum cholesterol levels may be lowered, remain the same, or be increased. Preferably, the anti-hyperlipidemic compound brings the serum levels of LDL cholesterol and HDL cholesterol (and, more preferably, triglyceride levels) to normal or nearly normal levels.

“Diuretic compound” refers to and includes any compound or agent that increases the amount of urine excreted by a patient.

“Neutral endopeptidase inhibitors” refers to and includes compounds that are antagonists of the renin angiotensin aldosterone system including compounds that are dual inhibitors of neutral endopeptidases and angiotensin converting (ACE) enzymes.

“Renin inhibitors” refers to compounds which interfere with the activity of renin.

“Phosphodiesterase inhibitor” or “PDE inhibitor” refers to any compound that inhibits the enzyme phosphodiesterase. The term refers to selective or non-selective inhibitors of cyclic guanosine 3′,5′-monophosphate phosphodiesterases (cGMP-PDE) and cyclic adenosine 3′,5′-monophosphate phosphodiesterases (cAMP-PDE).

“Platelet reducing agents” refers to compounds that prevent the formation of a blood thrombus via any number of potential mechanisms. Platelet reducing agents include, but are not limited to, fibrinolytic agents, anti-coagulant agents and any inhibitors if platelet function. Inhibitors of platelet function include agents that impair the ability of mature platelets to perform their normal physiological roles (i.e., their normal function, such as, for example, adhesion to cellular and non-cellular entities, aggregation, release of factors such as growth factors) and the like.

“Proton pump inhibitor” refers to any compound that reversibly or irreversibly blocks gastric acid secretion by inhibiting the H⁺/K⁺-ATP ase enzyme system at the secretory surface of the gastric parietal cell.

“NSAID” refers to a nonsteroidal anti-inflammatory compound or a nonsteroidal anti-inflammatory drug. NSAIDs inhibit cyclooxygenase, the enzyme responsible for the biosyntheses of the prostaglandins and certain autocoid inhibitors, including inhibitors of the various isozymes of cyclooxygenase (including but not limited to cyclooxygenase-1 and -2), and as inhibitors of both cyclooxygenase and lipoxygenase.

“Cyclooxygenase-2 (COX-2) selective inhibitor” refers to a compound that selectively inhibits the cyclooxygenase-2 enzyme over the cyclooxygenase-1 enzyme. In one embodiment, the compound has a cyclooxygenase-2 IC₅₀ of less than about 2 μM and a cyclooxygenase-1 IC₅₀ of greater than about 5 μM, in the human whole blood COX-2 assay (as described in Brideau et al., Inflamm Res., 45: 68-74 (1996)) and also has a selectivity ratio of cyclooxygenase-2 inhibition over cyclooxygenase-1 inhibition of at least 10, and preferably of at least 40. In another embodiment, the compound has a cyclooxygenase-1 IC₅₀ of greater than about 1 μM, and preferably of greater than 20 μM. The compound can also inhibit the enzyme, lipoxygenase. Such selectivity may indicate an ability to reduce the incidence of common NSAID-induced side effects.

“Patient” refers to animals, preferably mammals, most preferably humans, and includes males and females, and children and adults.

“Therapeutically effective amount” refers to the amount of the compound and/or composition that is effective to achieve its intended purpose.

“Transdermal” refers to the delivery of a compound by passage through the skin and into the blood stream.

“Transmucosal” refers to delivery of a compound by passage of the compound through the mucosal tissue and into the blood stream.

“Inhaled” or “inhalation” refers to the delivery of a compound where a maximum amount of compound is delivered to the patient's airways, respiratory tract and/or lungs.

“Penetration enhancement” or “permeation enhancement” refers to an increase in the permeability of the skin or mucosal tissue to a selected pharmacologically active compound such that the rate at which the compound permeates through the skin or mucosal tissue is increased.

“Carriers” or “vehicles” refers to carrier materials suitable for compound administration and include any such material known in the art such as, for example, any liquid, gel, solvent, liquid diluent, solubilizer, or the like, which is non-toxic and which does not interact with any components of the composition in a deleterious manner.

“Sustained release” refers to the release of a therapeutically active compound and/or composition such that the blood levels of the therapeutically active compound are maintained within a desirable therapeutic range over a period of time. The sustained release formulation can be prepared using any conventional method known to one skilled in the art to obtain the desired release characteristics.

“Nitric oxide adduct” or “NO adduct” refers to compounds and functional groups which, under physiological conditions, can donate, release and/or directly or indirectly transfer any of the three redox forms of nitrogen monoxide (NO⁺, NO⁻, NO), such that the biological activity of the nitrogen monoxide species is expressed at the intended site of action.

“Nitric oxide releasing” or “nitric oxide donating” refers to methods of donating, releasing and/or directly or indirectly transferring any of the three redox forms of nitrogen monoxide (NO⁺, NO⁻, NO), such that the biological activity of the nitrogen monoxide species is expressed at the intended site of action.

“Nitric oxide donor” or “NO donor” refers to compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo and/or are oxidized to produce nitric oxide and/or are substrates for nitric oxide synthase and/or cytochrome P450. “NO donor” also includes compounds that are precursors of L-arginine, inhibitors of the enzyme arginase and nitric oxide mediators.

“Alkyl” refers to a lower alkyl group, a substituted lower alkyl group, a haloalkyl group, a hydroxyalkyl group, an alkenyl group, a substituted alkenyl group, an alkynyl group, a bridged cycloalkyl group, a cycloalkyl group or a heterocyclic ring, as defined herein. An alkyl group may also comprise one or more radical species, such as, for example a cycloalkylalkyl group or a heterocyclicalkyl group.

“Lower alkyl” refers to branched or straight chain acyclic alkyl group comprising one to about ten carbon atoms (preferably one to about eight carbon atoms, more preferably one to about six carbon atoms). Exemplary lower alkyl groups include methyl, ethyl, n-propyl, isopropyl, n-butyl, isobutyl, sec-butyl, t-butyl, pentyl, neopentyl, iso-amyl, hexyl, octyl, and the like.

“Substituted lower alkyl” refers to a lower alkyl group, as defined herein, wherein one or more of the hydrogen atoms have been replaced with one or more R¹⁰⁰ groups, wherein each R¹⁰⁰ is independently a hydroxy, an ester, an amidyl, an oxo, a carboxyl, a carboxamido, a halo, a cyano, a nitrate or an amino group, as defined herein.

“Haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein. Exemplary haloalkyl groups include trifluoromethyl, chloromethyl, 2-bromobutyl, 1-bromo-2-chloro-pentyl, and the like.

“Alkenyl” refers to a branched or straight chain C₂-C₁₀ hydrocarbon (preferably a C₂-C₈ hydrocarbon, more preferably a C₂-C₆ hydrocarbon) that can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like.

“Lower alkenyl” refers to a branched or straight chain C₂-C₄ hydrocarbon that can comprise one or two carbon-carbon double bonds.

“Substituted alkenyl” refers to a branched or straight chain C₂-C₁₀ hydrocarbon (preferably a C₂-C₈ hydrocarbon, more preferably a C₂-C₆ hydrocarbon) which can comprise one or more carbon-carbon double bonds, wherein one or more of the hydrogen atoms have been replaced with one or more R¹⁰⁰ groups, wherein each R¹⁰⁰ is independently a hydroxy, an oxo, a carboxyl, a carboxamido, a halo, a cyano or an amino group, as defined herein.

“Alkynyl” refers to an unsaturated acyclic C₂-C₁₀ hydrocarbon (preferably a C₂-C₈ hydrocarbon, more preferably a C₂-C₆ hydrocarbon) that can comprise one or more carbon-carbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimethyl-butyn-1-yl, and the like.

“Bridged cycloalkyl” refers to two or more cycloalkyl groups, heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo(3,3,0)octane, 7-oxabicyclo(2,2,1)heptyl, 8-azabicyclo(3,2,1)oct-2-enyl and the like.

“Cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo, alkylsulfinyl, and nitro. Exemplary cycloalkyl groups include cyclopropyl, cyclobutyl, cyclopentyl, cyclohexyl, cyclohexenyl, cyclohepta-1,3-dienyl, and the like.

“Heterocyclic ring or group” refers to a saturated or unsaturated cyclic hydrocarbon group having about 2 to about 10 carbon atoms (preferably about 4 to about 6 carbon atoms) where 1 to about 4 carbon atoms are replaced by one or more nitrogen, oxygen and/or sulfur atoms. Sulfur may be in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, alkylthio, aryloxy, arylthio, arylalkyl, hydroxy, oxo, thial, halo, carboxyl, carboxylic ester, alkylcarboxylic acid, alkylcarboxylic ester, aryl, arylcarboxylic acid, arylcarboxylic ester, amidyl, ester, alkylcarbonyl, arylcarbonyl, alkylsulfinyl, carboxamido, alkylcarboxamido, arylcarboxamido, sulfonic acid, sulfonic ester, sulfonamide nitrate and nitro. Exemplary heterocyclic groups include pyrrolyl, furyl, thienyl, 3-pyrrolinyl,4,5,6-trihydro-2H-pyranyl, pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrahydrofuranyl, tetrazolyl, pyrrolinyl, pyrrolindinyl, oxazolindinyl 1,3-dioxolanyl, imidazolinyl, imidazolindinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, benzothiazolinyl, quinolinyl, 2,6-dioxabicyclo(3,3,0)octane, and the like.

“Heterocyclic compounds” refer to mono- and polycyclic compounds comprising at least one aryl or heterocyclic ring.

“Aryl” refers to a monocyclic, bicyclic, carbocyclic or heterocyclic ring system comprising one or two aromatic rings. Exemplary aryl groups include phenyl, pyridyl, napthyl, quinoyl, tetrahydronaphthyl, furanyl, indanyl, indenyl, indoyl, and the like. Aryl groups (including bicyclic aryl groups) can be unsubstituted or substituted with one, two or three substituents independently selected from alkyl, alkoxy, alkylthio, amino, alkylamino, dialkylamino, arylamino, diarylamino, alkylarylamino, halo, cyano, alkylsulfinyl, hydroxy, carboxyl, carboxylic ester, alkylcarboxylic acid, alkylcarboxylic ester, aryl, arylcarboxylic acid, arylcarboxylic ester, alkylcarbonyl, arylcarbonyl, amidyl, ester, carboxamido, alkylcarboxamido, carbomyl, sulfonic acid, sulfonic ester, sulfonamido and nitro. Exemplary substituted aryl groups include tetrafluorophenyl, pentafluorophenyl, sulfonamide, alkylsulfonyl, arylsulfonyl, and the like.

“Cycloalkenyl” refers to an unsaturated cyclic C₂-C₁₀ hydrocarbon (preferably a C₂-C₈ hydrocarbon, more preferably a C₂-C₆ hydrocarbon) which can comprise one or more carbon-carbon triple bonds.

“Alkylaryl” refers to an alkyl group, as defined herein, to which is appended an aryl group, as defined herein. Exemplary alkylaryl groups include benzyl, phenylethyl, hydroxybenzyl, fluorobenzyl, fluorophenylethyl, and the like.

“Arylalkyl” refers to an aryl radical, as defined herein, attached to an alkyl radical, as defined herein. Exemplary arylalkyl groups include benzyl, phenylethyl, 4-hydroxybenzyl, 3-fluorobenzyl, 2-fluorophenylethyl, and the like.

“Arylalkenyl” refers to an aryl radical, as defined herein, attached to an alkenyl radical, as defined herein. Exemplary arylalkenyl groups include styryl, propenylphenyl, and the like.

“Cycloalkylalkyl” refers to a cycloalkyl radical, as defined herein, attached to an alkyl radical, as defined herein.

“Cycloalkylalkoxy” refers to a cycloalkyl radical, as defined herein, attached to an alkoxy radical, as defined herein.

“Cycloalkylalkylthio” refers to a cycloalkyl radical, as defined herein, attached to an alkylthio radical, as defined herein.

“Heterocyclicalkyl” refers to a heterocyclic ring radical, as defined herein, attached to an alkyl radical, as defined herein.

“Arylheterocyclic ring” refers to a bi- or tricyclic ring comprised of an aryl ring, as defined herein, appended via two adjacent carbon atoms of the aryl ring to a heterocyclic ring, as defined herein. Exemplary arylheterocyclic rings include dihydroindole, 1,2,3,4-tetra-hydroquinoline, and the like.

“Alkylheterocyclic ring” refers to a heterocyclic ring radical, as defined herein, attached to an alkyl radical, as defined herein. Exemplary alkylheterocyclic rings include 2-pyridylmethyl, 1-methylpiperidin-2-one-3-methyl, and the like.

“Alkoxy” refers to R₅₀O—, wherein R₅₀ is an alkyl group, as defined herein (preferably a lower alkyl group or a haloalkyl group, as defined herein). Exemplary alkoxy groups include methoxy, ethoxy, t-butoxy, cyclopentyloxy, trifluoromethoxy, and the like.

“Aryloxy” refers to R₅₅O—, wherein R₅₅ is an aryl group, as defined herein. Exemplary arylkoxy groups include napthyloxy, quinolyloxy, isoquinolizinyloxy, and the like.

“Alkylthio” refers to R₅₀S—, wherein R₅₀ is an alkyl group, as defined herein.

“Lower alkylthio” refers to a lower alkyl group, as defined herein, appended to a thio group, as defined herein.

“Arylalkoxy” or “alkoxyaryl” refers to an alkoxy group, as defined herein, to which is appended an aryl group, as defined herein. Exemplary arylalkoxy groups include benzyloxy, phenylethoxy, chlorophenylethoxy, and the like.

“Arylalklythio” or refers to an alkylthio group, as defined herein, to which is appended an aryl group, as defined herein. Exemplary arylalklythio groups include benzylthio, phenylethylthio, chlorophenylethylthio, and the like.

“Arylalklythioalkyl” or refers to an arylalkylthio group, as defined herein, to which is appended an alkyl group, as defined herein. Exemplary arylalklythioalkyl groups include benzylthiomethyl, phenylethylthiomethyl, chlorophenylethylthioethyl, and the like.

“Alkylthioalkyl” or refers to an alkylthio group, as defined herein, to which is appended an alkyl group, as defined herein. Exemplary alkylthioalkyl groups include allylthiomethyl, ethylthiomethyl, trifluoroethylthiomethyl, and the like.

“Alkoxyalkyl” refers to an alkoxy group, as defined herein, appended to an alkyl group, as defined herein. Exemplary alkoxyalkyl groups include methoxymethyl, methoxyethyl, isopropoxymethyl, and the like.

“Alkoxyhaloalkyl” refers to an alkoxy group, as defined herein, appended to a haloalkyl group, as defined herein. Exemplary alkoxyhaloalkyl groups include 4-methoxy-2-chlorobutyl and the like.

“Cycloalkoxy” refers to R₅₄O—, wherein R₅₄ is a cycloalkyl group or a bridged cycloalkyl group, as defined herein. Exemplary cycloalkoxy groups include cyclopropyloxy, cyclopentyloxy, cyclohexyloxy, and the like.

“Cycloalkylthio” refers to R₅₄S—, wherein R₅₄ is a cycloalkyl group or a bridged cycloalkyl group, as defined herein. Exemplary cycloalkylthio groups include cyclopropylthio, cyclopentylthio, cyclohexylthio, and the like.

“Haloalkoxy” refers to an alkoxy group, as defined herein, in which one or more of the hydrogen atoms on the alkoxy group are substituted with halogens, as defined herein. Exemplary haloalkoxy groups include 1,1,1-trichloroethoxy, 2-bromobutoxy, and the like.

“Hydroxy” refers to —OH.

“Oxy” refers to —O—

“Oxo” refers to ═O.

“Oxylate” refers to —O⁻R₇₇⁺ wherein R₇₇ is an organic or inorganic cation.

“Thiol” refers to —SH.

“Thio” refers to —S—.

“Oxime” refers to ═N—OR₈₁ wherein R₈₁ is a hydrogen, an alkyl group, an aryl group, an alkylsulfonyl group, an arylsulfonyl group, a carboxylic ester, an alkylcarbonyl group, an arylcarbonyl group, a carboxamido group, an alkoxyalkyl group or an alkoxyaryl group.

“Hydrazone refers to ═N—N(R₈₁)(R′₈₁) wherein R′₈₁ is independently selected from R₈₁, and R₈₁ is as defined herein.

“Hydrazino” refers to H₂N—N(H)—.

“Organic cation” refers to a positively charged organic ion. Exemplary organic cations include alkyl substituted ammonium cations, and the like.

“Inorganic cation” refers to a positively charged metal ion. Exemplary inorganic cations include Group I metal cations such as for example, sodium, potassium, magnesium, calcium, and the like.

“Hydroxyalkyl” refers to a hydroxy group, as defined herein, appended to an alkyl group, as defined herein.

“Nitrate” refers to —O—NO₂.

“Nitrite” refers to —O—NO.

“Thionitrate” refers to —S—NO₂.

“Thionitrite” and “nitrosothiol” refer to —S—NO.

“Nitro” refers to the group —NO₂ and “nitrosated” refers to compounds that have been substituted therewith.

“Nitroso” refers to the group —NO and “nitrosylated” refers to compounds that have been substituted therewith.

“Nitrile” and “cyano” refer to —CN.

“Halogen” or “halo” refers to iodine (I), bromine (Br), chlorine (Cl), and/or fluorine (F).

“Amino” refers to —NH₂, an alkylamino group, a dialkylamino group, an arylamino group, a diarylamino group, an alkylarylamino group or a heterocyclic ring, as defined herein.

“Alkylamino” refers to R₅₀NH—, wherein R₅₀ is an alkyl group, as defined herein. Exemplary alkylamino groups include methylamino, ethylamino, butylamino, cyclohexylamino, and the like.

“Arylamino” refers to R₅₅NH—, wherein R₅₅ is an aryl group, as defined herein.

“Dialkylamino” refers to R₅₂R₅₃N—, wherein R₅₂ and R₅₃ are each independently an alkyl group, as defined herein. Exemplary dialkylamino groups include dimethylamino, diethylamino, methyl propargylamino, and the like.

“Diarylamino” refers to R₅₅R₆₀N—, wherein R₅₅ and R₆₀ are each independently an aryl group, as defined herein.

“Alkylarylamino or arylalkylamino” refers to R₅₂R₅₅N—, wherein R₅₂ is an alkyl group, as defined herein, and R₅₅ is an aryl group, as defined herein.

“Alkylarylalkylamino” refers to R₅₂R₇₉N—, wherein R₅₂ is an alkyl group, as defined herein, and R₇₉ is an arylalkyl group, as defined herein.

“Alkylcycloalkylamino” refers to R₅₂R₈₀N—, wherein R₅₂ is an alkyl group, as defined herein, and R₈₀ is an cycloalkyl group, as defined herein.

“Aminoalkyl” refers to an amino group, an alkylamino group, a dialkylamino group, an arylamino group, a diarylamino group, an alkylarylamino group or a heterocyclic ring, as defined herein, to which is appended an alkyl group, as defined herein. Exemplary aminoalkyl groups include dimethylaminopropyl, diphenylaminocyclopentyl, methylaminomethyl, and the like.

“Aminoaryl” refers to an aryl group to which is appended an alkylamino group, a arylamino group or an arylalkylamino group. Exemplary aminoaryl groups include anilino, N-methylanilino, N-benzylanilino, and the like.

“Thio” refers to —S—.

“Sulfinyl” refers to —S(O)—.

“Methanthial” refers to —C(S)—.

“Thial” refers to ═S.

“Sulfonyl” refers to —S(O)₂⁻.

“Sulfonic acid” refers to —S(O)₂OR₇₆, wherein R₇₆ is a hydrogen, an organic cation or an inorganic cation, as defined herein.

“Alkylsulfonic acid” refers to a sulfonic acid group, as defined herein, appended to an alkyl group, as defined herein.

“Arylsulfonic acid” refers to a sulfonic acid group, as defined herein, appended to an aryl group, as defined herein

“Sulfonic ester” refers to —S(O)₂OR₅₈, wherein R₅₈ is an alkyl group, an aryl group, or an aryl heterocyclic ring, as defined herein.

“Sulfonamido” refers to —S(O)₂—N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylsulfonamido” refers to a sulfonamido group, as defined herein, appended to an alkyl group, as defined herein.

“Arylsulfonamido” refers to a sulfonamido group, as defined herein, appended to an aryl group, as defined herein.

“Alkylthio” refers to R₅₀S—, wherein R₅₀ is an alkyl group, as defined herein (preferably a lower alkyl group, as defined herein).

“Arylthio” refers to R₅₅S—, wherein R₅₅ is an aryl group, as defined herein.

“Arylalkylthio” refers to an aryl group, as defined herein, appended to an alkylthio group, as defined herein.

“Alkylsulfinyl” refers to R₅₀—S(O)—, wherein R₅₀ is an alkyl group, as defined herein.

“Alkylsulfonyl” refers to R₅₀—S(O)₂—, wherein R₅₀ is an alkyl group, as defined herein.

“Alkylsulfonyloxy” refers to R₅₀—S(O)₂—O—, wherein R₅₀ is an alkyl group, as defined herein.

“Arylsulfinyl” refers to R₅₅—S(O)—, wherein R₅₅ is an aryl group, as defined herein.

“Arylsulfonyl” refers to R₅₅—S(O)₂—, wherein R₅₅ is an aryl group, as defined herein.

“Arylsulfonyloxy” refers to R₅₅—S(O)₂—O—, wherein R₅₅ is an aryl group, as defined herein.

“Amidyl” refers to R₅₁C(O)N(R₅₇)— wherein R₅₁ and R₅₇ are each independently a hydrogen atom, an alkyl group, an aryl group or an arylheterocyclic ring, as defined herein.

“Ester” refers to R₅₁C(O)R₇₆— wherein R₅₁ is a hydrogen atom, an alkyl group, an aryl group or an arylheterocyclic ring, as defined herein and R₇₆ is oxygen or sulfur.

“Carbamoyl” refers to —O—C(O)N(R₅₁)(R₅₇), wherein R₅₁ and R₅₇ are each independently a hydrogen atom, an alkyl group, an aryl group or an arylheterocyclic ring, as defined herein, or R₅₁ and R₅₇ taken together are a heterocyclic ring, a cycloalkyl group or a bridged cycloalkyl group, as defined herein.

“Carboxyl” refers to —C(O)OR₇₆, wherein R₇₆ is a hydrogen, an organic cation or an inorganic cation, as defined herein.

“Carbonyl” refers to —C(O)—.

“Alkylcarbonyl” refers to R₅₂—C(O)—, wherein R₅₂ is an alkyl group, as defined herein.

“Arylcarbonyl” refers to R₅₅—C(O)—, wherein R₅₅ is an aryl group, as defined herein.

“Arylalkylcarbonyl” refers to R₅₅-R₅₂—C(O)—, wherein R₅₅ is an aryl group, as defined herein, and R₅₂ is an alkyl group, as defined herein.

“Alkylarylcarbonyl” refers to R₅₂-R₅₅—C(O)—, wherein R₅₅ is an aryl group, as defined herein, and R₅₂ is an alkyl group, as defined herein.

“Heterocyclicalkylcarbonyl” refer to R₇₈C(O)— wherein R₇₈ is a heterocyclicalkyl group, as defined herein.

“Carboxylic ester” refers to —C(O)OR₅₈, wherein R₅₈ is an alkyl group, an aryl group or an aryl heterocyclic ring, as defined herein.

“Alkylcarboxylic acid” and “alkylcarboxyl” refer to an alkyl group, as defined herein, appended to a carboxyl group, as defined herein.

“Alkylcarboxylic ester” refers to an alkyl group, as defined herein, appended to a carboxylic ester group, as defined herein.

“Alkyl ester” refers to an alkyl group, as defined herein, appended to an ester group, as defined herein.

“Arylcarboxylic acid” refers to an aryl group, as defined herein, appended to a carboxyl group, as defined herein.

“Arylcarboxylic ester” and “arylcarboxyl” refer to an aryl group, as defined herein, appended to a carboxylic ester group, as defined herein.

“Aryl ester” refers to an aryl group, as defined herein, appended to an ester group, as defined herein.

“Carboxamido” refers to —C(O)N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylcarboxamido” refers to an alkyl group, as defined herein, appended to a carboxamido group, as defined herein.

“Arylcarboxamido” refers to an aryl group, as defined herein, appended to a carboxamido group, as defined herein.

“Urea” refers to —N(R₅₉)—C(O)N(R₅₁)(R₅₇) wherein R₅₁, R₅₇, and R₅₉ are each independently a hydrogen atom, an alkyl group, an aryl group or an arylheterocyclic ring, as defined herein, or R₅₁ and R₅₇ taken together are a heterocyclic ring, a cycloalkyl group or a bridged cycloalkyl group, as defined herein.

“Phosphoryl” refers to —P(R₇₀)(R₇₁)(R₇₂), wherein R₇₀ is a lone pair of electrons, thial or oxo, and R₇₁ and R₇₂ are each independently a covalent bond, a hydrogen, a lower alkyl, an alkoxy, an alkylamino, a hydroxy, an oxy or an aryl, as defined herein.

“Silyl” refers to —Si(R₇₃)(R₇₄)(R₇₅), wherein R₇₃, R₇₄ and R₇₅ are each independently a covalent bond, a lower alkyl, an alkoxy, an aryl or an arylalkoxy, as defined herein.

The compounds used in the compounds and compositions of the invention are preferably antimicrobial compounds, adenosine antagonists, LTB4 antagonists, mucoregulators and purine agonists.

Suitable antimicrobial compounds, include, but are not limited to, acediasulfone, aceturate, acetyl sulfametossipirazine, acetyl sulfamethoxypyrazine, acranil, albendazole, alexidine, amatadine, ambazone, amdinocillin, amikacin, p-aminosalicylic acid, p-aminosalicylic acid hydrazine, amoxicillin, ampicillin, anisomycin, apalcillin, apicyclin, apramycin, arbekacin, argininsa, aspoxicillin, azidamfenicol, azidocillin, azithromycin, azlocillin, aztreonam, bacampicillin, benzoylpas, benzyl penicillin acid, benzyl sulfamide, bicozamycin, bipenam, brodimoprim, capreomycim, carbenicillin, carbomycin, cafazedone, carindacillin, carumonam, cefcapene pivoxil, cefaclor, cefadroxil, cefafroxil, cefamandole, cefatamet, cefatrizine, cefazedone, cefazolin, cefbuperazone, cefclidin, cefdinir, cefditoren, cefixime, cefmenoxime, cefmetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftezole, ceftibuten, ceftiofur, ceftizoxime, ceftriaxone, cefuroxime, cefuzonam, cephacetrile sodium, cephadrine, cephalexin, cephaloglycin, cephaloridine, cephalosporin C, cephalothin, cephapirin sodium, cephradine, chloramphenicol, chlorotetracycline, cinoxacin, ciprofloxacin, claritromycin, clavulanic acid, clinafloxacin, clindamycin, clofazimine, clofoctal, clometocillin, clomocycline, cloxacillin, cloxyquin, cyclacilline, cycloserine, danoflaxcin, dapsone, deoxycycline, deoxydihydrostreptomycin, dibekacin, dicloxacillin, difloxacin, dihydrostreptomycin, dimetridazole, diminazene, dirirtomycin, eflornithine, enrofloxacin, enviomycin, epicillin, erythromycin, etacillin, ethambutol, ethionamide, famcyclovir, fenbecillin, fleroxacin, flomoxef, floxacillin, flumequine, furonazide, fortimycin, furazolium chloride, gentamycin, glyconiazide, grepafloxacin, guamecycline, halofuginone, hetacillin, homidium, hydroxyl-stilbamidine, ibostamycin, imidocarb, imipenam, ipronidazole, isoniazide, josamycin, inosine, lauroguadine, lenampicillin, lincomycin, lomefloxacin, loracarbef, lymecyclin, mafenide, mebendazole, meclocyclin, meropenem, metampicillin, metacicline, methacycline, methicillin sodium, metronidazole, 4′-(methylsulfamoyl)sulfanilanilide, mezlocillin, meziocillin, micronomycin, midecamycin A₁, minocycline, miocamycin, miokamycin, morfazinamide, moxalactam, mupirocin, myxin, nadifloxacin, nalidixic acid, negamycin, neomycin, netlimycin, nifurfoline, nifurpirinol, nifurprazine, nimorazole, nitroxoline, norfloxacin, novobiocin, ofloxacin, oleandomycin, opiniazide, oxacillin, oxophenarsine, oxolinic acid, oxytetracycline, panipenam, paromycin, pazufloxacin, pefloxacin, penicillin G potassium salt, penicillin N, penicillin O, penicillin V, penethamate hydroiodide, pentamidine, phenamidine, phenethicillin potassium salt, phenyl aminosalicyclate, pipacycline, pipemidic acid, piperacillin, pirlimycin, piromidic acid, pivampicillin, pivcefalexin, profiromycin, propamidine, propicillin, protionamide, puraltadone, puromycin, pyrazinamide, pyrimethamine, quinacillin, quinacrine, quinapyramine, quintine, ribostamycin, rifabutine, rifamide, rifampin, rifamycin, rifanpin, rifapentine, rifaxymine, ritipenem, rokitamycin, rolitetracycline, rosamycin, rufloxacin, salazosulfadimidine, salinazid, sancycline, sarafloxacin, sedacamycin, secnidazole, sisomycin, sparfloxacin, spectinomycin, spiramycin, spiramycin I, spiramycin II, spiramycin III, stilbamidine, streptomycin, streptonicizid, sulbactam, sulbenicillin, succisulfone, sulfanilamide, sulfabenzamide, sulfacetamide, sulfachloropyridazine, sulfachrysoidine, sulfacytine, sulfadiazine, sulfadicramide, sulfadimethoxine, sulfadoxine, sulfadrazine, sulfaetidol, sulfafenazol, sulfaguanidine, sulfaguanole, sulfalene, sulfamerazine, sulfameter, sulfamethazine, sulfamethizole, sulfamethomidine, sulfamethoxazole, sulfamethoxypyridazine, sulfamethylthiazol, sulfamethylthiazole, sulfametrole, sulfamidochrysoidine, sulfamoxole, sulfanilamide, 4-sulfanilamido salicylic acid, 4-4′-sulfanilylbenzylamine, p-sulfanilylbenzylamine, 2-p-sulfinylanilinoethanol, sulfanilylurea, sulfoniazide, sulfaperine, sulfaphenazole, sulfaproxyline, sulfapyrazine, sulfapyridine, sulfathiazole, sulfaethidole, sulfathiourea, sulfisomidine, sulfasomizole, sulfasymazine, sulfisoxazole, 4,4′-sulfinyldianiline, N⁴-sulfanilylsulfanilamide, N-sulfanilyl-3,4-xylamide, sultamicillin, talampicillin, tambutol, taurolidine, teiclplanin, temocillin, tetracycline, tetroxoprim, thiabendazole, thiazolsulfone, tibezonium iodide, ticarcillin, tigemonam, tinidazole, tobramycin, tosufloxacin, trimethoprim, troleandromycin, trospectomycin, trovafloxacin, tubercidine, miokamycin, oleandomycin, troleandromycin, vancomycin, verazide, viomycin, virginiamycin, zalcitabine,

In other embodiments the antimicrobial compound is an antiviral compound, including but not limited to, acyclovir, amatadine, cidofovir, cytarabine, didanosine, dideoxyadenosine, edoxudine, famciclovir, floxuridine, gancyclovir, idoxuridine, indanavir, kethoxal, lamivudine, MADU, penciclovir, podophyllotoxin, ribavirine, rimantadine, saquinavir, sorivudine, stavudine, trifluridine, valacyclovir, vidarabine, xenazoic acid, zalcitabine, zidovudine, and the like.

In another embodiment the antimicrobial compound is clvdaptomycin, duramycin, nafcillin, tigecycline, PA-1806, PA-2794, and the like.

Suitable adenosine antagonists, include, but are not limited to, 8-cyclopentyl-1,3-dipropylxanthine (CPX), and the like.

Suitable LTB4 antagonists, include, but are not limited to, amelubant, and the like.

Suitable mucoregulators, include, but are not limited to, talniflumate, MSI-2216, ML-03, INO-4995. and the like.

Suitable purine agonists, include, but are not limited to, P2Y2 agonist, such as, for example, INS-37217, uridine 5′triphosphate, diquafosol tetrasodium, and the like.

The contemplated compounds of the invention are described more fully in the literature, such as in Goodman and Gilman, The Pharmacological Basis of Therapeutics (9th Edition), McGraw-Hill, (1996); Merck Index on CD-ROM, 13^{th} Edition; STN Express, file phar and file registry, the disclosures of each of which are incorporated by reference herein in their entirety.

In one embodiment the compounds of the invention are antimicrobial compounds, such as, amikacin, azetreonam, azithromycin, colistin, duramycin, gentamycin, tigecycline, tobramycin, vancomycin, PA-1806 and PA-2794. In other embodiments, the compounds of the invention are aztrenam, duramycin or tobramycin.

In another embodiment the compounds of the invention are antimicrobial compounds, adenosine antagonists, LTB4 antagonists, mucoregulators and purine agonists, that must contain or be modified to contain one or more of the following functionalities: a carboxylic acid group (—COOH), a hydroxyl group (—OH), a thiol group (—SH) and/or a primary or secondary amine group (—NH). The compounds of the invention are nitrosated and/or nitrosylated through one or more of these functionalities such as oxygen (hydroxyl condensation), sulfur (sulfhydryl condensation) and/or nitrogen.

In one embodiment, the invention describes nitrosated compounds of the invention that are nitrosated antimicrobial compounds, nitrosated adenosine antagonists, nitrosated LTB4 antagonists, nitrosated mucoregulators and nitrosated purine agonists. In one embodiment, the nitrosated antimicrobial compounds are nitrosated daptomycin, nitrosated duramycin, nitrosated nafcillin, nitrosated tigecycline, nitrosated PA-1806, nitrosated PA-2794; the nitrosated adenosine agonist is nitrosated CPX; the nitrosated LTB4 antagonist is nitrosated amelubant; the nitrosated mucoregulators are nitrosated talniflumate, nitrosated MSI-2216, nitrosated ML-03, nitrosated INO-4995; the nitrosated purine agonists are nitrosated P2Y2 agonist, nitrosated INS-37217, nitrosated uridine 5′triphosphate, nitrosated diquafosol tetrasodium. The nitrosated antimicrobial compounds, nitrosated adenosine antagonists, nitrosated LTB4 antagonists, nitrosated mucoregulators and nitrosated purine agonists are nitrosated by containing at least one nitrosated carboxylic acid group (—C(O)K), nitrosated hydroxyl group (—OK), nitrosated thiol group (—SK) and/or primary or secondary nitrosated amine group (—NK);

wherein:

K is —W′ₐ-E_{b}-(C(Rₑ)(R_{f}))_{p′}-E_{c}-(C(Rₑ)(R_{f}))ₓ—W′_{d}—(C(Rₑ)(R_{f}))_{y}—W′ᵢ-Eⱼ-W′_{g}-(C(Rₑ)(R_{f}))_{z}—U—NO₂;

a, b, c, d, g, i and j are each independently an integer from 0 to 3;

p′, x, y and z are each independently an integer from 0 to 10;

W′ at each occurrence is independently —C(O)—, —C(S)—, -T′-, —(C(Rₑ)(R_{f}))ₕ—, an alkyl group, an aryl group, a heterocyclic ring, an arylheterocyclic ring, or —(CH₂CH₂O)_{q′}—;

E at each occurrence is independently -T′-, an alkyl group, an aryl group, —(C(Rₑ)(R_{f})ₕ—, a heterocyclic ring, an arylheterocyclic ring, or —(CH₂CH₂O)_{q′}—;

T′ at each occurrence is independently a covalent bond, a carbonyl, an oxygen, —S(O)ₒ— or —N(Rₐ)Rᵢ;

h is an integer form 1 to 10;

q′ is an integer from 1 to 5;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, W′ₕ, —(CH₂)—U—V₁, or —(C(R_{g})(Rₕ))ₖ—U—V₂, or Rₑ and R_{f} taken together with the carbons to which they are attached form a carbonyl, a methanthial, a heterocyclic ring, a cycloalkyl group, an aryl group, an oxime, a hydrazone or a bridged cycloalkyl group;

R_{g} and Rₕ at each occurrence are independently Rₑ;

k is an integer from 1 to 3;

U at each occurrence is independently a covalent bond, a carbonyl, an oxygen, —S(O)ₒ— or —N(Rₐ)Rᵢ;

V₁ is —NO or —NO₂;

o is an integer from 0 to 2;

Rₐ is a lone pair of electrons, a hydrogen or an alkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U—V₁)(Rₑ)(R_{f}), a bond to an adjacent atom creating a double bond to that atom, —(N₂O₂—)⁻.M⁺, wherein M⁺ is an organic or inorganic cation; and

with the proviso that the nitrosated compounds of the invention must contain at least one NO₂ group; wherein the at least one NO₂ group is linked to the compound through an oxygen atom, a nitrogen atom or a sulfur atom.

In cases where multiple designations of variables which reside in sequence are chosen as a “covalent bond” or the integer chosen is 0, the intent is to denote a single covalent bond connecting one radical to another. For example, E₀ would denote a covalent bond, while E₂ denotes (E-E) and (C(R₄)(R₄))₂ denotes —C(R₄)(R₄)—C(R₄)(R₄)—.

In another embodiment, the invention describes nitrosated antimicrobial compounds, nitrosated adenosine antagonists, nitrosated LTB4 antagonists, nitrosated mucoregulators and nitrosated purine agonists, wherein the antimicrobial compounds are acediasulfone, aceturate, acetyl sulfametossipirazine, acetyl sulfamethoxypyrazine, acranil, albendazole, alexidine, amatadine, ambazone, amdinocillin, amikacin, p-aminosalicylic acid, p-aminosalicylic acid hydrazine, amoxicillin, ampicillin, anisomycin, apalcillin, apicyclin, apramycin, arbekacin, argininsa, aspoxicillin, azidamfenicol, azidocillin, azithromycin, azlocillin, aztreonam, bacampicillin, benzoylpas, benzyl penicillin acid, benzyl sulfamide, bicozamycin, bipenam, brodimoprim, capreomycin, carbenicillin, carbomycin, cafazedone, carindacillin, carumonam, cefcapene pivoxil, cefaclor, cefadroxil, cefafroxil, cefamandole, cefatamet, cefatrizine, cefazedone, cefazolin, cefbuperazone, cefclidin, cefdinir, cefditoren, cefixime, cefmenoxime, cefmetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftezole, ceftibuten, ceftiofur, ceftizoxime, ceftriaxone, cefuroxime, cefuzonam, cephacetrile sodium, cephadrine, cephalexin, cephaloglycin, cephaloridine, cephalosporin C, cephalothin, cephapirin sodium, cephradine, chloramphenicol, chlorotetracycline, cinoxacin, ciprofloxacin, claritromycin, clavulanic acid, clinafloxacin, clindamycin, clofazimine, clofoctal, clometocillin, clomocycline, cloxacillin, cloxyquin, cyclacilline, cycloserine, danoflaxcin, dapsone, deoxycycline, deoxydihydrostreptomycin, dibekacin, dicloxacillin, difloxacin, dihydrostreptomycin, dimetridazole, diminazene, dirirtomycin, eflornithine, enrofloxacin, enviomycin, epicillin, erythromycin, etacillin, ethambutol, ethionamide, famcyclovir, fenbecillin, fleroxacin, flomoxef, floxacillin, flumequine, furonazide, fortimycin, furazolium chloride, gentamycin, glyconiazide, grepafloxacin, guamecycline, halofuginone, hetacillin, homidium, hydroxyl-stilbamidine, ibostamycin, imidocarb, imipenam, ipronidazole, isoniazide, josamycin, inosine, lauroguadine, lenampicillin, lincomycin, lomefloxacin, loracarbef, lymecyclin, mafenide, mebendazole, meclocyclin, meropenem, metampicillin, metacicline, methacycline, methicillin sodium, metronidazole, 4′-(methylsulfamoyl) sulfanilanilide, mezlocillin, meziocillin, micronomycin, midecamycin A₁, minocycline, miocamycin, miokamycin, morfazinamide, moxalactam, mupirocin, myxin, nadifloxacin, nalidixic acid, negamycin, neomycin, netlimycin, nifurfoline, nifurpirinol, nifurprazine, nimorazole, nitroxoline, norfloxacin, novobiocin, ofloxacin, oleandomycin, opiniazide, oxacillin, oxophenarsine, oxolinic acid, oxytetracycline, panipenam, paromycin, pazufloxacin, pefloxacin, penicillin G potassium salt, penicillin N, penicillin O, penicillin V, penethamate hydroiodide, pentamidine, phenamidine, phenethicillin potassium salt, phenyl aminosalicyclate, pipacycline, pipemidic acid, piperacillin, pirlimycin, piromidic acid, pivampicillin, pivcefalexin, profiromycin, propamidine, propicillin, protionamide, puraltadone, puromycin, pyrazinamide, pyrimethamine, quinacillin, quinacrine, quinapyramine, quintine, ribostamycin, rifabutine, rifamide, rifampin, rifamycin, rifanpin, rifapentine, rifaxymine, ritipenem, rokitamycin, rolitetracycline, rosamycin, rufloxacin, salazosulfadimidine, salinazid, sancycline, sarafloxacin, sedacamycin, secnidazole, sisomycin, sparfloxacin, spectinomycin, spiramycin, spiramycin I, spiramycin II, spiramycin III, stilbamidine, streptomycin, streptonicizid, sulbactam, sulbenicillin, succisulfone, sulfanilamide, sulfabenzamide, sulfacetamide, sulfachloropyridazine, sulfachrysoidine, sulfacytine, sulfadiazine, sulfadicramide, sulfadimethoxine, sulfadoxine, sulfadrazine, sulfaetidol, sulfafenazol, sulfaguanidine, sulfaguanole, sulfalene, sulfamerazine, sulfameter, sulfamethazine, sulfamethizole, sulfamethomidine, sulfamethoxazole, sulfamethoxypyridazine, sulfamethylthiazol, sulfamethylthiazole, sulfametrole, sulfamidochrysoidine, sulfamoxole, sulfanilamide, 4-sulfanilamido salicylic acid, 4-4′-sulfanilylbenzylamine, p-sulfanilylbenzylamine, 2-p-sulfinylanilinoethanol, sulfanilylurea, sulfoniazide, sulfaperine, sulfaphenazole, sulfaproxyline, sulfapyrazine, sulfapyridine, sulfathiazole, sulfaethidole, sulfathiourea, sulfisomidine, sulfasomizole, sulfasymazine, sulfisoxazole, 4,4′-sulfinyldianiline, N⁴-sulfanilylsulfanilamide, N-sulfanilyl-3,4-xylamide, sultamicillin, talampicillin, tambutol, taurolidine, teiclplanin, temocillin, tetracycline, tetroxoprim, thiabendazole, thiazolsulfone, tibezonium iodide, ticarcillin, tigemonam, tinidazole, tobramycin, tosufloxacin, trimethoprim, troleandromycin, trospectomycin, trovafloxacin, tubercidine, miokamycin, oleandomycin, troleandromycin, vancomycin, verazide, viomycin, virginiamycin, zalcitabine, acyclovir, amatadine, cidofovir, cytarabine, didanosine, dideoxyadenosine, edoxudine, famciclovir, floxuridine, gancyclovir, idoxuridine, indanavir, kethoxal, lamivudine, MADU, penciclovir, podophyllotoxin, ribavirine, rimantadine, saquinavir, sorivudine, stavudine, trifluridine, valacyclovir, vidarabine, xenazoic acid, zalcitabine, zidovudine, daptomycin, duramycin, nafcillin, tigecycline, PA-1806, PA-2794; the adenosine agonist is CPX; the LTB4 antagonist is amelubant, the mucoregulators are talniflumate, MSI-2216, ML-03, INO-4995; the purine agonists are P2Y2 agonist, INS-37217, uridine 5′triphosphate, diquafosol tetrasodium; wherein the nitrosated antimicrobial compounds, nitrosated adenosine antagonists, nitrosated LTB4 antagonists, nitrosated mucoregulators and nitrosated purine agonists are nitrosated by containing or modified to contain at least one nitrosated carboxylic acid group (—C(O)X), nitrosated hydroxyl group (—OX), nitrosated thiol group (—SX) and/or primary or secondary nitrosated amine group (—NX);

wherein X is:

(1) —Y—(CR₄R₄′)ₚ-T-(CR₄R₄′)ₚ—ONO₂;

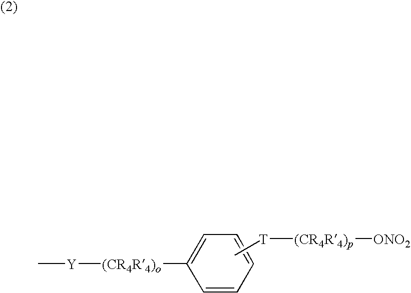

wherein T is ortho, meta or para;

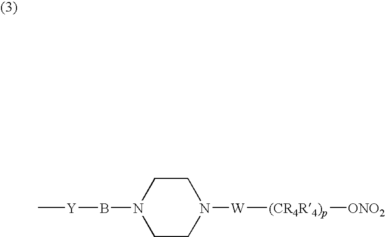

(4) —Y—(CR₄C₄′)ₚ—V—B-T-(CR₄R₄′)ₚ—ONO₂;

(5) —Y—(CR₄R₄′)ₚ-T-C(O)—(CR₄R₄′)ₒ—(CH₂)—ONO₂;

(6) —Y—(CR₄R₄′)ₚ—C(Z)-(CH₂)_{q}-T-(CR₄R₄′)_{q}—(CH₂)—ONO₂;

(7) —Y—(CR₄R₄′)ₚ-T-(CH₂)_{q}—V—(CR₄R₄′)_{q}—(CH₂)—ONO₂;

(8) —Y—(CR₄R₄′)ₚ—V—(CH₂)_{q}—V—(CR₄R₄′)_{q}—(CH₂)—ONO₂;

(9) —Y—(CR₄R₄′)ₒ—(W)_{q}—(CR₄R₄′)ₒ—(CH₂)—ONO₂;

(10) —NRⱼ—O—(CH₂)ₒ—V—(C₄R₄′)_{q}—(CH₂)—ONO₂;

(11) —NRⱼ—O—(CH₂)ₒ—(W)_{q}—(CR₄R₄′)_{q}—CH₂)—ONO₂;

(12) —O—NRⱼ—(CH₂)ₒ—(W)_{q}—(CR₄R₄′)_{q}—CH₂)—ONO₂;

(13) —Y—(CH₂)ₒ—(W)_{q}(CH₂)ₒ—V—(CR₄R₄′)ₒ-Q′-(CR₄R₄′)ₒ—(CH₂)—ONO₂;

(14) —Y—(CR₄R₄′)ₚ—V—(CH₂)ₒ—(W)_{q}—(CR₄R₄′)_{q}—(CH₂)—ONO₂;

(15) —O—NRⱼ—(CH₂)ₒ—V—(CR₄R₄′)_{q}—(CH₂)—ONO₂;

(16) —Y—(CR₄R₄′)ₒ-Q′-(CR₄R₄′)ₒ—V—(CR₄R₄′)ₒ—(CH₂)—ONO₂;

(17) —Y—(CR₄R₄′)ₒ-Q′-(CR₄R₄′)ₒ—(W)_{q}—(CR₄R₄′)ₒ—(CH₂)ₒ—ONO₂;

(18) —Y—(CR₄R₄′)ₚ-T-(CR₄R₄′)ₚ-Q′-(CR₄R₄′)ₒ—(CH₂)—ONO₂;

(19) —Y—(CR₄R₄′)_{q}—C(Z)-(CR₄R₄′)ₒ—(CH₂)—ONO₂;

(20) —Y—(CR₄R₄′)ₚ-Q′-(CR₄R₄′)ₒ—(CH₂)—ONO₂;

(21) —Y—(CR₄R₄′)_{q}—P(O)MM′;

(22) —Y—(CR₄R₄′)ₒ-Q′-(CR₄R₄′)ₒ—(CH₂)—ONO₂;

(23) —Y—(CR₄R₄′)ₒ-Q′-(CR₄R₄′)ₒ-T-(CR₄R₄′)ₒ—(CH₂)—ONO₂;

(24) —Y—(CR₄R₄′)_{q}—(W)_{q}—(CR₄R₄′)ₒ-Q′-(CR₄R₄′)ₒ—(CH₂)—ONO₂;

(25) —Y—(CR₄R₄′)_{q}—V—(CR₄R₄′)ₒ-Q′-(CR₄R₄′)ₒ—(CH₂)—ONO₂;

(26) —Y—(CR₄R₄′)ₚ-(T)ₒ-(W)_{q}—(CR₄R₄′)ₒ—(CH₂)—ONO₂;

(27) —Y—(CR₄R₄′)ₚ—(W)_{q}-(T)ₒ-(CR₄R₄′)ₒ—(CH₂)—ONO₂;

(28) —Y—(CR₄R₄′)_{q}—C(Z)-V—(CR₄R₄′)_{q}—(CH₂)—ONO₂;

(29) —Y—(CR₄R₄′)ₒ—C(R₄)(ONO₂)—(CR₄R₄′)_{q}(T)ₒ-(W)_{q}-(T)ₒ-(CR₄R₄′)ₒ—R₅;

(30) —Y—(CR₄R₄′)ₒ—V—(CR₄R₄′)ₒ-Q′-(CR₄R₄′)ₒ—(CH₂)—ONO₂;

(31) —Y—(CR₄R₄′)_{q}—C(Z)-Q′-(CR₄R₄′)ₒ—(CH₂)—ONO₂;

(32) —Y—(CR₄R₄′)ₚ—V—(CR₄R₄′)ₚ—(CH₂)—ONO₂;

(33) —Y—(CR₄R₄′)ₚ—V—(CH₂)_{q}-(T)ₒ-(CR₄R₄′)_{q}—(CH₂)—ONO₂;

(34) —Y—(CR₄R₄′)ₚ-(T)ₒ-Q′-(T)ₒ-(CR₄R₄′)_{q}—(CH₂)—ONO₂;

(35) —Y—(CR₄R₄′)_{q}—C(Z)-(CR₄R₄′)_{q}—V—(C R₄R₄′)ₒ-Q′-(CR₄R₄′)ₒ—ONO₂;

(36) —Y—(CR₄R₄′)_{q}—C(Z)-(CR₄R₄′)_{q}(W)_{q}—(CR₄R₄′)ₒ-Q′-(CR₄R₄′)ₒ—(CH₂)—ONO₂;

(37) —NRⱼ—O—(CH₂)ₒ—V—(CR₄R₄′)ₒ-Q′-(CH₂)—ONO₂;

(38) —NRⱼ—O—(CH₂)ₒ—(W)_{q}—(CR₄R₄′)ₒ-Q′-(CH₂)—ONO₂;

(39) —O—NRⱼ—(CH₂)ₒ—(W)_{q}—(CR₄R₄′)ₒ-Q′-(CH₂)—ONO₂;

(40) —O—NRⱼ—(CH₂)ₒ—V—(CR₄R₄′)ₒ-Q′-(CH₂)—ONO₂;

(41) —NRⱼ—NRⱼ—(CR₄R₄′)ₚ—(W)_{q}-(T)ₒ-(CR₄R₄′)ₒ—(CH₂)—ONO₂; or

(42) —Y—(CR₄R₄′)ₒ-Q′-(CR₄R₄′)ₒ—ONO₂; or

(43) —Y—(CR₄R₄′)ₒ—V—(CR₄R₄′)ₒ-Q-(CR₄R⁴′)ₒ—ONO₂;

R₄ and R₄′ at each occurrence are independently a hydrogen, lower alkyl group, —OH, —CH₂OH, —ONO₂, —NO₂ or —CH₂ONO₂; or R₄ and R₄′ taken together with the carbon atom to which they are attached are a cycloalkyl group or a heterocyclic ring;

V is —C(O)-T-, -T-C(O)—, -T-C(O)-T or T-C(O)—C(O)-T;

W is a covalent bond or a carbonyl group;

T at each occurrence is independently an oxygen, (S(O)ₒ)ₒ or NRⱼ;

Rⱼ is a hydrogen, an alkyl group, an aryl group, a heterocyclic ring, an alkylcarbonyl group, an alkylaryl group, an alkylsulfinyl group, an alkylsulfonyl group, an arylsulfinyl group, an arylsulfonyl group, a sulfonamido group, a N-alkylsulfonamido group, a N,N-diarylsulfonamido group, a N-arylsulfonamido group, a N-alkyl-N-arylsulfonamido group, a carboxamido group or a hydroxyl group;

p at each occurrence is independently an integer from 1 to 6;

q at each occurrence is independently an integer from 1 to 3;

o at each occurrence is independently an integer from 0 to 2;

Y is independently a covalent bond, a carbonyl, an oxygen, —S(O)ₒ— or —NRⱼ;

B is either phenyl or (CH₂)ₒ;

Q′ is a cycloalkyl group, a heterocyclic ring or an aryl group;

Z is (═O), (═N—OR₅), (═N—NR₅R′₅) or (═CR₅R′₅);

M and M′ are each independently —O⁻H₃N⁺—(CR₄R′₄)_{q}—CH₂ONO₂ or -T-(CR₄R′₄)ₒ—CH₂ONO₂; and

R₅ and R₅′ at each occurrence are independently a hydrogen, a hydroxyl group, an alkyl group, an aryl group, an alkylsulfonyl group, an arylsulfonyl group, a carboxylic ester, an alkylcarbonyl group, an arylcarbonyl group, a carboxamido group, an alkoxyalkyl group, an alkoxyaryl group, a cycloalkyl group or a heterocyclic ring; and

with the proviso that the nitrosated compounds of the invention must contain at least one NO₂ group; wherein the at least one NO₂ group is linked to the compound through an oxygen atom, a nitrogen atom or a sulfur atom.

It is also to be understood that the invention is intended to include within its scope compounds which may exist in more than one resonance form and the effects that the resonance form may have on the positions at the X substituent designated in the compounds described herein.

In preferred embodiments of the invention for the nitrosated antimicrobial compounds, nitrosated adenosine antagonists, nitrosated LTB4 antagonists, nitrosated mucoregulators and nitrosated purine agonists, and pharmaceutically acceptable salts thereof, X is:

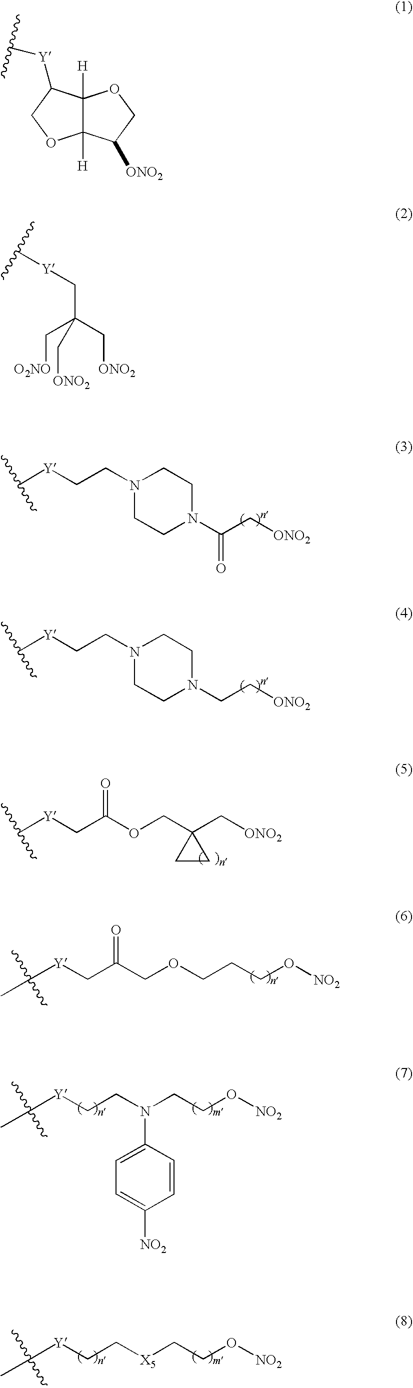

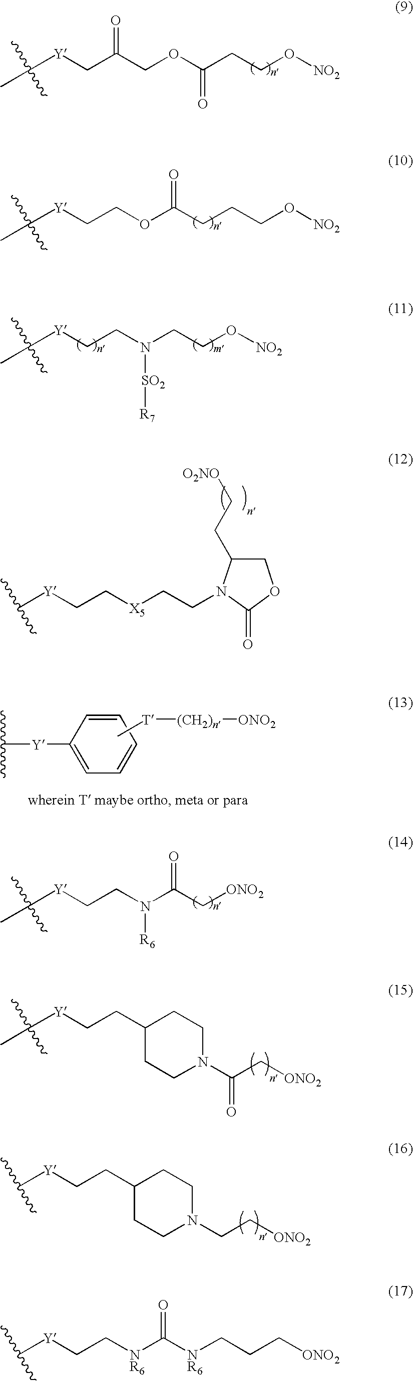

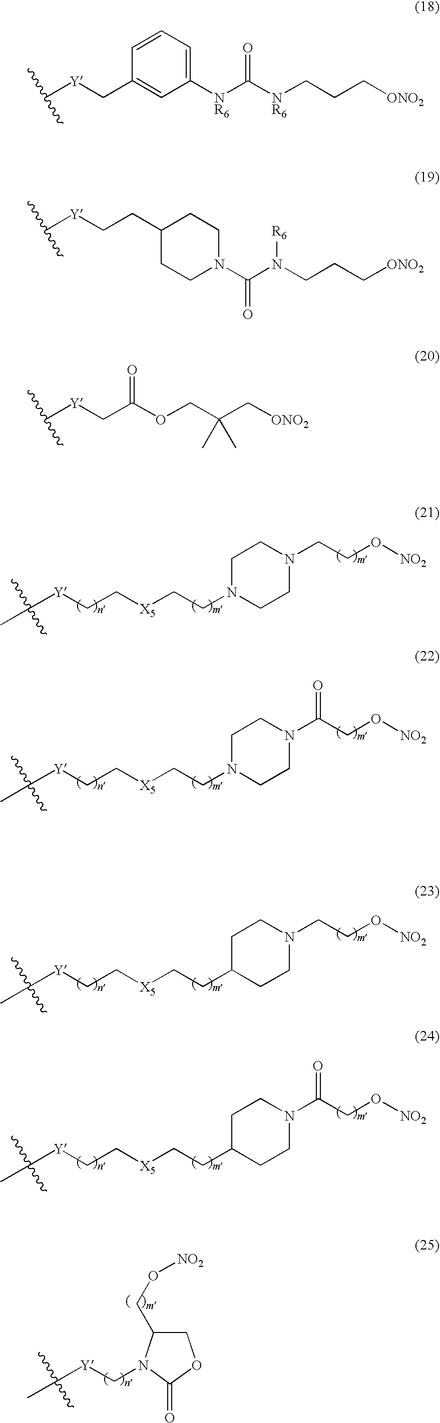

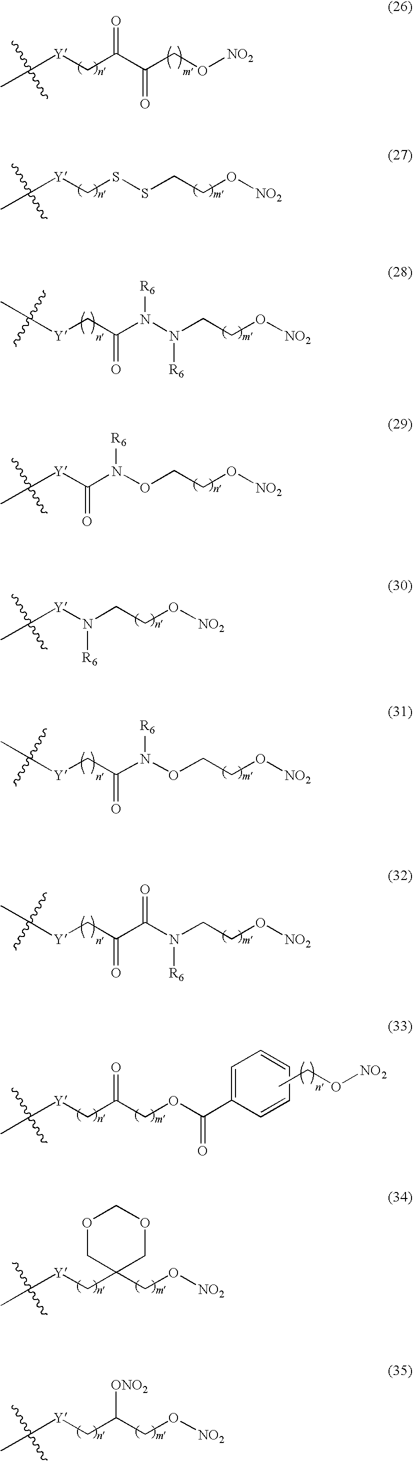

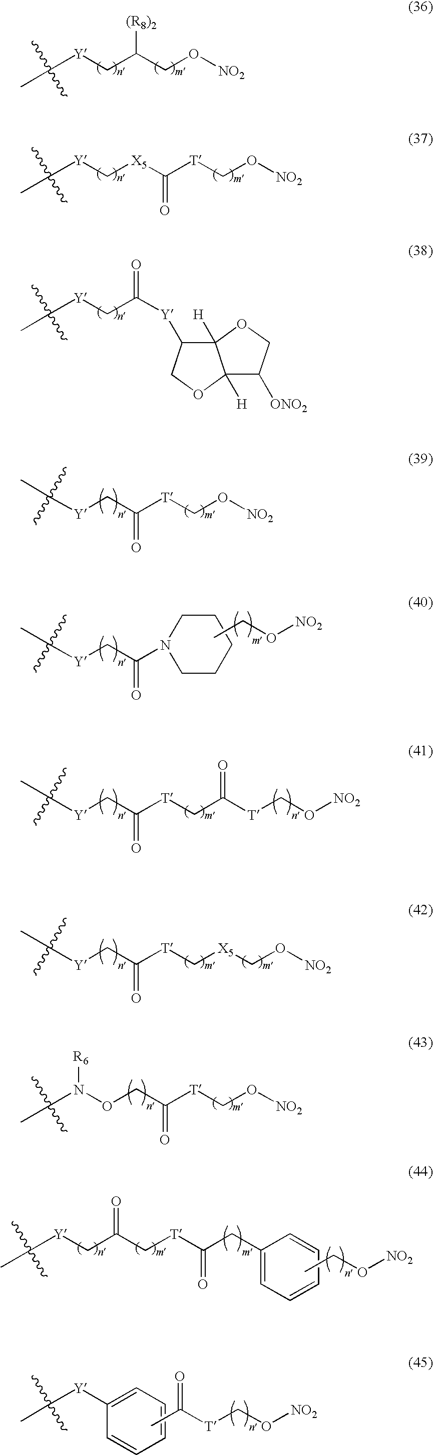

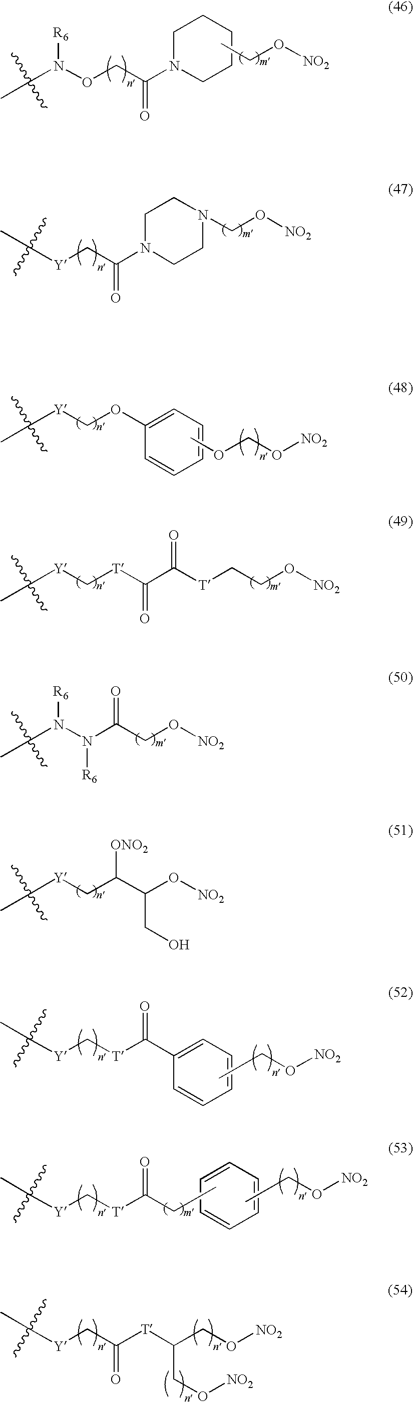

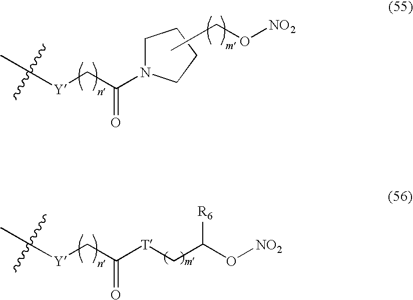

wherein:

Y′ is oxygen or sulfur;

T′ is oxygen, sulfur or NR₆;

X₅ is oxygen, (S(O)ₒ)ₒ or NR₆;

R₆ is a hydrogen, a lower alkyl group, an aryl group;

R₇ is a lower alkyl group or an aryl group;

R₈ at each occurrence is independently is a hydrogen, a hydroxyl group, a lower alkyl group, an aryl group, —NO₂, —CH₂—ONO₂ or —CH₂—OH;

n′ and m′ are each independently an integer from 0 to 10; and

o is as an integer from 0 to 2.

In another embodiment of the invention, the nitrosated compounds of the invention do not include the compounds disclosed in WO 02/51385, WO 01/54691, WO 00/61549, WO 00/61541, WO 00/61537, the disclosures of each of which are incorporated by reference herein in their entirety.

In one embodiment the nitrosated compounds of the invention are preferably nitrosated antimicrobial compounds, preferably, nitrosated amikacin, nitrosated azetreonam, nitrosated azithromycin, nitrosated colistin, nitrosated duramycin, nitrosated gentamycin, nitrosated tigecycline, nitrosated tobramycin, nitrosated vancomycin, nitrosated PA-1806 and/or nitrosated PA-2794, and more preferably nitrosated aztrenam, nitrosated duramycin and/or nitrosated tobramycin.

In another embodiment, the invention describes nitrosated antimicrobial compounds of the invention and pharmaceutically acceptable salts thereof. In one embodiment, the nitrosated antimicrobial pharmaceutically acceptable salts do not include the nitrate salt.

In another embodiment, the invention describes nitrosylated antimicrobial compounds, nitrosylated adenosine antagonists, nitrosylated LTB4 antagonists, nitrosylated mucoregulators and nitrosylated purine agonists, wherein the antimicrobial compounds are acediasulfone, aceturate, acetyl sulfametossipirazine, acetyl sulfamethoxypyrazine, acranil, albendazole, alexidine, amatadine, ambazone, amdinocillin, amikacin, p-aminosalicylic acid, p-aminosalicylic acid hydrazine, amoxicillin, ampicillin, anisomycin, apalcillin, apicyclin, apramycin, arbekacin, argininsa, aspoxicillin, azidamfenicol, azidocillin, azithromycin, azlocillin, aztreonam, bacampicillin, benzoylpas, benzyl penicillin acid, benzyl sulfamide, bicozamycin, bipenam, brodimoprim, capreomycin, carbenicillin, carbomycin, cafazedone, carindacillin, carumonam, cefcapene pivoxil, cefaclor, cefadroxil, cefafroxil, cefamandole, cefatamet, cefatrizine, cefazedone, cefazolin, cefbuperazone, cefclidin, cefdinir, cefditoren, cefixime, cefmenoxime, cefmetazole, cefminox, cefodizime, cefonicid, cefoperazone, ceforanide, cefotaxime, cefotetan, cefotiam, cefoxitin, cefozopran, cefpimizole, cefpiramide, cefpirome, cefpodoxime proxetil, cefprozil, cefroxadine, cefsulodin, ceftazidime, cefteram, ceftezole, ceftibuten, ceftiofur, ceftizoxime, ceftriaxone, cefuroxime, cefuzonam, cephacetrile sodium, cephadrine, cephalexin, cephaloglycin, cephaloridine, cephalosporin C, cephalothin, cephapirin sodium, cephradine, chloramphenicol, chlorotetracycline, cinoxacin, ciprofloxacin, claritromycin, clavulanic acid, clinafloxacin, clindamycin, clofazimine, clofoctal, clometocillin, clomocycline, cloxacillin, cloxyquin, cyclacilline, cycloserine, danoflaxcin, dapsone, deoxycycline, deoxydihydrostreptomycin, dibekacin, dicloxacillin, difloxacin, dihydrostreptomycin, dimetridazole, diminazene, dirirtomycin, eflornithine, enrofloxacin, enviomycin, epicillin, erythromycin, etacillin, ethambutol, ethionamide, famcyclovir, fenbecillin, fleroxacin, flomoxef, floxacillin, flumequine, furonazide, fortimycin, furazolium chloride, gentamycin, glyconiazide, grepafloxacin, guamecycline, halofuginone, hetacillin, homidium, hydroxyl-stilbamidine, ibostamycin, imidocarb, imipenam, ipronidazole, isoniazide, josamycin, inosine, lauroguadine, lenampicillin, lincomycin, lomefloxacin, loracarbef, lymecyclin, mafenide, mebendazole, meclocyclin, meropenem, metampicillin, metacicline, methacycline, methicillin sodium, metronidazole, 4′-(methylsulfamoyl)sulfanilanilide, mezlocillin, meziocillin, micronomycin, midecamycin A₁, minocycline, miocamycin, miokamycin, morfazinamide, moxalactam, mupirocin, myxin, nadifloxacin, nalidixic acid, negamycin, neomycin, netlimycin, nifurfoline, nifurpirinol, nifurprazine, nimorazole, nitroxoline, norfloxacin, novobiocin, ofloxacin, oleandomycin, opiniazide, oxacillin, oxophenarsine, oxolinic acid, oxytetracycline, panipenam, paromycin, pazufloxacin, pefloxacin, penicillin G potassium salt, penicillin N, penicillin O, penicillin V, penethamate hydroiodide, pentamidine, phenamidine, phenethicillin potassium salt, phenyl aminosalicyclate, pipacycline, pipemidic acid, piperacillin, pirlimycin, piromidic acid, pivampicillin, pivcefalexin, profiromycin, propamidine, propicillin, protionamide, puraltadone, puromycin, pyrazinamide, pyrimethamine, quinacillin, quinacrine, quinapyramine, quintine, ribostamycin, rifabutine, rifamide, rifampin, rifamycin, rifanpin, rifapentine, rifaxymine, ritipenem, rokitamycin, rolitetracycline, rosamycin, rufloxacin, salazosulfadimidine, salinazid, sancycline, sarafloxacin, sedacamycin, secnidazole, sisomycin, sparfloxacin, spectinomycin, spiramycin, spiramycin L, spiramycin II, spiramycin III, stilbamidine, streptomycin, streptonicizid, sulbactam, sulbenicillin, succisulfone, sulfanilamide, sulfabenzamide, sulfacetamide, sulfachloropyridazine, sulfachrysoidine, sulfacytine, sulfadiazine, sulfadicramide, sulfadimethoxine, sulfadoxine, sulfadrazine, sulfaetidol, sulfafenazol, sulfaguanidine, sulfaguanole, sulfalene, sulfamerazine, sulfameter, sulfamethazine, sulfamethizole, sulfamethomidine, sulfamethoxazole, sulfamethoxypyridazine, sulfamethylthiazol, sulfamethylthiazole, sulfametrole, sulfamidochrysoidine, sulfamoxole, sulfanilamide, 4-sulfanilamido salicylic acid, 4-4′-sulfanilylbenzylamine, p-sulfanilylbenzylamine, 2-p-sulfinylanilinoethanol, sulfanilylurea, sulfoniazide, sulfaperine, sulfaphenazole, sulfaproxyline, sulfapyrazine, sulfapyridine, sulfathiazole, sulfaethidole, sulfathiourea, sulfisomidine, sulfasomizole, sulfasymazine, sulfisoxazole, 4,4′-sulfinyldianiline, N⁴-sulfanilylsulfanilamide, N-sulfanilyl-3,4-xylamide, sultamicillin, talampicillin, tambutol, taurolidine, teiclplanin, temocillin, tetracycline, tetroxoprim, thiabendazole, thiazolsulfone, tibezonium iodide, ticarcillin, tigemonam, tinidazole, tobramycin, tosufloxacin, trimethoprim, troleandromycin, trospectomycin, trovafloxacin, tubercidine, miokamycin, oleandomycin, troleandromycin, vancomycin, verazide, viomycin, virginiamycin, zalcitabine, acyclovir, amatadine, cidofovir, cytarabine, didanosine, dideoxyadenosine, edoxudine, famciclovir, floxuridine, gancyclovir, idoxuridine, indanavir, kethoxal, lamivudine, MADU, penciclovir, podophyllotoxin, ribavirine, rimantadine, saquinavir, sorivudine, stavudine, trifluridine, valacyclovir, vidarabine, xenazoic acid, zalcitabine, zidovudine, daptomycin, duramycin, nafcillin, tigecycline, PA-1806, PA-2794; the adenosine agonist is CPX; the LTB4 antagonist is amelubant; the mucoregulators, are talniflumate, MSI-2216, ML-03, INO-4995; the purine agonists are P2Y2 agonist, INS-37217, uridine 5′triphosphate, diquafosol tetrasodium; wherein the nitrosylated antimicrobial compounds, nitrosylated adenosine antagonists, nitrosylated LTB4 antagonists, nitrosylated mucoregulators and nitrosylated purine agonists are nitrosylated by containing or are modified to contain at least one nitrosylated carboxylic acid group (—C(O)K₁), nitrosylated hydroxyl group (—O K₁), nitrosylated thiol group (—S K₁) and/or primary or secondary nitrosylated amine group (—N K₁);

K₁ is —W′ₐ-E_{b}-(C(Rₑ)(R_{f}))_{p′}-E_{c}-(C(Rₑ)(R_{f}))_{y}—W′ᵢ-Eⱼ-W′_{g}—(C(Rₑ)(R_{f}))_{z}—U—NO;

wherein:

a, b, c, d, g, i, j, p′, x, y, z, W′, E, Rₑ, R_{f} and U are as defined herein; and with the proviso that the nitrosylated compounds of the invention must contain at least one NO group; wherein the at least one NO group is linked to the compound through an oxygen atom, a nitrogen atom or a sulfur atom.

Compounds of the invention that have one or more asymmetric carbon atoms may exist as the optically pure enantiomers, pure diastereomers, mixtures of enantiomers, mixtures of diastereomers, racemic mixtures of enantiomers, diastereomeric racemates or mixtures of diastereomeric racemates. It is to be understood that the invention anticipates and includes within its scope all such isomers and mixtures thereof.

Another embodiment of the invention describes the metabolites of the nitrosated and/or nitrosylated cardiovascular compounds and pharmaceutically acceptable salts thereof. These metabolites, include but are not limited to, the non-nitrosated and/or nitrosylated derivatives, degradation products, hydrolysis products, and the like, of the nitrosated and/or nitrosylated cardiovascular compounds and pharmaceutically acceptable salts thereof.

Another embodiment of the invention provides processes for making the novel compounds of the invention and to the intermediates useful in such processes. The reactions are performed in solvents appropriate to the reagents and materials used are suitable for the transformations being effected. It is understood by one skilled in the art of organic synthesis that the functionality present in the molecule must be consistent with the chemical transformation proposed. This will, on occasion, necessitate judgment by the routineer as to the order of synthetic steps, protecting groups required, and deprotection conditions. Substituents on the starting materials may be incompatible with some of the reaction conditions required in some of the methods described, but alternative methods and substituents compatible with the reaction conditions will be readily apparent to one skilled in the art. The use of sulfur and oxygen protecting groups is well known for protecting thiol and alcohol groups against undesirable reactions during a synthetic procedure and many such protecting groups are known and described by, for example, Greene and Wuts, Protective Groups in Organic Synthesis, Third Edition, John Wiley & Sons, New York (1999).

The chemical reactions described herein are generally disclosed in terms of their broadest application to the preparation of the compounds of this invention. Occasionally, the reactions may not be applicable as described to each compound included within the disclosed scope. The compounds for which this occurs will be readily recognized by one skilled in the art. In all such cases, either the reactions can be successfully performed by conventional modifications known to one skilled in the art, e.g., by appropriate protection of interfering groups, by changing to alternative conventional reagents, by routine modification of reaction conditions, and the like, or other reactions disclosed herein or otherwise conventional, will be applicable to the preparation of the corresponding compounds of this invention. In all preparative methods, all starting materials are known or readily prepared from known starting materials.

The compounds of the invention are nitrosated and/or nitrosylated through one or more sites such as oxygen, sulfur and/or nitrogen using conventional methods known to one skilled in the art. For example, known methods for nitrosating and/or nitrosylating compounds are described in U.S. Pat. Nos. 5,380,758, 5,859,053, 5,703,073 and 6,297,260; and in WO 94/03421, WO 94/04484, WO 94/12463, WO 95/09831, WO 95/19952, WO 95/30641, WO 97/27749, WO 98/19672, WO 98/21193, WO 00/51988, WO 00/61537, WO 00/61541, WO 00/61604, WO 00/72838, WO 01/00563, WO 01/04082, WO 01/10814, WO 01/12584, WO 01/45703, WO 02/11707, WO 02/30866, WO 02/051385, and in Oae et al, Org. Prep. Proc. Int., 15(3):165-198 (1983), the disclosures of each of which are incorporated by reference herein in their entirety. The methods of nitrosating and/or nitrosylating the compounds described in these references can be applied by one skilled in the art to produce any of the nitrosated and/or nitrosylated compounds described herein. The nitrosated and/or nitrosylated compounds of the invention donate, transfer or release a biologically active form of nitrogen monoxide (i.e., nitric oxide).

Compounds contemplated for use in the invention, e.g., antimicrobial compounds, adenosine antagonists, LTB4 antagonists, mucoregulators and purine agonists that are nitrosated and/or nitrosylated, through one or more sites such as oxygen (hydroxyl condensation), sulfur (sulfhydryl condensation) and/or nitrogen, are, optionally, used in combination with nitric oxide and compounds that release nitric oxide or otherwise directly or indirectly deliver or transfer a biologically active form of nitrogen monoxide to a site of its intended activity, such as on a cell membrane in vivo.

Nitrogen monoxide can exist in three forms: NO⁻ (nitroxyl), NO (nitric oxide) and NO⁺ (nitrosonium); NO is a highly reactive short-lived species that is potentially toxic to cells. This is critical because the pharmacological efficacy of NO depends upon the form in which it is delivered. In contrast to the nitric oxide radical (NO), nitrosonium (NO⁺) does not react with O₂ or O₂— species, and functionalities capable of transferring and/or releasing NO⁺ and NO⁻ are also resistant to decomposition in the presence of many redox metals. Consequently, administration of charged NO equivalents (positive and/or negative) does not result in the generation of toxic by-products or the elimination of the active NO moiety.

The term “nitric oxide” encompasses uncharged nitric oxide (NO) and charged nitrogen monoxide species, preferably charged nitrogen monoxide species, such as nitrosonium ion (NO⁺) and nitroxyl ion (NO⁻). The reactive form of nitric oxide can be provided by gaseous nitric oxide. The nitrogen monoxide releasing, delivering or transferring compounds have the structure F—NO, wherein F is a nitrogen monoxide releasing, delivering or transferring moiety, and include any and all such compounds which provide nitrogen monoxide to its intended site of action in a form active for its intended purpose. The term “NO adducts” encompasses any nitrogen monoxide releasing, delivering or transferring compounds, including, for example, S-nitrosothiols, nitrites, nitrates, S-nitrothiols, sydnonimines, 2-hydroxy-2-nitrosohydrazines, (NONOates), (E)-alkyl-2-((E)-hydroxyimino)-5-nitro-3-hexeneamide (FK-409), (E)-allyl-2-((E)-hydroxyimino)-5-nitro-3-hexeneamines, N-((2Z, 3E)-4-ethyl-2-(hydroxyimino)-6-methyl-5-nitro-3-heptenyl)-3-pyridinecarboxamide (FR 146801), N-nitrosoamines, N-hydroxyl nitrosamines, nitrosimines, diazetine dioxides, oxatriazole 5-imines, oximes, hydroxylamines, N-hydroxyguanidines, hydroxyureas, benzofuroxanes, furoxans as well as substrates for the endogenous enzymes which synthesize nitric oxide.

Suitable NONOates include, but are not limited to, (Z)-1-(N-methyl-N-(6-(N-methyl-ammoniohexyl)amino))diazen-1-ium-1,2-diolate (“MAHMA/NO”), (Z)-1-(N-(3-ammoniopropyl)-N-(n-propyl)amino)diazen-1-ium-1,2-diolate (“PAPA/NO”), (Z)-1-(N-(3-aminopropyl)-N-(4-(3-aminopropylammonio)butyl)-amino) diazen-1-ium-1,2-diolate (spermine NONOate or “SPER/NO”) and sodium(Z)-1-(N,N-diethylamino)diazenium-1,2-diolate (diethylamine NONOate or “DEA/NO”) and derivatives thereof. NONOates are also described in U.S. Pat. Nos. 6,232,336, 5,910,316 and 5,650,447, the disclosures of which are incorporated herein by reference in their entirety. The “NO adducts” can be mono-nitrosylated, poly-nitrosylated, mono-nitrosated and/or poly-nitrosated at a variety of naturally susceptible or artificially provided binding sites for biologically active forms of nitrogen monoxide.

Suitable furoxanes include, but are not limited to, CAS 1609, C93-4759, C92-4678, S35b, CHF 2206, CHF 2363, and the like.

Suitable sydnonimines include, but are not limited to, molsidomine (N-ethoxycarbonyl-3-morpholinosydnonimine), SIN-1 (3-morpholinosydnonimine) CAS 936 (3-(cis-2,6-dimethylpiperidino)-N-(4-methoxybenzoyl)-sydnonimine, pirsidomine), C87-3754 (3-(cis-2,6-dimethylpiperidino)sydnonimine, linsidomine, C4144 (3-(3,3-dimethyl-1,4-thiazane-4-yl)sydnonimine hydrochloride), C89-4095 (3-(3,3-dimethyl-1,1-dioxo-1,4-thiazane-4-yl)sydnonimine hydrochloride, and the like.

Suitable oximes, include but are not limited to, NOR-1, NOR-3, NOR-4, and the like.

One group of NO adducts is the S-nitrosothiols, which are compounds that include at least one —S—NO group. These compounds include S-nitroso-polypeptides (the term “polypeptide” includes proteins and polyamino acids that do not possess an ascertained biological function, and derivatives thereof); S-nitrosylated amino acids (including natural and synthetic amino acids and their stereoisomers and racemic mixtures and derivatives thereof); S-nitrosylated sugars; S-nitrosylated, modified and unmodified, oligonucleotides (preferably of at least 5, and more preferably 5-200 nucleotides); straight or branched, saturated or unsaturated, aliphatic or aromatic, substituted or unsubstituted S-nitrosylated hydrocarbons; and S-nitroso heterocyclic compounds. S-nitrosothiols and methods for preparing them are described in U.S. Pat. Nos. 5,380,758 and 5,703,073; WO 97/27749; WO 98/19672; and Oae et al, Org. Prep. Proc. Int., 15(3):165-198 (1983), the disclosures of each of which are incorporated by reference herein in their entirety.

Another embodiment of the invention is S-nitroso amino acids where the nitroso group is linked to a sulfur group of a sulfur-containing amino acid or derivative thereof. Such compounds include, for example, S-nitroso-N-acetylcysteine, S-nitroso-captopril, S-nitroso-N-acetylpenicillamine, S-nitroso-homocysteine, S-nitroso-cysteine, S-nitroso-glutathione, S-nitroso-cysteinyl-glycine, and the like.

Suitable S-nitrosylated proteins include thiol-containing proteins (where the NO group is attached to one or more sulfur groups on an amino acid or amino acid derivative thereof) from various functional classes including enzymes, such as tissue-type plasminogen activator (TPA) and cathepsin B; transport proteins, such as lipoproteins; heme proteins, such as hemoglobin and serum albumin; and biologically protective proteins, such as immunoglobulins, antibodies and cytokines. Such nitrosylated proteins are described in WO 93/09806, the disclosure of which is incorporated by reference herein in its entirety. Examples include polynitrosylated albumin where one or more thiol or other nucleophilic centers in the protein are modified.

Other examples of suitable S-nitrosothiols include:

(i) HS(C(Rₑ)(R_{f}))ₘSNO;

(ii) ONS(C(Rₑ)(R_{f}))ₘRₑ; or

(iii) H₂N—CH(CO₂H)—(CH₂)ₘ—C(O)NH—CH(CH₂SNO)—C(O)NH—CH₂—CO₂H;

wherein m is an integer from 2 to 20;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, K or Rₑ and R_{f} taken together with the carbons to which they are attached form a carbonyl, a methanthial, a heterocyclic ring, a cycloalkyl group, an aryl group, an oxime, a hydrazone or a bridged cycloalkyl group;

K is —W₃)ₐ-E_{b}-(C(Rₑ)(R_{f}))ₚ₁-E_{c}-(C(Rₑ)(R_{f})ₓ—(W₃)_{d}—(C(Rₑ)(R_{f})_{y}—(W₃)ᵢ-Eⱼ-(W₃)_{g}—(C(Rₑ)(R_{f}))_{z}—U₃—V₃;

V₃ is —NO or —NO₂;

a, b, c, d, g, i and j are each independently an integer from 0 to 3;

p1, x, y and z are each independently an integer from 0 to 10;

W₃ at each occurrence is independently —C(O)—, —C(S)—, -T₃-, —(C(Rₑ)(R_{f})ₕ—, an alkyl group, an aryl group, a heterocyclic ring, an arylheterocyclic ring, or —(CH₂CH₂O)_{q1}—;

E at each occurrence is independently -T₃-, an alkyl group, an aryl group, —(C(Rₑ)(R_{f}))ₕ—, a heterocyclic ring, an arylheterocyclic ring, or —(CH₂CH₂O)_{q1}—;

T₃ at each occurrence is independently a covalent bond, a carbonyl, an oxygen, —S(O)ₒ— or —N(Rₐ)Rᵢ;

h is an integer form 1 to 10;

q₁ is an integer from 1 to 5;

U₃ at each occurrence is independently a covalent bond, a carbonyl, an oxygen, —S(O)ₒ— or —N(Rₐ)Rᵢ;

o is an integer from 0 to 2;

Rₐ is a lone pair of electrons, a hydrogen or an alkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U₃—V₃)(Rₑ)(R_{f}), a bond to an adjacent atom creating a double bond to that atom, —(N₂O₂—)⁻.M₁⁺, wherein M₁⁺ is an organic or inorganic cation.

In cases where Rₑ and R_{f} are a heterocyclic ring or taken together Rₑ and R_{f} are a heterocyclic ring, then Rᵢ can be a substituent on any disubstituted nitrogen contained within the radical wherein Rᵢ is as defined herein.

In cases where Rₑ and R_{f} are a heterocyclic ring or taken together Rₑ and R_{f} are a heterocyclic ring, then Rᵢ can be a substituent on any disubstituted nitrogen contained within the radical wherein Rᵢ is as defined herein.

Nitrosothiols can be prepared by various methods of synthesis. In general, the thiol precursor is prepared first, then converted to the S-nitrosothiol derivative by nitrosation of the thiol group with NaNO₂ under acidic conditions (pH is about 2.5) which yields the S-nitroso derivative. Acids which can be used for this purpose include aqueous sulfuric, acetic and hydrochloric acids. The thiol precursor can also be nitrosylated by reaction with an organic nitrite such as tert-butyl nitrite, or a nitrosonium salt such as nitrosonium tetrafluoroborate in an inert solvent.

Another group of NO adducts for use in the invention, where the NO adduct is a compound that donates, transfers or releases nitric oxide, include compounds comprising at least one ON—O— or ON—N— group. The compounds that include at least one ON—O— or ON—N— group are preferably ON—O— or ON—N-polypeptides (the term “polypeptide” includes proteins and polyamino acids that do not possess an ascertained biological function, and derivatives thereof); ON—O— or ON—N-amino acids (including natural and synthetic amino acids and their stereoisomers and racemic mixtures); ON—O— or ON—N-sugars; ON—O— or —ON—N— modified or unmodified oligonucleotides (comprising at least 5 nucleotides, preferably 5-200 nucleotides); ON—O— or ON—N— straight or branched, saturated or unsaturated, aliphatic or aromatic, substituted or unsubstituted hydrocarbons; and ON—O—, ON—N— or ON—C-heterocyclic compounds. Preferred examples of compounds comprising at least one ON—O— or ON—N— group include butyl nitrite, isobutyl nitrite, tert-butyl nitrite, amyl nitrite, isoamyl nitrite, N-nitrosamines, N-nitrosamides, N-nitrosourea, N-nitrosoguanidines, N-nitrosocarbamates, N-acyl-N-nitroso compounds (such as, N-methyl-N-nitrosourea); N-hydroxy-N-nitrosamines, cupferron, alanosine, dopastin, 1,3-disubstitued nitrosiminobenzimidazoles, 1,3,4-thiadiazole-2-nitrosimines, benzothiazole-2(3H)-nitrosimines, thiazole-2-nitrosimines, oligonitroso sydnonimines, 3-alkyl-N-nitroso-sydnonimines, 2H-1,3,4-thiadiazine nitrosimines.

Another group of NO adducts for use in the invention include nitrates that donate, transfer or release nitric oxide, such as compounds comprising at least one O₂N—O—, O₂N—N— or O₂N—S— group. Preferred among these compounds are O₂N—O—, O₂N—N— or O₂N—S— polypeptides (the term “polypeptide” includes proteins and also polyamino acids that do not possess an ascertained biological function, and derivatives thereof); O₂N—O—, O₂N—N— or O₂N—S— amino acids (including natural and synthetic amino acids and their stereoisomers and racemic mixtures); O₂N—O—, O₂N—N— or O₂N—S— sugars; O₂N—O—, O₂N—N— or O₂N—S— modified and unmodified oligonucleotides (comprising at least 5 nucleotides, preferably 5-200 nucleotides); O₂N—O—, O₂N—N— or O₂N—S— straight or branched, saturated or unsaturated, aliphatic or aromatic, substituted or unsubstituted hydrocarbons; and O₂N—O—, O₂N—N— or O₂N—S— heterocyclic compounds. Preferred examples of compounds comprising at least one O₂N—O—, O₂N—N— or O₂N—S— group include isosorbide dinitrate, isosorbide mononitrate, clonitrate, erythrityl tetranitrate, mannitol hexanitrate, nitroglycerin, pentaerythrtoltetranitrate, pentrinitrol, propatylnitrate and organic nitrates with a sulfhydryl-containing amino acid such as, for example SPM 3672, SPM 5185, SPM 5186 and those disclosed in U.S. Pat. Nos. 5,284,872, 5,428,061, 5,661,129, 5,807,847 and 5,883,122 and in WO 97/46521, WO 00/54756 and in WO 03/013432, the disclosures of each of which are incorporated by reference herein in their entirety.

Another group of NO adducts are N-oxo-N-nitrosoamines that donate, transfer or release nitric oxide and are represented by the formula: R^{1″}R^{2″}N—N(O—M⁺)—NO, where R^{1″} and R^{2″} are each independently a polypeptide, an amino acid, a sugar, a modified or unmodified oligonucleotide, a straight or branched, saturated or unsaturated, aliphatic or aromatic, substituted or unsubstituted hydrocarbon, or a heterocyclic group, and where M₁⁺ is an organic or inorganic cation, such, as for example, an alkyl substituted ammonium cation or a Group I metal cation.

The invention is also directed to compounds that stimulate endogenous NO or elevate levels of endogenous endothelium-derived relaxing factor (EDRF) in vivo or are oxidized to produce nitric oxide and/or are substrates for nitric oxide synthase and/or cytochrome P450. Such compounds include, for example, L-arginine, L-homoarginine, and N-hydroxy-L-arginine, N-hydroxy-L-homoarginine, N-hydroxydebrisoquine, N-hydroxypentamidine including their nitrosated and/or nitrosylated analogs (e.g., nitrosated L-arginine, nitrosylated L-arginine, nitrosated N-hydroxy-L-arginine, nitrosylated N-hydroxy-L-arginine, nitrosated and nitrosylated L-homoarginine), N-hydroxyguanidine compounds, amidoxime, ketoximes, aldoxime compounds, that can be oxidized in-vivo to produce nitric oxide. Compounds that may be substrates for a cytochrome P450, include, for example, imino(benzylamino)methylhydroxylamine, imino(((4-methylphenyl)methyl)amino)methylhydroxylamine, imino(((4-methoxyphenyl)methyl)amino) methylhydroxylamine, imino(((4-(trifluoromethyl)phenyl)methyl)amino) methylhydroxylamine, imino(((4-nitrophenyl)methyl)amino)methylhydroxylamine, (butylamino)iminomethylhydroxylamine, imino(propylamino) methylhydroxylamine, imino(pentylamino)methylhydroxylamine, imino(propylamino)methylhydroxylamine, imino ((methylethyl)amino)methylhydroxylamine, (cyclopropylamino)iminomethylhydroxylamine, imino-2-1,2,3,4-tetrahydroisoquinolyl methylhydroxylamine, imino(1-methyl(2-1,2,3,4-tetrahydroisoquinolyl))methylhydroxylamine, (1,3-methyl(2-1,2,3,4-tetrahydroisoquinolyl)) iminomethylhydroxylamine, (((4-chlorophenyl)methyl)amino)iminomethylhydroxylamine, ((4-chlorophenyl)amino) iminomethylhydroxylamine, (4-chlorophenyl)(hydroxyimino)methylamine, and 1-(4-chlorophenyl)-1-(hydroxyimino)ethane, and the like, precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid), nitric oxide mediators and/or physiologically acceptable salts thereof, including, for example, pyruvate, pyruvate precursors, α-keto acids having four or more carbon atoms, precursors of α-keto acids having four or more carbon atoms (as disclosed in WO 03/017996, the disclosure of which is incorporated herein in its entirety), and the substrates for nitric oxide synthase, cytokines, adenosin, bradykinin, calreticulin, bisacodyl, and phenolphthalein. EDRF is a vascular relaxing factor secreted by the endothelium, and has been identified as nitric oxide (NO) or a closely related derivative thereof (Palmer et al, Nature, 327:524-526 (1987); Ignarro et al, Proc. Natl. Acad. Sci. USA, 84:9265-9269 (1987)).

The invention is also based on the discovery that compounds and compositions of the invention may be used in conjunction with other therapeutic agents for co-therapies, partially or completely, in place of other therapeutic agents, such as, for example, including, but not limited to, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and combinations of two or more thereof. The therapeutic agent may optionally be nitrosated and/or nitrosylated.

In one embodiment of the invention, the therapeutic agents are β-adrenergic agonists, anti-allergic compounds, antitussive compounds, antioxidants, bronchodilators, expectorants, H₂ receptor antagonists, nonsteroidal antiinflammatory compounds (NSAIDs), phosphodiesterase inhibitors, proton pump inhibitors, selective cyclooxygenase-2 (COX-2) inhibitors, steroids, and combinations of two or more thereof.

Suitable aldosterone antagonists include, but are not limited to, canrenone, potassium canrenoate, drospirenone, spironolactone, eplerenone (INSPRA®), epoxymexrenone, fadrozole, pregn-4-ene-7,21-dicarboxylic acid, 9,11-epoxy-17-hydroxy-3-oxo, γ-lactone, methyl ester, (7α,11α,17β)-; pregn-4-ene-7,21-dicarboxylic acid, 9,11-epoxy-17-hydroxy-3-oxo-dimethyl ester, (7α,11α,17β)-; 3′H-cyclopropa(6,7)pregna-4,6-diene-21-carboxylic acid, 9,11-epoxy-6,7-dihydro-17-hydroxy-3-oxo-, γ-lactone, (6β,7β,11α,17 β)-; pregn-4-ene-7,21-dicarboxylic acid, 9,11-epoxy-17-hydroxy-3-oxo-, 7-(1-methylethyl)ester, monopotassium salt, (7α,11α,17β)-; pregn-4-ene-7,21-dicarboxylic acid, 9,11-epoxy-17-hydroxy-3-oxo-, 7-methyl ester, monopotassium salt, (7α,11α,17β)-; 3′H-cyclopropa(6,7) pregna-1,4,6-triene-21-carboxylic acid, 9,11-epoxy-6,7-dihydro-17-hydroxy-3-oxo-, γ-lactone, (6β,7β,11α)-; 3′H-cyclopropa(6,7)pregna-4,6-diene-21-carboxylic acid, 9,11-epoxy-6,7-dihydro-17-hydroxy-3-oxo-, methyl ester, (6β,7β,11α,17β)-; 3′H-cyclopropa(6,7)pregna-4,6-diene-21-carboxylic acid, 9,11-epoxy-6,7-dihydro-17-hydroxy-3-oxo-, monopotassium salt, (6β,7β,11α,17β)-; 3′H-cyclopropa(6,7)pregna-1,4,6-triene-21-carboxylic acid, 9,11-epoxy-6,7-dihydro-17-hydroxy-3-oxo-, γ-lactone, (6β,7β,11α,17β)-; pregn-4-ene-7,21-dicarboxylic acid, 9,11-epoxy-17-hydroxy-3-oxo-, γ-lactone, ethyl ester, (7α,11α,17β)-; pregn-4-ene-7,21-dicarboxylic acid, 9,11-epoxy-17-hydroxy-3-oxo-, γ-lactone, 1-methylethyl ester, (7α,11α,17β)-; RU-28318, and the like. Suitable aldosterone antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiment the aldosterone antagonists is eplerenone or spironolactone (a potassium sparing diuretic that acts like an aldosterone antagonist). In more particular embodiments eplerenone is administered in an amount of about 25 milligrams to about 300 milligrams as a single dose or as multiple doses per day; the spironolactone is administered in an amount of about 25 milligrams to about 150 milligrams as a single dose or as multiple doses per day.

Suitable alpha-adrenergic receptor antagonists include but are not limited to, phentolamine, tolazoline, idazoxan, deriglidole, RX 821002, BRL 44408, BRL 44409, BAM 1303, labetelol, ifenprodil, rauwolscine, corynathine, raubascine, tetrahydroalstonine, apoyohimbine, akuammigine, β-yohimbine, yohimbol, yohimbine, pseudoyohimbine, epi-3α-yohimbine, 10-hydroxy-yohimbine, 11-hydroxy-yohimbine, tamsulosin, benoxathian, atipamezole, BE 2254, WB 4101, HU-723, tedisamil, mirtazipine, setiptiline, reboxitine, delequamine, naftopil, saterinone, SL 89.0591, ARC 239, urapidil, 5-methylurapidil, monatepi, haloperidol, indoramin, SB 216469, moxisylyte, trazodone, dapiprozole, efaroxan, Recordati 15/2739, SNAP 1069, SNAP 5089, SNAP 5272, RS 17053, SL 89.0591, KMD 3213, spiperone, AH 11110A, chloroethylclonidine, BMY 7378, niguldipine, and the like. Suitable alpha-adrenergic receptor antagonists are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

Suitable β-adrenergic agonists include, but are not limited to, albuterol, bambuterol, bitolterol, carbuterol, clenbuterol, dobutamine, fenoterol, formoterol, hexoprenaline, isoprotenerol, mabuterol, metaproterenol, pirbuterol, prenalterol, procaterol, protokylol, ritodrine, rimiterol, reproterol, salmeterol, soterenol, terbutaline, tretoquinol, tulobuterol, and the like. Suitable β-adrenergic agonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable anti-allergic compounds include but are not limited to, acrivastine, allociamide, amlexanox, bromexine, cetirizine, clobenzepam, chromoglycate, chromolyn, deslortidine, emedastine, epinastine, fexofenadine, formoterol, hydroxyzine, ketotifen, loratadine, levocabastine, lodoxamide, mabuterol, montelukast, nedocromil, repirinast, salmeterol, seratrodast, suplatast tosylate, terfenadine, tiaramide, and the like. Suitable anti-allergic compounds are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable antidiabetic compounds include but are not limited to, acarbose, acetohexamide, buformin, carbutamide, chlorpropamide, glibornuride, gliclazide, glimepiride, glipizide, gliquidone, glisoxepid, glyburide, glybuthiazol(e), glybuzole, glyhexamide, glymidine, glypinamide, insulin, metformin, miglitol, nateglinide, phenbutamide, phenformin, pioglitazone, repaglinide, rosiglitazone, tolazamide, tolbutamide, tolcyclamide, troglitazone, voglibose, and the like. Suitable antidiabetic compounds are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

Suitable anti-hyperlipidemic compounds include, but are not limited to, statins or HMG-CoA reductase-inhibitors, such as, for example, atorvastatin (LIPITOR®), bervastatin, cerivastatin (BAYCOL®), dalvastatin, fluindostatin (Sandoz XU-62-320), fluvastatin, glenvastatin, lovastatin (MEVACOR®), mevastatin, pravastatin (PRAVACHOL®), rosuvastatin (CRESTRO®), simvastatin (ZOCOR®), velostatin (also known as synvinolin), VYTORIN™ (ezetimibe/simvastatin), GR-95030, SQ 33,600, BMY 22089, BMY 22,566, CI 980, and the like; gemfibrozil, cholystyramine, colestipol, niacin, nicotinic acid, bile acid sequestrants, such as, for example, cholestyramine, colesevelam, colestipol, poly(methyl-(3-trimethylaminopropyl)imino-trimethylene dihalide) and the like; probucol; fibric acid agents or fibrates, such as, for example, bezafibrate (Bezalip™), beclobrate, binifibrate, ciprofibrate, clinofibrate, clofibrate, etofibrate, fenofibrate (Lipidil™, Lipidil Micro™), gemfibrozil (Lopid™), nicofibrate, pirifibrate, ronifibrate, simfibrate, theofibrate and the like; cholesterol ester transfer protein (CETP) inhibitors, such as for example, CGS 25159, CP-529414 (torcetrapid), JTT-705, substituted N-[3-(1,1,2,2-tetrafluoroethoxy)benzyl]-N-(3-phenoxyphenyl)-trifluoro-3-amino-2-propanols, N,N-disubstituted trifluoro-3-amino-2-propanols, PD 140195 (4-phenyl-5-tridecyl-4H-1,2,4-triazole-3-thiol), SC-794, SC-795, SCH 58149, and the like.

In some embodiments the anti-hyperlipidemic compounds are atorvastatin, fluvastatin, lovastatin, pravastatin, rosuvastatin or simvastatin. In more particular embodiments the atorvastatin is administered in an amount of about 10 milligrams to about 80 milligrams as a single dose or as multiple doses per day; the fluvastatin is administered in an amount of about 20 milligrams to about 80 milligrams as a single does or as multiple doses per day; the lovastatin is administered in an amount of about 10 milligrams to about 80 milligrams as a single dose or as multiple doses per day; the pravastatin is administered in an amount of about 10 milligrams to about 80 milligrams as a single dose or as multiple doses per day; the rosuvastatin is administered in an amount of about 5 milligrams to about 40 milligrams as a single dose or as multiple doses per day; the simvastatin is administered in an amount of about 5 milligrams to about 80 milligrams as a single dose or as multiple doses per day.

Suitable antitussive compounds, include, but are not limited to, dextromethorphan, carbetapentane, caramiphen, diphenylhydramine, hydrocodene, codeine and the like. Suitable antitussive compounds are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable angiotensin II antagonists include, but are not limited to, angiotensin, abitesartan, candesartan, candesartan cilexetil, elisartan, embusartan, enoltasosartan, eprosartan, fonsartan, forasartan, glycyllosartan, irbesartan, losartan, olmesartan, milfasartan, medoxomil, ripisartan, pratosartan, saprisartan, saralasin, sarmesin, tasosartan, telmisartan, valsartan, zolasartan, 3-(2′ (tetrazole-5-yl)-1,1′-biphen-4-yl)methyl-5,7-dimethyl-2-ethyl-3H-imidazo(4,5-b)pyridine, antibodies to angiotensin II, A-81282, A-81988, BAY 106734, BIBR-363, BIBS-39, BIBS-222, BMS-180560, BMS-184698, BMS-346567, CGP-38560A, CGP42112A, CGP48369, CGP-49870, CGP-63170, CI-996, CP-148130, CL-329167, CV-11194, DA-2079, DE3489, DMP-811, DuP-167, DuP-532, DuP-753, E-1477, E-4177, E-4188, EMD-66397, EMD-666R4, EMD-73495, EMD-66684, EXP-063, EXP-929, EXP-3174, EXP-6155, EXP-6803, EXP-7711, EXP-9270, EXP-9954, FK-739, FRI 1153332, GA-0050, GA-0056, HN-65021, HOE-720, HR-720, ICI-D6888, ICI-D7155, ICI-D8731, KRI-1177, KT3-671, KT-3579, KW-3433, L-158809, L-158978, L-159282, L-159689, L-159874, L-161177, L-162154, L-162234, L-162441, L-163007, L-163017, LF-70156, LRB-057, LRB-081, LRB-087, LY-235656, LY-266099, LY-285434, LY-301875, LY-302289, LY-315995, ME3221, MK-954, PD-123177, PD-123319, PD-126055, PD-150304, RG-13647, RWJ-38970, RWJ46458, S-8307, S-8308, SC-51757, SC-54629, SC-52458, SC-52459, SK 1080, SL-910102, SR-47436, TAK-536, UP-2696, U-96849, U-97018, UK-77778, UP-275-22, WAY-126227, WK-1260, WK-1360, WK-1492, WY 126227, YH-1498, YM-358, YM-31472, X-6803, XH-148, XR-510, ZD-6888, ZD-7155, ZD-8731, ZD 8131, the compounds of ACS registry numbers 124750-92-1, 133240-46-7, 135070-05-2, 139958-16-0, 145160-84-5, 147403-03-0, 153806-29-2, 439904-54-8P, 439904-55-9P, 439904-56-0P, 439904-57-1P, 439904-58-2P, 155918-60-8P, 155918-61-9P, 272438-16-1P, 272446-75-0P, 223926-77-0P, 169281-89-4, 439904-65-1P, 165113-01-9P, 165113-02-0P, 165113-03-1P, 165113-03-2P, 165113-05-3P, 165113-064P, 165113-07-SP, 165113-08-6P, 165113-09-7P, 165113-10-0P, 165113-11-1P, 165113-12-2P, 165113-17-7P, 165113-18-8P, 165113-19-9P, 165113-20-2P, 165113-13-3P, 165113-14-4P, 165113-15-SP, 165113-16-6P, 165113-21-3P, 165113-22-4P, 165113-23-5P, 165113-24-6P, 165113-25-7P, 165113-26-8P, 165113-27-9P, 165113-28-0P, 165113-29-1P, 165113-30-4P, 165113-31-5P, 165113-32-6P, 165113-33-7P, 165113-34-8P, 165113-35-9P, 165113-36-0P, 165113-37-1P, 165113-38-2P, 165113-39-3P, 165113-40-6P, 165113-41-7P, 165113-42-8P, 1651134-37-9P, 165113-44-0P, 165113-45-1P, 165113-46-2P, 165113-47-3P, 165113-48-4P, 165113-49-5P, 165113-50-8P, 165113-51-9P, 165113-52-0P, 165113-53-1P, 165113-54-2P, 165113-55-3P, 165113-56-4P, 165113-57-5P, 165113-58-6P, 165113-59-7P, 165113-60-0P, 165113-61-1P, 165113-62-2P, 165113-63-3P, 165113-64-4P, 165113-65-5P, 165113-66-6P, 165113-67-7P, 165113-68-8P, 165113-69-9P, 165113-70-2P, 165113-71-3P, 165113-72-4P, 165113-73-SP, 165113-74-6P, 114798-27-5, 114798-28-6, 114798-29-7, 124749-82-2, 114798-28-6, 124749-844, 124750-88-5, 124750-91-0, 124750-93-2, 161946-65-2P, 16194747-3P, 161947484P, 161947-51-9P, 161947-52-0P, 161947-55-3P, 161947-564P, 161947-60-0P, 161947-61-1P, 161947-68-8P, 16194769-9P, 161947-70-2P, 161947-71-3P, 161947-724P, 161947-74-6P, 161947-75-7P, 161947-81-5P, 161947-82-6P, 161947-83-7P, 161947-84-8P, 161947-85-9P, 161947-86-0P, 161947-87-1P, 161947-88-2P, 161947-89-3P, 161947-90-6P, 161947-91-7P, 161947-92-8P, 161947-93-9P, 161947-94-0P, 161947-95-1P, 161947-96-2P, 161947-97-3P, 161947-98-4P, 161947-99-SP, 161948-00-P, 161948-01-2P, 161948-02-3P, 168686-32-6P, 16730142-0P, 166813-82-7P, 166961-56-4P, 166961-58-6P; 158872-96-9P, 158872-97-0P, 158807-14-8P, 158807-15-9P, 158807-16-0P, 158807-17-1P, 158807-18-2P, 158807-19-3P, 158807-20-6P, 155884-08-5P, 154749-99-2, 167371-59-7P, 244126-99-6P, 177848-35-0P and 141309-82-2P, and the like. Suitable angiotensin II antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments the angiotensin II antagonists are candesartan, eprosartan, irbesartan, losartan, omlesartan, telmisartan or valsartan. In more particular embodiments the candesartan is administered as candesartan cilexetil in an amount of about 15 milligrams to about 100 milligrams as a single dose or as multiple doses per day; the eprosartan, is administered as eprosartan mesylate in an amount of about 400 milligrams to about 1600 milligrams as a single does or as multiple doses per day; the irbesartan is administered in an amount of about 75 milligrams to about 1200 milligrams as a single dose or as multiple doses per day; the losartan is administered as losartan potassium in an amount of about 25 milligrams to about 100 milligrams as a single dose or as multiple doses per day; the omlesartan is administered as omlesartan medoxomil in an amount of about 5 milligrams to about 40 milligrams as a single dose or as multiple doses per day; the telmisartan is administered in an amount of about 20 milligrams to about 80 milligrams as a single dose or as multiple doses per day; the valsartan is administered in an amount of about 80 milligrams to about 320 milligrams as a single dose or as multiple doses per day.

Suitable angiotensin-converting enzyme inhibitors (ACE inhibitors) include, but are not limited to, alacepril, benazepril (LOTENSIN®, CIBACEN®), benazeprilat, captopril, ceronapril, cilazapril, delapril, duinapril, enalapril, enalaprilat, fasidotril, fosinopril, fosinoprilat, gemopatrilat, glycopril, idrapril, imidapril, lisinopril, moexipril, moveltipril, naphthopidil, omapatrilat, pentopril, perindopril, perindoprilat, quinapril, quinaprilat, ramipril, ramiprilat, rentipril, saralasin acetate, spirapril, temocapril, trandolapril, trandolaprilat, urapidil, zofenopril, acylmercapto and mercaptoalkanoyl pralines, carboxyalkyl dipeptides, carboxyalkyl dipeptide, phosphinylalkanoyl pralines, registry no. 796406, AVE 7688, BP1.137, CHF 1514, E 4030, ER 3295, FPL-66564, MDL 100240, RL 6134, RL 6207, RL 6893, SA 760, S-5590, Z 13752A, and the like. Suitable angiotensin-converting enzyme inhibitors are described more fully in the literature, such as in Goodman and Gilman, The Pharmacological Basis of Therapeutics (9th Edition), McGraw-Hill, 1995; and the Merck Index on CD-ROM, Twelfth Edition, Version 12:1, 1996; and on STN Express, file phar and file registry.

In some embodiments the angiotensin-converting enzyme inhibitors are benazepril, captopril, enalapril, fosinopril, lisinopril, moexipril, quinapril, ramipril, trandolapril or trandolaprilat. In more particular embodiments the benazepril is administered as benazepril hydrochloride in an amount of about 5 milligrams to about 80 milligrams as a single dose or as multiple doses per day; the captopril is administered in an amount of about 12.5 milligrams to about 450 milligrams as a single does or as multiple doses per day; the enalapril is administered as enalapril maleate in an amount of about 2.5 milligrams to about 40 milligrams as a single dose or as multiple doses per day; the fosinopril is administered as fosinopril sodium in an amount of about 5 milligrams to about 60 milligrams as a single dose or as multiple doses per day; the lisinopril is administered in an amount of about 12.5 milligrams to about 75 milligrams as a single dose or as multiple doses per day; the moexipril is administered as moexipril hydrochloride in an amount of about 7.5 milligrams to about 45 milligrams as a single dose or as multiple doses per day; the quinapril is administered as quinapril hydrochloride in an amount of about 5 milligrams to about 40 milligrams as single or multiple doses per day; the ramapril hydrochloride in an amount of about 1.25 milligrams to about 40 milligrams as single or multiple doses per day; the trandolapril is administered as in an amount of about 0.5 milligrams to about 4 milligrams as single or multiple doses per day; the trandolapril at is administered as in an amount of about 0.5 milligrams to about 4 milligrams as single or multiple doses per day.

Suitable antioxidants include, but are not limited to, small-molecule antioxidants and antioxidant enzymes. Suitable small-molecule antioxidants include, but are not limited to, hydralazine compounds, glutathione, vitamin C, vitamin E, cysteine, N-acetyl-cysteine, β-carotene, ubiquinone, ubiquinol-10, tocopherols, coenzyme Q, superoxide dismutase mimetics, such as, for example, 2,2,6,6-tetramethyl-1-piperidinyloxy (TEMPO), DOXYL, PROXYL nitroxide compounds; 4-hydroxy-2,2,6,6-tetramethyl-1-piperidinyloxy (Tempol), M-40401, M-40403, M-40407, M-40419, M-40484, M-40587, M-40588, and the like. Suitable antioxidant enzymes include, but are not limited to, superoxide dismutase, catalase, glutathione peroxidase, NADPH oxidase inhibitors, such as, for example, apocynin, aminoguanidine, ONO 1714, S17834 (benzo(b)pyran-4-one derivative), and the like; xanthine oxidase inhibitors, such as, for example, allopurinol, oxypurinol, amflutizole, diethyldithiocarbamate, 2-styrylchromones, chrysin, luteolin, kaempferol, quercetin, myricetin, isorhamnetin, benzophenones such as 2,2′,4,4′-tetrahydroxybenzophenone, 3,4,5,2′,3′,4′-hexahydroxybenzophenone and 4,4′-dihydroxybenzophenone; benzothiazinone analogues such as 2-amino-4H-1,3-benzothiazine-4-one, 2-guanidino-4H-1,3-benzothiazin-4-one and rhodanine; N-hydroxyguanidine derivative such as, PR5 (1-(3,4-dimethoxy-2-chlorobenzylideneamino)-3-hydroxyguanidine); 6-formylpterin, and the like. The antioxidant enzymes can be delivered by gene therapy as a viral vector and/or a non-viral vector. Suitable antioxidants are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the antioxidants are apocynin, hydralazine compounds and superoxide dimutase mimetics.

Suitable antithrombotic and vasodilator compounds include, but are not limited to, abciximab, acetorphan, acetylsalicylic acid, argatroban, bamethan, benfurodil, benziodarone, betahistine, bisaramil, brovincamine, bufeniode, citicoline, clobenfurol, clopidogrel, cyclandelate, dalteparin, dipyridamol, droprenilamine, enoxaparin, fendiline, ifenprodil, iloprost, indobufen, isobogrel, isoxsuprine, heparin, lamifiban, midrodine, nadroparin, nicotinoyl alcohol, nylidrin, ozagrel, perhexiline, phenylpropanolamine, prenylamine, papaveroline, reviparin sodium salt, ridogrel, suloctidil, tinofedrine, tinzaparin, trifusal, vintoperol, xanthinal niacinate, and the like. Suitable antithrombotic and vasodilator compounds are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

Suitable β-adrenergic antagonists include, but are not limited to, acebutolol, alprenolol, amosulalol, arotinolol, atenolol, befunolol, betaxolol, bevantolol, bisoprolol, bopindolol, bucindolol, bucumolol, bufetolol, bufuralol, bunitrolol, bupranolol, butofilolol, carazolol, capsinolol, carteolol, carvedilol (COREG®), celiprolol, cetamolol, cindolol, cloranolol, dilevalol, diprafenone, epanolol, ersentilide, esmolol, esprolol, hedroxalol, indenolol, labetalol, landiolol, laniolol, levobunolol, mepindolol, methylpranol, metindol, metipranolol, metrizoranolol, metoprolol, moprolol, nadolol, nadoxolol, nebivolol, nifenalol, nipradilol, oxprenolol, penbutolol, pindolol, practolol, pronethalol, propranolol, sotalol, sotalolnadolol, sulfinalol, taliprolol, talinolol, tertatolol, tilisolol, timolol, toliprolol, tomalolol, trimepranol, xamoterol, xibenolol, 2-(3-(1,1-dimethylethyl)-amino-2-hydroxypropoxy)-3-pyridenecarbonitrilHCl, 1-butylamino-3-(2,5-dichlorophenoxy)-2-propanol, 1-isopropylamino-3-(4(2-cyclopropylmethoxyethyl)phenoxy)-2-propanol, 3-isopropylamino-1-(7-methylindan-4-yloxy)-2-butanol, 2-(3-t-butylamino-2-hydroxy-propylthio)-4-(5-carbamoyl-2-thienyl)thiazol, 7-(2-hydroxy-3-t-butylaminpropoxy)phthalide, Acc 9369, AMO-140, BIB-16S, CP-331684, Fr-172516, ISV-208, L-653328, LM-2616, SB-226552, SR-58894A, SR-59230A, TZC-5665, UK-1745, YM-430, and the like. Suitable β-adrenergic antagonists are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments the β-adrenergic antagonists are atenolol, bisoprolol, carvedilol, metoprolol, nebivolol, propranolol or timolol. In more particular embodiments the atenolol is administered in an amount of about 50 milligrams to about 200 milligrams as a single dose or as multiple doses per day; the bisoprolol is administered as bisoprolol fumarate in an amount of about 2.5 milligrams to about 30 milligrams as a single dose or as multiple doses per day; the carvedilol is administered in an amount of about 3.125 milligrams to about 200 milligrams as a single does or as multiple doses per day; the metoprolol is administered as metoprolol tartarate in an amount of about 50 milligrams to about 300 milligrams as a single dose or as multiple doses per day; the nebivolol is administered as nebivolol hydrochloride in an amount of about 2.5 milligrams to about 20 milligrams as a single dose or as multiple doses per day; the propranolol is administered as propranolol hydrochloride in an amount of about 40 milligrams to about 240 milligrams as a single dose or as multiple doses per day; the timolol is administered as timolol maleate in an amount of about 10 milligrams to about 30 milligrams as a single dose or as multiple doses per day.

Suitable bronchodilators include but are not limited to, ambroxol, atropine, bevonium methyl sulfate, bethanechol, chlorprenaline, cyclodrine, daiphenacine, N-desethyl-oxybutynin, dicyclomine, emepronium, ephedrine, epinephrine, etafredine, ethylnorepinephrine, flavoxate, flutoprium bromide, hexoprenaline, 2-hydroxy-2,2-diphenyl-N-(1,2,3,6-tetrahydro-pyridin-4-ylmethyl)acetamide, ipratropium bromide, isoetharine, NS 21, oxybutynin, oxitropium bromide, propanthelin, propiverine, rispenzepine, terbutaline, 1-teobromine actetic acid, terodiline, tiotropium bromide, tolterodine, trospium, vamicamide, zamiphenacine, and the like. Suitable bronchodilators are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable calcium channel blockers include, but are not limited to, amlodipine (NORVASC®), anipamil, aranidipine, amrinone, azelnidipine, barnidipine, bencyclane, benidipine, bepridil, cilnidipine, cinnarizine, clentiazem, diltiazem, dotarizine, efonidipine, elgodipine, fantofarone, felodipine, fendiline, flunarizine, fluspirilene, furnidipine, gallopamil, ipenoxazone, isradipine, lacidipine, lemildipine, lercanidipine, lomerizine, manidipine, mibefradil, monatepil, nicardipine, nifedipine, niguldipine, niludipine, nilvadipine, nimodipine, nisoldipine, nitrendipine, nivaldipine, oxodipine, perhexilene, phenytoin, phenytprenylamine, pranidipine, ranolazine, ryosidine, semotiadil, tamolarizine, temiverine hydrochloride, terodiline, tiapamil, vatanidipine hydrochloride, verapamil, ziconotide, AE-0047, CAI, JTV-519, CHF-1521, L-651582, NS-7, NW-1015, RO-2933, SB-237376, SL-34.0829-08, S-312d, SD-3212, TA-993, YM-430, and the like. Suitable calcium channel blockers are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the calcium channel blockers are amlodipine, diltiazem, isradipine, nicardipine, nifedipine, nimodipine, nisoldipine, nitrendipine, verapamil.

Suitable diuretics include but are not limited to, thiazides (such as, for example, althiazide, bendroflumethiazide, benzclortriazide, benzhydrochlorothiazide, benzthiazide, buthiazide, chlorothiazide, cyclopenethiazide, cyclothiazide, epithiazide, ethiazide, hydrobenzthiazide, hydrochlorothiazide, hydroflumethiazide, methylclothiazide, methylcyclothiazide, penflutazide, polythiazide, teclothiazide, trichlormethiazide, triflumethazide, and the like); alilusem, ambuside, amiloride, aminometradine, azosemide, bemetizide, bumetanide, butazolamide, butizide, canrenone, carperitide, chloraminophenamide, chlorazanil, chlormerodrin, chlorthalidone, cicletanide, clofenamide, clopamide, clorexolone, conivaptan, daglutril, dichlorophenamide, disulfamide, ethacrynic acid, ethoxzolamide, etozolon, fenoldopam, fenquizone, furosemide, indapamide, mebutizide, mefruside, meralluride, mercaptomerin sodium, mercumallylic acid, mersalyl, methazolamide, meticane, metolazone, mozavaptan, muzolimine, N-(5-1,3,4-thiadiazol-2-yl)acetamide, nesiritide, pamabrom, paraflutizide, piretanide, protheobromine, quinethazone, scoparius, spironolactone, theobromine, ticrynafen, torsemide, torvaptan, triamterene, tripamide, ularitide, xipamide or potassium, AT 189000, AY 31906, BG 9928, BG 9791, C 2921, DTI 0017, JDL 961, KW 3902, MCC 134, SLV 306, SR 121463, WAY 140288, ZP 120, and the like. Suitable diuretics are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Depending on the diuretic employed, potassium may also be administered to the patient in order to optimize the fluid balance while avoiding hypokalemic alkalosis. The administration of potassium can be in the form of potassium chloride or by the daily ingestion of foods with high potassium content such as, for example, bananas or orange juice. The method of administration of these compounds is described in further detail in U.S. Pat. No. 4,868,179, the disclosure of which is incorporated by reference herein in its entirety.

In some embodiments the diuretics are amiloride, furosemide, chlorthalidone, hydrochlorothiazide or triamterene. In more particular embodiments the amiloride is administered as amiloride hydrochloride in an amount of about 5 milligrams to about 15 milligrams as a single dose or as multiple doses per day; the furosemide is administered in an amount of about 10 milligrams to about 600 milligrams as a single does or as multiple doses per day; the chlorthalidone is administered in an amount of about 15 milligrams to about 150 milligrams as a single dose or as multiple doses per day; the hydrochlorothiazide is administered in an amount of about 12.5 milligrams to about 300 milligrams as a single dose or as multiple doses per day; the triamterene is administered in an amount of about 35 milligrams to about 225 milligrams as a single dose or as multiple doses per day.

Suitable endothelin antagonists include, but are not limited to, atrasentan, bosentan, darusentan, endothelin, enrasentan, sitaxsentan, sulfonamide endothelin antagonists, tezosentan, BMS 193884, BQ-123, SQ 28608, and the like. Suitable endothelin antagonists are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

Suitable expectorants include, but are not limited to, ambroxol, domiodol, erdosteine, guaiacol, guaifenesin, iodinated glycerol, letosteine, mensa, sobrerol, strepronine, terpin, tiopronin, and the like. Suitable expectorants are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Suitable hydralazine compounds include, but are not limited to, compounds having the formula:

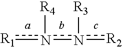

wherein a, b and c are independently a single or double bond; R₁ and R₂ are each independently a hydrogen, an alkyl, an ester or a heterocyclic ring, wherein alkyl, ester and heterocyclic rind are as defined herein; R₃ and R₄ are each independently a lone pair of electrons or a hydrogen, with the proviso that at least one of R₁, R₂, R₃ and R₄ is not a hydrogen. Exemplary hydralazine compounds include budralazine, cadralazine, dihydralazine, endralazine, hydralazine, pildralazine, todralazine, and the like. Suitable hydralazine compounds are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the hydralazine compound is hydralazine or a pharmaceutically acceptable salt thereof such as hydralazine hydrochloride. In more particular embodiments the hydralazine is administered as hydralazine hydrochloride in an amount of about 10 milligrams to about 300 milligrams as a single dose or as multiple doses per day.

Suitable H₂ receptor antagonists include, but are not limited to, burimamide, cimetidine, ebrotidin, famotidine, nizatidine, roxatidine, rantidine, tiotidine, and the like. Suitable H₂ receptor antagonists are described more fully in the literature, such as in Goodman and Gilman, The Pharmacological Basis of Therapeutics (9th Edition), McGraw-Hill, 1995, Pgs. 901-915; the Merck Index on CD-ROM, 13^{th} Edition; and in WO 00/28988 assigned to NitroMed Inc., the disclosures of which are incorporated herein by reference in their entirety.

Suitable neutral endopeptidase inhibitors include, but are not limited to, atrial natriuretic peptides, diazapins, azepinones, ecadotril, fasidotril, fasidotrilat, omapatrilat, sampatrilat, BMS 189,921, Z 13752 A, and the like. Neutral endopeptidase inhibitors are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

Suitable NSAIDs include, but are not limited to, acetaminophen, acemetacin, aceclofenac, alminoprofen, amfenac, bendazac, benoxaprofen, bromfenac, bucloxic acid, butibufen, carprofen, cinmetacin, clopirac, diclofenac, etodolac, felbinac, fenclozic acid, fenbufen, fenoprofen, fentiazac, flunoxaprofen, flurbiprofen, ibufenac, ibuprofen, indomethacin, isofezolac, isoxepac, indoprofen, ketoprofen, lonazolac, loxoprofen, metiazinic acid, mofezolac, miroprofen, naproxen, oxaprozin, pirozolac, pirprofen, pranoprofen, protizinic acid, salicylamide, sulindac, suprofen, suxibuzone, tiaprofenic acid, tolmetin, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, meclofenamic acid, mefenamic acid, mesalamine, prodrugs thereof, and the like. Suitable NSAIDs are described more fully in the literature, such as in Goodman and Gilman, The Pharmacological Basis of Therapeutics (9th Edition), McGraw-Hill, 1995, Pgs. 617-657; the Merck Index on CD-ROM, 13^{th} Edition; and in U.S. Pat. Nos. 6,057,347 and 6,297,260 assigned to NitroMed Inc., the disclosures of which are incorporated herein by reference in their entirety.

In some embodiments the NSAIDs are acetaminophen, diclofenac, flurbiprofen, ibuprofen, indomethacin, ketoprofen, naproxen or aspirin. In more particular embodiments the acetaminophen is administered in an amount of about 325 milligrams to about 4 grams as a single dose or as multiple doses per day-, the diclofenac is administered in an amount of about 50 milligrams to about 250 milligrams as a single does or as multiple doses per day; the flurbiprofen is administered in an amount of about 100 milligrams to about 300 milligrams as a single does or as multiple doses per day; the ibuprofen is administered in an amount of about 400 milligrams to about 3.2 grams as a single does or as multiple doses per day; the indomethacin is administered in an amount of about 25 milligrams to about 200 milligrams as a single does or as multiple doses per day; the ketoprofen is administered in an amount of about 50 milligrams to about 300 milligrams as a single does or as multiple doses per day; the naproxen is administered in an amount of about 250 milligrams to about 1.5 grams as a single does or as multiple doses per day; the aspirin is administered in an amount of about 10 milligrams to about 2 grams as a single does or as multiple doses per day.

Suitable phosphodiesterase inhibitors, include but are not limited to, filaminast, piclamilast, rolipram, Org 20241, MCI-154, roflumilast, toborinone, posicar, lixazinone, zaprinast, sildenafil, pyrazolopyrimidinones, motapizone, pimobendan, zardaverine, siguazodan, CI 930, EMD 53998, imazodan, saterinone, loprinone hydrochloride, 3-pyridinecarbonitrile derivatives, acefylline, albifylline, bamifylline, denbufyllene, diphylline, doxofylline, etofylline, torbafylline, theophylline, nanterinone, pentoxofylline, proxyphylline, cilostazol, cilostamide, MS 857, piroximone, milrinone, amrinone, tolafentrine, dipyridamole, papaveroline, E4021, thienopyrimidine derivatives, triflusal, ICOS-351, tetrahydropiperazino(1,2-b)beta-carboline-1,4-ione derivatives, carboline derivatives, 2-pyrazolin-5-one derivatives, fused pyridazine derivatives, quinazoline derivatives, anthranilic acid derivatives, imidazoquinazoline derivatives, tadalafil, vardenafil, and in Goodman and Gilman, The Pharmacological Basis of Therapeutics (9th Ed.), McGraw-Hill, Inc. (1995), The Physician's Desk Reference (49th Ed.), Medical Economics (1995), Drug Facts and Comparisons (1993 Ed), Facts and Comparisons (1993), and the Merck Index on CD-ROM, 13^{th} Edition; and the like. Phosphodiesterase inhibitors and their nitrosated and/or nitrosylated derivatives are also disclosed in U.S. Pat. Nos. 5,932,538, 5,994,294, 5,874,437, 5,958,926 reissued as U.S. Pat. Nos. RE 0,377,234 6,172,060, 6,197,778, 6,177,428, 6,172,068, 6,221,881, 6,232,321, 6,197,782, 6,133,272, 6,211,179, 6,316,457 and 6,331,542, the disclosures of each of which are incorporated herein by reference in their entirety.

Suitable potassium channel blockers include but are not limited to, nicorandil, pinacidil, cromakalim (BRL 34915), aprikalim, bimakalim, emakalim, lemakaim, minoxidil, diazoxide, 9-chloro-7-(2-chlorophenyl)-5H-pyrimido(5,4,-d)(2)-benzazepine, Ribi, CPG-11952, CGS-9896, ZD 6169, diazixide, Bay X 9227, P1075, Bay X 9228, SDZ PCO 400, WAY-120,491, WAY-120,129, Ro 31-6930, SR 44869, BRL 38226, S 0121, SR 46142A, CGP 42500, SR 44994, artilide fumarate, lorazepam, temazepam, rilmazafone, nimetazepam, midazolam, lormetazepam, loprazolam, ibutilide fumarate, haloxazolam, flunitrazepam, estazolam, doxefazepam, clonazepam, cinolazepam, brotizolam, and the like. Suitable potassium channel blockers are described more fully in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

Suitable platelet reducing agents include but are not limited to, fibrinolytic agents such as for example, ancrod, anistreplase, bisobrin lactate, brinolase, Hageman factor (i.e. factor XII) fragments, plasminogen activators such as, for example, streptokinase, tissue plasminogen activators (TPA), urokinase, pro-urokinase, recombinant TPA, plasmin, plasminogen, and the like; anti-coagulant agents including but are not limited to, inhibitors of factor Xa, factor TFPI, factor VIIa, factor IXc, factor Va, factor VIIIa, inhibitors of other coagulation factors, and the like; vitamin K antagonists, such as, for example, coumarin, coumarin derivatives (e.g., warfarin sodium); glycosoaminoglycans such as, for example, heparins both in unfractionated form and in low molecular weight form; ardeparin sodium, bivalirudin, bromindione, coumarin, dalteparin sodium, danaparoid sodium; dazoxiben hydrochloride, desirudin, dicumarol, efegatran sulfate, enoxaparin sodium, ifetroban, ifetroban sodium, lyapolate sodium, nafamostat mesylate, phenprocoumon, sulfatide, tinzaparin sodium, retaplase; trifenagrel, warfarin, dextrans and the like; abciximab, acadesine, anipamil, argatroban, aspirin, clopidogrel, diadenosine 5′,5′″-P1,P4-tetraphosphate (Ap4A) analogs, difibrotide, dilazep dihydrochloride, dipyridamole, dopamine, 3-methoxytyramine, glucagon, glycoprotein IIb/IIIa antagonists, such as, for example, Ro-43-8857, L-700,462, iloprost, isocarbacyclin methyl ester, itazigrel, ketanserin, BM-13.177, lamifiban, lifarizine, molsidomine, nifedipine, oxagrelate, prostaglandins, platelet activating factor antagonists such as, for example, lexipafant, prostacyclins, pyrazines, pyridinol carbamate, ReoPro (i.e., abciximab), sulfinpyrazone, synthetic compounds BN-50727, BN-52021, CV-4151, E-5510, FK-409, GU-7, KB-2796, KBT-3022, KC-404, KF-4939, OP-41483, TRK-100, TA-3090, TFC-612, ZK-36374, 2,4,5,7-tetrathiaoctane, 2,4,5,7-tetrathiaoctane 2,2-dioxide, 2,4,5-trithiahexane, theophyllin pentoxifyllin, thromboxane and thromboxane synthetase inhibitors such as, for example, picotamide, sulotroban, ticlopidine, tirofiban, trapidil, ticlopidine, trifenagrel, trilinolein, 3-substituted 5,6-bis(4-methoxyphenyl)-1,2,4-triazines; antibodies to glycoprotein IIb/IIIa; anti-serotonin drugs, such as, for example, clopridogrel; sulfinpyrazone and the like; aspirin; dipyridamole; clofibrate; pyridinol carbamate; glucagon, caffeine; theophyllin pentoxifyllin; ticlopidine, and the like.

Suitable proton pump inhibitors include, but are not limited to, disulprazole, esomeprazole, lansoprazole, leminoprazole, omeprazole, pantoprazole, rabeprazole, timoprazole, tenatoprazole, 2-(2-benzimidazolyl)-pyridine, tricyclic imidazole, thienopydidine benzimidazole, fluoroalkoxy substituted benzimidazole, dialkoxy benzimidazole, N-substituted 2-(pyridylalkenesulfinyl)benzimidazole, cycloheptenepyridine, 5-pyrrolyl-2-pyridylmethylsulfinyl benzimidazole, alkylsulfinyl benzimidazole, fluoro-pyridylmethylsulfinyl benzimidazole, imidazo(4,5-b)pydridine, RO 18-5362, IY 81149, 4-amino-3-carbonyl quinoline, 4-amino-3-acylnaphthyride, 4-aminoquinoline, 4-amino-3-acylquinoline, 3-butyryl-4-(2-methylphenylamino)-8-(2-hydroxyethoxy)quinoline, quinazoline, tetrahydroisoquinolin-2-yl pyrimidine, YH 1885, 3-substituted 1,2,4-thiadiazolo(4,5-a)benzimidazole, 3-substituted imidazo(1,2-d)-thiadiazole, 2-sulfinylnicotinamide, pyridylsulfinylbenz imidazole, pyridylsulfinyl thieno imidazole, theinoimidazole-toluidine, 4,5-dihydrooxazole, thienoimidazole-toluidine, Hoe-731, imidazo(1,2-a)pyridine, pyrrolo(2,3-b)pyridine, and the like. Suitable proton pump inhibitors are described more fully in the literature, such as in Goodman and Gilman, The Pharmacological Basis of Therapeutics (9th Edition), McGraw-Hill, 1995; the Merck Index on CD-ROM, 13^{th} Edition; and in WO 00/50037 assigned to NitroMed Inc., the disclosures of which are incorporated herein by reference in their entirety.

Suitable renin inhibitors include, but are not limited to, aldosterone, aliskiren (SPP-100), ditekiren, enalkrein (A-64662), medullipin, terlkiren, tonin, zankiren, RO 42-5892 (remikiren), A 62198, A 64662, A 65317, A 69729, A 72517 (zankiren), A 74273, CP 80794, CGP 29287, CGP-38560A, EMD 47942, ES 305, ES 1005, ES 8891, FK 906, FK 744, H 113, H-142, KRI 1314, pepstatin A, RO 44-9375 (ciprokiren), RO 42-5892, RO 66-1132, RO 66-1168, SP 500, SP 800, SR-43845, SQ 34017, U 71038, YM-21095, YM-26365, urea derivatives of peptides, amino acids connected by nonpeptide bonds, di- and tri-peptide derivatives (e.g., Act-A, Act-B, Act-C, ACT-D, and the like), amino acids and derivatives thereof, diol sulfonamides and sulfinyls, modified peptides, peptidyl beta-aminoacyl aminodiol carbamates, monoclonal antibodies to renin. Suitable renin inhibitors are described more fully in U.S. Pat. Nos. 5,116,835, 5,114,937, 5,106,835, 5,104,869, 5,095,119, 5,098,924), 5,095,006, 5,089,471, 5,075,451, 5,066,643, 5,063,208, 4,845,079, 5,055,466, 4,980,283, 4,885,292), 4,780,401, 5,071,837, 5,064,965, 5,063,207, 5,036,054, 5,036,053, 5,034,512, and 4,894,437, the disclosures of each of which are incorporated herein by reference in their entirety; and in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

Suitable COX-2 inhibitors include, but are not limited to, nimesulide, celecoxib (CELEBREX®), etoricoxib (ARCOXIA®), flosulide, lumiracoxib (PREXIG®, COX-189), parecoxib (DYNSTAT®), rofecoxib (VIOXX®), tiracoxib (JTE-522), valdecoxib (BEXTRA®), ABT 963, BMS 347070, CS 502, DuP 697, GW-406381, NS-386, SC-57666, SC-58125, SC-58635, and the like, and combinations of two or more thereof. Suitable COX-2 inhibitors are in U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, 5,639,780, 5,932,598 and 6,633,272, and in WO 94/03387, WO 94/15723, WO 94/20480, WO 94/26731, WO 94/27980, WO 95/00501, WO 95/15316, WO 96/03387, WO 96/03388, WO 96/06840, WO 96/21667, WO 96/31509, WO 96/36623, WO 97/14691, WO 97/16435, WO 01/45703 and WO 01/87343, the disclosures of each of which are incorporated herein by reference in their entirety; and in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the COX-2 inhibitors are celecoxib, etoracoxib, lumiracoxib, paracoxib, rofecoxib or valdecoxib. In more particular embodiments the celecoxib is administered in an amount of about 100 milligrams to about 800 milligrams as a single dose or as multiple doses per day; the etoricoxib is administered in an amount of about 50 milligrams to about 200 milligrams as a single does or as multiple doses per day; the lumiracoxib is administered in an amount of about 40 milligrams to about 1200 milligrams as a single dose or as multiple doses per day; the paracoxib is administered in an amount of about 20 milligrams to about 100 milligrams as a single does or as multiple doses per day; the rofecoxib is administered in an amount of about 12.5 milligrams to about 50 milligrams as a single does or as multiple doses per day; the valdecoxib is administered in an amount of about 10 milligrams to about 40 milligrams as a single does or as multiple doses per day;

Suitable steroids, include but are not limited to, 21-acetoxypregnenolone, alcolometasone, algestone, amcinonide, beclomethasone, betamethasone, budesonide, chloroprednisone, cidesamide, clobetasol, clobetasone, clocortolone, cloprednol, corticosterone, cortisone, cortivazol (cortivatol), dchenodeoxycholic acid, eflazacort, desonide, desoxycorticosterone, desoximethasone, dexamethasone, diflorasone, diflucortolone, difluprednate, enoxolone, estradiol, ethynylestradiol, fluzacort, fludrocortisone, flucloronide, flumethasone, flunisolide, flucinolone acetonide, fluocinonide, fluocortin butyl, fluocortolone, fluorometholone, fluperolone acetate, fluprednidene acetate, flu prednisolone, flurandrenolide, fluticasone propionate, formocortal, halcinonide, halobetasol propionate, halometasone, haloprednone acetate, hydrocortamate, hydrocortisone and its derivatives (such as phosphate, 21-sodium succinate and the like), hydrocortisone terbutate, isoflupredone, loteprednol etabonate, mestranol, mazipredone, medrysone, meprednisone, methylprednisolone, mitatrienediol, mometasone furoate, moxestrol, paramethasone, prednicarbate, prednisolone and its derivatives (such as 21-stearoylglycolate, sodium phosphate, 25-diethylaminoacetate, and the like), prednisone, prednival, prednylidene and its derivatives (such as 21-diethylaminoactetate and the like), rimexolone, tixocortol, triamcinolone and its derivatives (such as acetonide, benetonide, and the like), ursodeoxycholic acid, and the like. Suitable steroids are described more fully in the literature, such as in Goodman and Gilman, The Pharmacological Basis of Therapeutics (9th Edition), McGraw-Hill, 1995; the Merck Index on CD-ROM, 13^{th} Edition; the disclosures of which are incorporated herein by reference in their entirety.

Another embodiment of the invention provides methods for treating bacterial infections by administering to the patient in need thereof a therapeutically effective amount of the compounds and/or compositions described herein. For example, the patient can be administered a therapeutically effective amount of at least one nitrosated and/or nitrosylated compound of the invention. In another embodiment, the patient can be administered a therapeutically effective amount of at least one compound of the invention, that is optionally nitrosated and/or nitrosylated, and at least one nitric oxide donor compound. In yet another embodiment, the patient can be administered a therapeutically effective amount of at least one compound of the invention, that is optionally nitrosated and/or nitrosylated, and, at least one therapeutic agent, including but not limited to, such as, for example, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and, optionally, at least one nitric oxide donor compound. The compounds of the invention, that are optionally nitrosated and/or nitrosylated, nitric oxide donors, and/or therapeutic agents can be administered separately or as components of the same composition in one or more pharmaceutically acceptable carriers.

In one embodiment the invention provides methods for treating bacterial infections associated with pulmonary infections in patients with disease including, but not limited to, endobronchial infections, cystic fibrosis, bronchiectasis, pneumonia, tuberculosis, emphysema, AIDS, pneumoccal meningitis, bacteremia, otitis media, chronic obstructive pulmonary disease, sinus congestion, common cold, septicemia and the like; gastrointestinal infections, including, but not limited to, chronic gastritis, gastric ulcer, duodenal ulcer, Helicobacter pylori, gastric malignant lymphoma, gastroenteritis, diarrhea, dysentery, inflammatory bowel disease, Crohn's disease, ulcerative colitis, infections resulting from E. Coli, and the like; and infections of the eyes, ear or nose, by administering to the patient in need thereof a therapeutically effective amount of the compounds and/or compositions described herein. In one embodiment, the invention provides methods for treating cystic fibrosis. For example, the patient can be administered a therapeutically effective amount of at least one nitrosated and/or nitrosylated compound of the invention. In another embodiment, the patient can be administered a therapeutically effective amount of at least one compound of the invention, that is optionally nitrosated and/or nitrosylated, and at least one nitric oxide donor compound. In yet another embodiment, the patient can be administered a therapeutically effective amount of at least one compound of the invention, that is optionally nitrosated and/or nitrosylated, and, at least one therapeutic agent, including but not limited to, such as, for example, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and, optionally, at least one nitric oxide donor compound. The compounds of the invention, that are optionally nitrosated and/or nitrosylated, nitric oxide donors, and/or therapeutic agents can be administered separately or as components of the same composition in one or more pharmaceutically acceptable carriers.

Another embodiment of the invention provides methods for treating viral infections by administering to the patient in need thereof a therapeutically effective amount of the compounds and/or compositions described herein. For example, the patient can be administered a therapeutically effective amount of at least one nitrosated and/or nitrosylated compound of the invention. In another embodiment, the patient can be administered a therapeutically effective amount of at least one compound of the invention, that is optionally nitrosated and/or nitrosylated, and at least one nitric oxide donor compound. In yet another embodiment, the patient can be administered a therapeutically effective amount of at least one compound of the invention, that is optionally nitrosated and/or nitrosylated, and, at least one therapeutic agent, including but not limited to, such as, for example, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and, optionally, at least one nitric oxide donor compound. The compounds of the invention, that are optionally nitrosated and/or nitrosylated, nitric oxide donors, and/or therapeutic agents can be administered separately or as components of the same composition in one or more pharmaceutically acceptable carriers.

Yet another embodiment of the invention provides methods for treating fungal infections by administering to the patient in need thereof a therapeutically effective amount of the compounds and/or compositions described herein. For example, the patient can be administered a therapeutically effective amount of at least one nitrosated and/or nitrosylated compound of the invention. In another embodiment, the patient can be administered a therapeutically effective amount of at least one compound of the invention, that is optionally nitrosated and/or nitrosylated, and at least one nitric oxide donor compound. In yet another embodiment, the patient can be administered a therapeutically effective amount of at least one compound of the invention, that is optionally nitrosated and/or nitrosylated, and, at least one therapeutic agent, including but not limited to, such as, for example, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and, optionally, at least one nitric oxide donor compound. The compounds of the invention, that are optionally nitrosated and/or nitrosylated, nitric oxide donors, and/or therapeutic agents can be administered separately or as components of the same composition in one or more pharmaceutically acceptable carriers.

Yet another embodiment of the invention provides methods for treating lesions by administering to the patient in need thereof a therapeutically effective amount of the compounds and/or compositions described herein. For example, the patient can be administered a therapeutically effective amount of at least one nitrosated and/or nitrosylated compound of the invention. In another embodiment, the patient can be administered a therapeutically effective amount of at least one compound of the invention, that is optionally nitrosated and/or nitrosylated, and at least one nitric oxide donor compound. In yet another embodiment, the patient can be administered a therapeutically effective amount of at least one compound of the invention, that is optionally nitrosated and/or nitrosylated, and, at least one therapeutic agent, including but not limited to, such as, for example, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, β-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and, optionally, at least one nitric oxide donor compound. The compounds of the invention, that are optionally nitrosated and/or nitrosylated, nitric oxide donors, and/or therapeutic agents can be administered separately or as components of the same composition in one or more pharmaceutically acceptable carriers.

When administered separately, the compound of the invention, that is optionally nitrosated and/or nitrosylated, nitric oxide donor and/or therapeutic agent can be administered about the same time as part of the overall treatment regimen, i.e., as a combination therapy. “About the same time” includes administering the compound, that is optionally nitrosated and/or nitrosylated, simultaneously, sequentially, at the same time, at different times on the same day, or on different days, as long as they are administered as part of an overall treatment regimen, i.e., combination therapy or a therapeutic cocktail.

When administered in vivo, the compounds and compositions of the invention can be administered in combination with pharmaceutically acceptable carriers and in dosages described herein. When the compounds and compositions of the invention are administered as a combination of at least one compound of the invention and/or at least one nitrosated and/or nitrosylated compound of the invention and/or at least one nitric oxide donor and/or therapeutic agent, they can also be used in combination with one or more additional compounds which are known to be effective against the specific disease state targeted for treatment. The nitric oxide donors, therapeutic agents and/or other additional compounds can be administered simultaneously with, subsequently to, or prior to administration of the nitrosated and/or nitrosylated compound of the invention.

The compounds and compositions of the invention can be administered by any available and effective delivery system including, but not limited to, orally, bucally, parenterally, by inhalation, by topical application, by injection, transdermally, or rectally (e.g., by the use of suppositories) in dosage unit formulations containing conventional nontoxic pharmaceutically acceptable carriers, adjuvants, and vehicles, as desired. Parenteral includes subcutaneous injections, intravenous, intramuscular, intrasternal injection, or infusion techniques.

Transdermal compound administration, which is known to one skilled in the art, involves the delivery of pharmaceutical compounds via percutaneous passage of the compound into the systemic circulation of the patient. Topical administration can also involve the use of transdermal administration such as transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, sprays, lotions, gels, ointments, eye drops, nose drops, ear drops, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous crosslinking agent impregnated with the composition and laminated to an impermeable backing.

The compositions can also be applied topically using a transdermal system, such as one of an acrylic-based polymer adhesive with a resinous crosslinking agent impregnated with the composition and laminated to an impermeable backing. In a particular embodiment, the compositions of the invention are administered as a transdermal patch, more particularly as a sustained-release transdermal patch. The transdermal patches of the invention can include any conventional form such as, for example, adhesive matrix, polymeric matrix, reservoir patch, matrix or monolithic-type laminated structure, and are generally comprised of one or more backing layers, adhesives, penetration enhancers, an optional rate controlling membrane and a release liner which is removed to expose the adhesives prior to application. Polymeric matrix patches also comprise a polymeric-matrix forming material. Suitable transdermal patches are described in more detail in, for example, U.S. Pat. Nos. 5,262,165, 5,948,433, 6,010,715 and 6,071,531, the disclosure of each of which are incorporated herein in their entirety.

Solid dosage forms for oral administration can include capsules, sustained-release capsules, tablets, sustained release tablets, chewable tablets, sublingual tablets, effervescent tablets, pills, powders, granules and gels. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Suppositories for vaginal or rectal administration of the compounds and compositions of the invention, such as for treating pediatric fever and the like, can be prepared by mixing the compounds or compositions with a suitable nonirritating excipient such as cocoa butter and polyethylene glycols which are solid at room temperature but liquid at rectal temperature, such that they will melt in the rectum and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium.

Inhaled formulations can be administered, for example, as pressurized aerosols and/or nebulized formulations to the patient's lungs. Such formulations may contain a variety of known aerosol propellants useful for endopulmonary and/or intranasal inhalation administration. In addition, water may be present, with or without any of a variety of cosolvents, surfactants, stabilizers (such as, for example, antioxidants, chelating agents, inert gases, buffers and the like). The formulation may also be aerosolized by atomizing which can produce aerosols and/or dry powder particles between 1 and 5 microns for the efficacious delivery of the inhaled formulation.

The compositions of this invention can further include conventional excipients, i.e., pharmaceutically acceptable organic or inorganic carrier substances suitable for parenteral application which do not deleteriously react with the active compounds. Suitable pharmaceutically acceptable carriers include, for example, water, salt solutions, alcohol, vegetable oils, polyethylene glycols, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers.

The composition, if desired, can also contain minor amounts of wetting agents, emulsifying agents and/or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and the like.

Various delivery systems are known and can be used to administer the compounds or compositions of the invention, including, for example, encapsulation in liposomes, microbubbles, emulsions, microparticles, microcapsules and the like. The required dosage can be administered as a single unit or in a sustained release form.

The bioavailability of the compositions can be enhanced by micronization of the formulations using conventional techniques such as grinding, milling, spray drying and the like in the presence of suitable excipients or agents such as phospholipids or surfactants.

Sustained release dosage forms of the invention may comprise microparticles and/or nanoparticles having a therapeutic agent dispersed therein or may comprise the therapeutic agent in pure, preferably crystalline, solid form. For sustained release administration, microparticle dosage forms comprising pure, preferably crystalline, therapeutic agents are preferred. The therapeutic dosage forms of this aspect of the invention may be of any configuration suitable for sustained release.

Nanoparticle sustained release therapeutic dosage forms are preferably biodegradable and, optionally, bind to the vascular smooth muscle cells and enter those cells, primarily by endocytosis. The biodegradation of the nanoparticles occurs over time (e.g., 30 to 120 days; or 10 to 21 days) in prelysosomic vesicles and lysosomes. Preferred larger microparticle therapeutic dosage forms of the invention release the therapeutic agents for subsequent target cell uptake with only a few of the smaller microparticles entering the cell by phagocytosis. A practitioner in the art will appreciate that the precise mechanism by which a target cell assimilates and metabolizes a dosage form of the invention depends on the morphology, physiology and metabolic processes of those cells. The size of the particle sustained release therapeutic dosage forms is also important with respect to the mode of cellular assimilation. For example, the smaller nanoparticles can flow with the interstitial fluid between cells and penetrate the infused tissue. The larger microparticles tend to be more easily trapped interstitially in the infused primary tissue, and thus are useful to deliver anti-proliferative therapeutic agents.

Particular sustained release dosage forms of the invention comprise biodegradable microparticles or nanoparticles. More particularly, biodegradable microparticles or nanoparticles are formed of a polymer containing matrix that biodegrades by random, nonenzymatic, hydrolytic scissioning to release therapeutic agent, thereby forming pores within the particulate structure.

In a particular embodiment, the compositions of the invention are administered by inhalation. For example, the inhaled formulations can comprise a therapeutically effective amount of at least one nitrosated and/or nitrosylated compound of the invention or a pharmaceutically acceptable salt thereof, and, optionally at least one nitric oxide donor, or the inhaled formulations can comprise a therapeutically effective amount of at least one nitrosated and/or nitrosylated compound of the invention or a pharmaceutically acceptable salt thereof, and at least one nitric oxide donor, and, optionally at least one therapeutic agent

The compounds and compositions of the invention can be formulated as pharmaceutically acceptable salt forms. Pharmaceutically acceptable salts include, for example, alkali metal salts and addition salts of free acids or free bases. The nature of the salt is not critical, provided that it is pharmaceutically-acceptable. Suitable pharmaceutically-acceptable acid addition salts may be prepared from an inorganic acid or from an organic acid. Examples of such inorganic acids include, but are not limited to, hydrochloric, hydrobromic, hydroiodic, nitric, carbonic, sulfuric and phosphoric acid and the like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, β-hydroxybutyric, cyclohexylaminosulfonic, galactaric and galacturonic acid and the like. Suitable pharmaceutically-acceptable base addition salts include, but are not limited to, metallic salts made from aluminum, calcium, lithium, magnesium, potassium, sodium and zinc or organic salts made from primary, secondary and tertiary amines, cyclic amines, N,N′-dibenzylethylenediamine, chloroprocaine, choline, diethanolamine, ethylenediamine, meglumine (N-methylglucamine) and procaine and the like. All of these, salts may be prepared by conventional means from the corresponding compound by reacting, for example, the appropriate acid or base with the compound.

While individual needs may vary, determination of optimal ranges for effective amounts of the compounds and/or compositions is within the skill of the art. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

The amount of a given nitrosated and/or nitrosylated compound of the invention that will be effective in the treatment of a particular disorder or condition will depend on the nature of the disorder or condition, and can be determined by standard clinical techniques, including reference to Goodman and Gilman, supra; The Physician's Desk Reference, Medical Economics Company, Inc., Oradell, N.J., 1995; and Drug Facts and Comparisons, Inc., St. Louis, Mo., 1993. The precise dose to be used in the formulation win also depend on the route of administration, and the seriousness of the disease or disorder, and should be decided by the physician and the patient's circumstances. For example, in one embodiment a nitrosated and/or nitrosylated compound of the invention is administered at about 2.5 mg to 1 gram, once a day or multiple times per day.

The invention also provides pharmaceutical kits comprising one or more containers filled with one or more of the ingredients of the pharmaceutical compounds and/or compositions of the invention, including, at least, one or more of the novel compound of the invention, that is optionally nitrosated and/or nitrosylated, and one or more of the NO donors described herein. Associated with such kits can be additional therapeutic agents or compositions (e.g., including, but not limited to, aldosterone antagonists, alpha-adrenergic receptor antagonists, β-adrenergic agonists, anti-allergic compounds, antidiabetic compounds, anti-hyperlipidemic drugs, antitussive compounds, angiotensin II antagonists, angiotensin-converting enzyme (ACE) inhibitors, antioxidants, antithrombotic and vasodilator drugs, p-adrenergic antagonists, bronchodilators, calcium channel blockers, diuretics, endothelin antagonists, expectorants, hydralazine compounds, H₂ receptor antagonists, neutral endopeptidase inhibitors, nonsteroidal antiinflammatory compounds (NSAIDs), phosphodiesterase inhibitors, potassium channel blockers, platelet reducing agents, proton pump inhibitors, renin inhibitors, selective cyclooxygenase-2 (COX-2) inhibitors, steroids, and combinations of two or more thereof) devices for administering the compositions, and notices in the form prescribed by a governmental agency regulating the manufacture, use or sale of pharmaceuticals or biological products which reflects approval by the agency of manufacture, use or sale for humans.

The disclosure of each patent, patent application and publication cited or described in the present specification is hereby incorporated by reference herein in its entirety.

Although the invention has been set forth in detail, one skilled in the art will appreciate that numerous changes and modifications can be made to the invention, and that such changes and modifications can be made without departing from the spirit and scope of the invention.

1. A nitrosated antimicrobial compound, a nitrosated adenosine antagonist, a nitrosated LTB4 antagonist, a nitrosated mucoregulator, a nitrosated purine agonist or a pharmaceutically acceptable salt thereof, having at least one nitrosated carboxylic acid group (—C(O)X), nitrosated hydroxyl group (—OX), nitrosated thiol group (—SX) and/or primary or secondary nitrosated amine group (—NX); wherein the nitrosated antimicrobial compound is a nitrosated acediasulfone, a nitrosated aceturate, a nitrosated acetyl sulfametossipirazine, a nitrosated acetyl sulfamethoxypyrazine, a nitrosated acranil, a nitrosated albendazole, a nitrosated alexidine, a nitrosated amatadine, a nitrosated ambazone, an a nitrosated amdinocillin, a nitrosated amikacin, a nitrosated p-aminosalicylic acid, a nitrosated p-aminosalicylic acid hydrazine, a nitrosated amoxicillin, a nitrosated ampicillin, a nitrosated anisomycin, a nitrosated apalcillin, a nitrosated apicyclin, a nitrosated apramycin, a nitrosated arbekacin, a nitrosated argininsa, a nitrosated aspoxicillin, a nitrosated azidamfenicol, a nitrosated azidocillin, a nitrosated azithromycin, a nitrosated azlocillin, a nitrosated aztreonam, a nitrosated bacampicillin, a nitrosated benzoylpas, a nitrosated benzyl penicillin acid, a nitrosated benzyl sulfamide, a nitrosated bicozamycin, a nitrosated bipenam, a nitrosated brodimoprim, a nitrosated capreomycin, a nitrosated carbenicillin, a nitrosated carbomycin, a nitrosated cafazedone, a nitrosated carindacillin, a nitrosated carumonam, a nitrosated cefcapene pivoxil, a nitrosated cefaclor, a nitrosated cefadroxil, a nitrosated cefafroxil, a nitrosated cefamandole, a nitrosated cefatamet, a nitrosated cefatrizine, a nitrosated cefazedone, a nitrosated cefazolin, a nitrosated cefbuperazone, a nitrosated cefclidin, a nitrosated cefdinir, a nitrosated cefditoren, a nitrosated cefixime, a nitrosated cefmenoxime, a nitrosated cefmetazole, a nitrosated cefminox, a nitrosated cefodizime, a nitrosated cefonicid, a nitrosated cefoperazone, a nitrosated ceforanide, a nitrosated cefotaxime, a nitrosated cefotetan, a nitrosated cefotiam, a nitrosated cefoxitin, a nitrosated cefozopran, a nitrosated cefpimizole, a nitrosated cefpiramide, a nitrosated cefpirome, a nitrosated cefpodoxime proxetil, a nitrosated cefprozil, a nitrosated cefroxadine, a nitrosated cefsulodin, a nitrosated ceftazidime, a nitrosated cefteram, a nitrosated ceftezole, a nitrosated ceftibuten, a nitrosated ceftiofur, a nitrosated ceftizoxime, a nitrosated ceftriaxone, a nitrosated cefuroxime, a nitrosated cefuzonam, a nitrosated cephacetrile sodium, a nitrosated cephadrine, a nitrosated cephalexin, a nitrosated cepbaloglycin, a nitrosated cephaloridine, a nitrosated cephalosporin C, a nitrosated cephalothin, a nitrosated cephapirin sodium, a nitrosated cephradine, a nitrosated chloramphenicol, a nitrosated chlorotetracycline, a nitrosated cinoxacin, a nitrosated ciprofloxacin, a nitrosated claritromycin, a nitrosated clavulanic acid, a nitrosated clinafloxacin, a nitrosated clindamycin, a nitrosated clofazimine, a nitrosated clofoctal, a nitrosated clometocillin, a nitrosated clomocycline, a nitrosated cloxacillin, a nitrosated cloxyquin, a nitrosated cyclacilline, a nitrosated cycloserine, a nitrosated danoflaxcin, a nitrosated dapsone, a nitrosated deoxycycline, a nitrosated deoxydihydrostreptomycin, a nitrosated dibekacin, a nitrosated dicloxacillin, a nitrosated difloxacin, a nitrosated dihydrostreptomycin, a nitrosated dimetridazole, a nitrosated diminazene, a nitrosated dirirtomycin, a nitrosated eflornithine, a nitrosated enrofloxacin, a nitrosated enviomycin, a nitrosated epicillin, a nitrosated erythromycin, a nitrosated etacillin, a nitrosated ethambutol, a nitrosated ethionamide, a nitrosated famcyclovir, a nitrosated fenbecillin, a nitrosated fleroxacin, a nitrosated flomoxef, a nitrosated floxacillin, a nitrosated flumequine, a nitrosated furonazide, a nitrosated fortimycin, a nitrosated furazolium, a nitrosated gentamycin, a nitrosated glyconiazide, a nitrosated grepafloxacin, a nitrosated guamecycline, a nitrosated halofuginone, a nitrosated hetacillin, a nitrosated homidium, a nitrosated hydroxyl-stilbamidine, a nitrosated ibostamycin, a nitrosated imidocarb, a nitrosated imipenam, a nitrosated ipronidazole, a nitrosated isoniazide, a nitrosated josamycin, a nitrosated inosine, a nitrosated lauroguadine, a nitrosated lenampicillin, a nitrosated lincomycin, a nitrosated lomefloxacin, a nitrosated loracarbef, a nitrosated lymecyclin, a nitrosated mafenide, a nitrosated mebendazole, a nitrosated meclocyclin, a nitrosated meropenem, a nitrosated metampicillin, a nitrosated metacicline, a nitrosated methacycline, a nitrosated methicillin, a nitrosated metronidazole, a nitrosated 4′-(methylsulfamoyl)sulfanilanilide, a nitrosated mezlocillin, a nitrosated meziocillin, a nitrosated micronomycin, a nitrosated midecamycin A₁, a nitrosated minocycline, a nitrosated miocamycin, a nitrosated miokamycin, a nitrosated morfazinamide, a nitrosated moxalactam, a nitrosated mupirocin, a nitrosated myxin, a nitrosated nadifloxacin, a nitrosated nalidixic acid, a nitrosated negamycin, a nitrosated neomycin, a nitrosated netlimycin, a nitrosated nifurfoline, a nitrosated nifurpirinol, a nitrosated nifurprazine, a nitrosated nimorazole, a nitrosated nitroxoline, a nitrosated norfloxacin, a nitrosated novobiocin, a nitrosated ofloxacin, a nitrosated oleandomycin, a nitrosated opiniazide, a nitrosated oxacillin, a nitrosated oxophenarsine, a nitrosated oxolinic acid, a nitrosated oxytetracycline, a nitrosated panipenam, a nitrosated paromycin, a nitrosated pazufloxacin, a nitrosated pefloxacin, a nitrosated penicillin G, a nitrosated penicillin N, a nitrosated penicillin O, a nitrosated penicillin V, a nitrosated penethamate hydroiodide, a nitrosated pentamidine, a nitrosated phenamidine, a nitrosated phenethicillin, a nitrosated phenyl aminosalicyclate, a nitrosated pipacycline, a nitrosated pipemidic acid, a nitrosated piperacillin, a nitrosated pirlimycin, a nitrosated piromidic acid, a nitrosated pivampicillin, a nitrosated pivcefalexin, a nitrosated profiromycin, a nitrosated propamidine, a nitrosated propicillin, a nitrosated protionamide, a nitrosated puraltadone, a nitrosated puromycin, a nitrosated pyrazinamide, a nitrosated pyrimethamine, a nitrosated quinacillin, a nitrosated quinacrine, a nitrosated quinapyramine, a nitrosated quintine, a nitrosated ribostamycin, a nitrosated rifabutine, a nitrosated rifamide, a nitrosated rifampin, a nitrosated rifamycin, a nitrosated rifanpin, a nitrosated rifapentine, a nitrosated rifaxymine, a nitrosated ritipenem, a nitrosated rokitamycin, a nitrosated rolitetracycline, a nitrosated rosamycin, a nitrosated rufloxacin, a nitrosated salazosulfadimidine, a nitrosated salinazid, a nitrosated sancycline, a nitrosated sarafloxacin, a nitrosated sedacamycin, a nitrosated secnidazole, a nitrosated sisomycin, a nitrosated sparfloxacin, a nitrosated spectinomycin, a nitrosated spiramycin, a nitrosated spiramycin I, a nitrosated spiramycin II, a nitrosated spiramycin III, a nitrosated stilbamidine, a nitrosated streptomycin, a nitrosated streptonicizid, a nitrosated sulbactam, a nitrosated sulbenicillin, a nitrosated succisulfone, a nitrosated sulfanilamide, a nitrosated sulfabenzamide, a nitrosated sulfacetamide, a nitrosated sulfachloropyridazine, a nitrosated sulfachrysoidine, a nitrosated sulfacytine, a nitrosated sulfadiazine, a nitrosated sulfadicramide, a nitrosated sulfadimethoxine, a nitrosated sulfadoxine, a nitrosated sulfadrazine, a nitrosated sulfaetidol, a nitrosated sulfafenazol, a nitrosated sulfaguanidine, a nitrosated sulfaguanole, a nitrosated sulfalene, a nitrosated sulfamerazine, a nitrosated sulfameter, a nitrosated sulfamethazine, a nitrosated sulfamethizole, a nitrosated sulfamethomidine, a nitrosated sulfamethoxazole, a nitrosated sulfamethoxypyridazine, a nitrosated sulfamethyltiazol, a nitrosated sulfamethylthiazole, a nitrosated sulfametrole, a nitrosated sulfamidochrysoidine, a nitrosated sulfamoxole, a nitrosated sulfanilamide, a nitrosated 4-sulfanilamido salicylic acid, a nitrosated 4-4′-sulfanilylbenzylamine, a nitrosated p-sulfanilylbenzylamine, a nitrosated 2-p-sulfinylanilinoethanol, a nitrosated sulfanilylurea, a nitrosated sulfoniazide, a nitrosated sulfaperine, a nitrosated sulfaphenazole, a nitrosated sulfaproxyline, a nitrosated sulfapyrazine, a nitrosated sulfapyridine, a nitrosated sulfathiazole, a nitrosated sulfaethidole, a nitrosated sulfathiourea, a nitrosated sulfisomidine, a nitrosated sulfasomizole, a nitrosated sulfasymazine, a nitrosated sulfisoxazole, 4,4′-sulfinyldianiline, a nitrosated N⁴-sulfanilylsulfanilamide, a nitrosated N-sulfanilyl-3,4-xylamide, a nitrosated sultamicillin, a nitrosated talampicillin, a nitrosated tambutol, a nitrosated taurolidine, a nitrosated teiclplanin, a nitrosated temocillin, a nitrosated tetracycline, a nitrosated tetroxoprim, a nitrosated thiabendazole, a nitrosated thiazolsulfone, a nitrosated tibezonium, a nitrosated ticarcillin, a nitrosated tigemonam, a nitrosated tinidazole, a nitrosated tobramycin, a nitrosated tosufloxacin, a nitrosated trimethoprim, a nitrosated troleandromycin, a nitrosated a nitrosated trospectomycin, a nitrosated trovafloxacin, a nitrosated tubercidine, a nitrosated miokamycin, a nitrosated oleandomycin, a nitrosated troleandromycin, a nitrosated vancomycin, a nitrosated verazide, a nitrosated viomycin, a nitrosated virginiamycin, a nitrosated zalcitabine, a nitrosated acyclovir, a nitrosated amatadine, a nitrosated cidofovir, a nitrosated cytarabine, a nitrosated didanosine, a nitrosated dideoxyadenosine, a nitrosated edoxudine, a nitrosated famciclovir, a nitrosated floxuridine, a nitrosated gancyclovir, a nitrosated idoxuridine, a nitrosated indanavir, a nitrosated kethoxal, a nitrosated lamivudine, a nitrosated MADU, a nitrosated penciclovir, a nitrosated podophyllotoxin, a nitrosated ribavirine, a nitrosated rimantadine, a nitrosated saquinavir, a nitrosated sorivudine, a nitrosated stavudine, a nitrosated trifluridine, a nitrosated valacyclovir, a nitrosated vidarabine, a nitrosated xenazoic acid, a nitrosated zalcitabine, a nitrosated zidovudine, a nitrosated daptomycin, a nitrosated duramycin, a nitrosated nafcillin, a nitrosated tigecycline, a nitrosated PA-1806, or a nitrosated PA-2794; the nitrosated adenosine agonist is a nitrosated CPX; the nitrosated LTB4 antagonist is a nitrosated amelubant; the nitrosated mucoregulator is a nitrosated talniflumate, a nitrosated MSI-2216, a nitrosated ML-03, or a nitrosated INO-4995; the nitrosated purine agonist is a nitrosated P2Y2 agonist, a nitrosated INS-37217, a nitrosated uridine 5′triphosphate or a nitrosated diquafosol tetrasodium;
wherein X is:
(1) —Y—(CR₄R₄′)ₚ-T-(CR₄R₄′)ₚ—ONO₂; 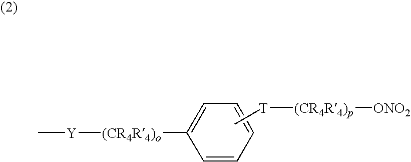
wherein T is ortho, meta or para; 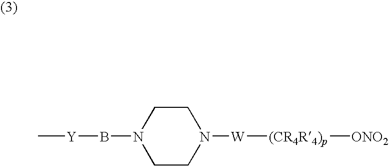
(4) —Y—(CR₄C₄′)ₚ—V—B-T-(CR₄R₄′)ₚ—ONO₂;
(5) —Y—(CR₄R₄′)ₚ-T-C(O)—(CR₄R₄′)ₒ—(CH₂)—ONO₂;
(6) —Y—(CR₄R₄′)ₚ—C(Z)-(CH₂)_{q}-T-(CR₄R₄′)_{q}—(CH₂)—ONO₂;
(7) —Y—(CR₄R₄′)ₚ-T-(CH₂)_{q}—V—(CR₄R₄′)_{q}—(CH₂)—ONO₂;
(8) —Y—(CR₄R₄′)ₚ—V—(CH₂)_{q}—V—(CR₄R₄′)_{q}—(CH₂)—ONO₂;
(9) —Y—(CR₄R₄′)ₒ—(W)_{q}—(CR₄R₄′)ₒ—(CH₂)—ONO₂;
(10) —NRⱼ—O—(CH₂)ₒ—V—(CR₄R₄′)_{q}—(CH₂)—ONO₂;
(11) —NRⱼ—O—(CH₂)ₒ—(W)_{q}—(CR₄R₄′)_{q}—(CH₂)—ONO₂;
(12) —O—NRⱼ—(CH₂)ₒ—(W)_{q}—(CR₄R₄′)_{q}—(CH₂)—ONO₂;
(13) —Y—(CH₂)ₒ—(W)_{q}—(CH₂)ₒ—V—(CR₄R₄′)ₒ-Q′-(CR₄R₄′)ₒ—(CH₂)—ONO₂;
(14) —Y—(CR₄R₄′)ₚ—V—(CH₂)ₒ—(W)_{q}—(CR₄R₄′)_{q}—(CH₂)—ONO₂;
(15) —O—NRⱼ—(CH₂)ₒ—V—(CR₄R₄′)_{q}—(CH₂)—ONO₂;
(16) —Y—(CR₄R₄′)ₒ-Q′-(CR₄R₄′)ₒ—V—(CR₄R₄′)ₒ—(CH₂)—ONO₂;
(17) —Y—(CR₄R₄′)ₒ-Q′-(CR₄R₄′)ₒ—(W)_{q}—(CR₄R₄′)ₒ—(CH₂)—ONO₂;
(18) —Y—(CR₄R₄′)ₚ-T-(CR₄R₄′)ₚ-Q′-(CR₄R₄′)ₒ—(CH₂)—ONO₂;
(19) —Y—(CR₄R₄′)_{q}—C(Z)-(CR₄R₄′)ₒ—(CH₂)—ONO₂;
(20) —Y—(CR₄R₄′)ₚ-Q′-(CR₄R₄′)ₒ—(CH₂)—ONO₂;
(21) —Y—(CR₄R₄′)_{q}—P(O)MM′;
(22) —Y—(CR₄R₄′)ₒ-Q′-(CR₄R₄′)ₒ—(CH₂)—ONO₂;
(23) —Y—(CR₄R₄′)ₒ-Q′-(CR₄R₄′)ₒ-T-(CR₃R₄′), —(CH₂)—ONO₂;
(24) —Y—(CR₄R₄′)_{q}—(W)_{q}—(CR₄R₄′)ₒ-Q′-(CR₄R₄′)ₒ—(CH₂)—ONO₂;
(25) —Y—(CR₄R₄′)_{q}—V—(CR₄R₄′)ₒ-Q′-(CR₄R₄′)ₒ—(CH₂)—ONO₂;
(26) —Y—(CR₄R₄′)ₚ-(T)ₒ-(W)_{q}—(CR₄R₄′)ₒ—(CH₂)—ONO₂;
(27) —Y—(CR₄R₄′)ₚ—(W)_{q}-(T)ₒ-(CR₄R₄′)—(CH₂)—ONO₂;
(28) —Y—(CR₄R₄′)_{q}—C(Z)-V—(CR₄R₄′)_{q}—(CH₂)—ONO₂;
(29) —Y—(CR₄R₄′)_{O}—C(R₄)(ONO₂)—(CR₄R₄′)_{q}-(T)ₒ-(W)_{q}-(T)ₒ-(CR₄R₄′)ₒ—R₅;
(30) —Y—(CR₄R₄′)ₒ—V—(CR₄R₄′)ₒ-Q′-(CR₄R₄′)—(CH₂)—ONO₂;
(31) —Y—(CR₄R₄′)_{q}—C(Z)-Q′-(CR₄R₄′)ₒ—(CH₂)—ONO₂;
(32) —Y—(CR₄R₄′)ₚ—V—(CR₄R₄′)ₚ—(CH₂)—ONO₂;
(33) —Y—(CR₄R₄′)ₚ—V—(CH₂)_{q}-(T)ₒ-(CR₄R₄′)_{q}—(CH₂)—ONO₂;
(34) —Y—(CR₄R₄′)ₚ-(T)ₒ-Q′-(T)ₒ-(CR₄R₄′)_{q}—(CH₂)—ONO₂;
(35) —Y—(CR₄R₄′)_{q}—C(Z)-(CR₄R₄′)_{q}—V—(CR₄R₄′)ₒ-Q′-(CR₄R₄′)ₒ—(CH₂)—ONO₂;
(36) —Y—(CR₄R₄′)_{q}—C(Z)-(CR₄R₄′)_{q}—(W)_{q}—(CR₄R₄′)ₒ-Q′-(CR₄R₄′)ₒ—(CH₂)—ONO₂;
(37) —NRⱼ—O—(CH₂)ₒ—V—(CR₄R₄′)ₒ-Q′-(CH₂)—ONO₂;
(38) —NRⱼ—O—(CH₂)ₒ—(W)_{q}—(CR₄R₄′)ₒ-Q′-(CH₂)—ONO₂;
(39) —O—NRⱼ—(CH₂)ₒ—(W)_{q}—(CR₄R₄′)ₒ-Q′-(CH₂)—ONO₂;
(40) —O—NRⱼ—(CH₂)ₒ—V—(CR₄R₄′)ₒ-Q′-(CH₂)—ONO₂;
(41) —NRⱼ—NRⱼ—(CR₄R₄′)ₚ—(W)_{q}-(T)ₒ-(CR₄R₄′)ₒ—(CH₂)—ONO₂; or
(42) —Y—(CR₄R₄′)ₒ-Q′-(CR₄R₄′)ₒ—ONO₂; or
(43) —Y—(CR₄R₄′)ₒ—V—(CR₄R₄′)ₒ-Q-(CR₄R₄′)ₒ—ONO₂;
R₄ and R₄′ at each occurrence are independently a hydrogen, lower alkyl group, —OH, —CH₂OH, —ONO₂, —NO₂ or —CH₂ONO₂; or R₄ and R₄′ taken together with the carbon atom to which they are attached are a cycloalkyl group or a heterocyclic ring;
V is —C(O)-T-, -T-C(O)—, -T-C(O)-T or T-C(O)—C(O)-T;
W is a covalent bond or a carbonyl group;
T at each occurrence is independently an oxygen, (S(O)ₒ)ₒ or NRⱼ;
Rⱼ is a hydrogen, an alkyl group, an aryl group, a heterocyclic ring, an alkylcarbonyl group, an alkylaryl group, an alkylsulfinyl group, an alkylsulfonyl group, an arylsulfinyl group, an arylsulfonyl group, a sulfonamido group, a N-alkylsulfonamido group, a N,N-diarylsulfonamido group, a N-arylsulfonamido group, a N-alkyl-N-arylsulfonamido group, a carboxamido group or a hydroxyl group;
p at each occurrence is independently an integer from 1 to 6;
q at each occurrence is independently an integer from 1 to 3;
o at each occurrence is independently an integer from 0 to 2;
Y is independently a covalent bond, a carbonyl, an oxygen, —S(O)ₒ— or —NRⱼ;
B is either phenyl or (CH₂)ₒ;
Q′ is a cycloalkyl group, a heterocyclic ring or an aryl group;
Z is (═O), (═N—OR₅), (═N—NR₅R′₅) or (═CR₅R′₅);
M and M′ are each independently —O⁻ H₃N⁺—(CR₄R′₄)_{q}—CH₂ONO₂ or -T-(CR₄R′₄)ₒ—CH₂ONO₂; and
R₅ and R₅′ at each occurrence are independently a hydrogen, a hydroxyl group, an alkyl group, an aryl group, an alkylsulfonyl group, an arylsulfonyl group, a carboxylic ester, an alkylcarbonyl group, an arylcarbonyl group, a carboxamido group, an alkoxyalkyl group, an alkoxyaryl group, a cycloalkyl group or a heterocyclic ring.

2. The nitrosated antimicrobial compound, the nitrosated adenosine antagonist, the a nitrosated LTB4 antagonist, the nitrosated mucoregulator, the nitrosated purine agonist of claim 1, wherein X is: 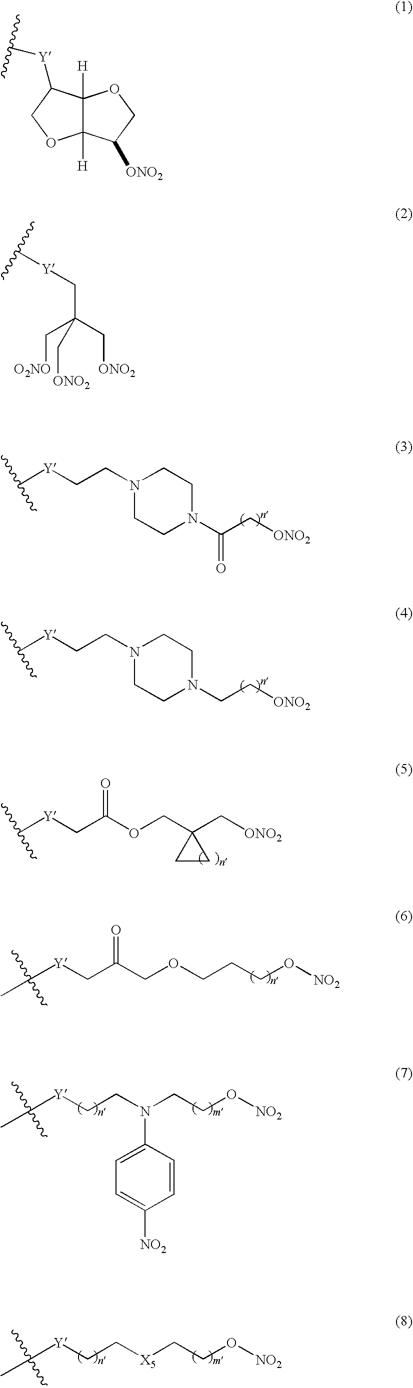 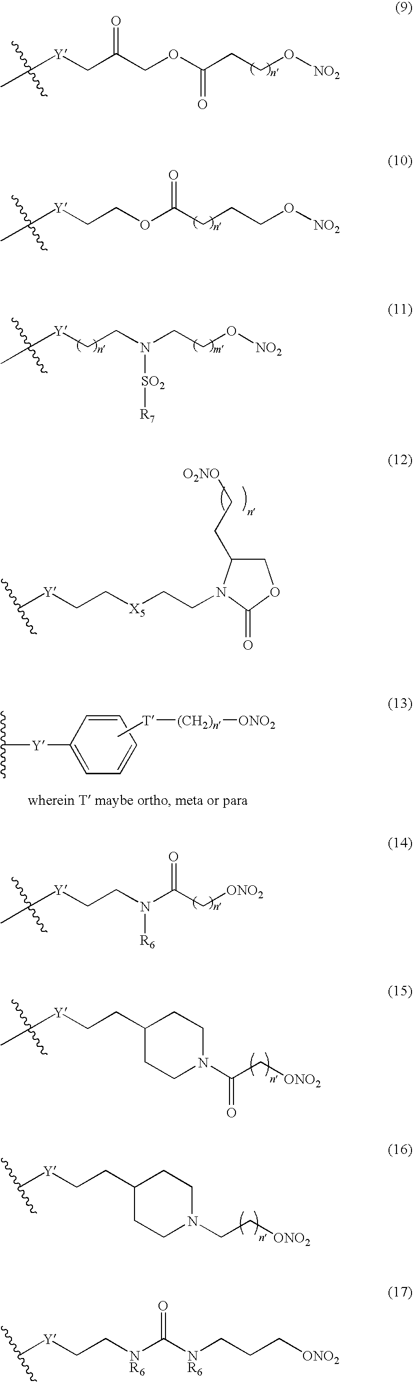 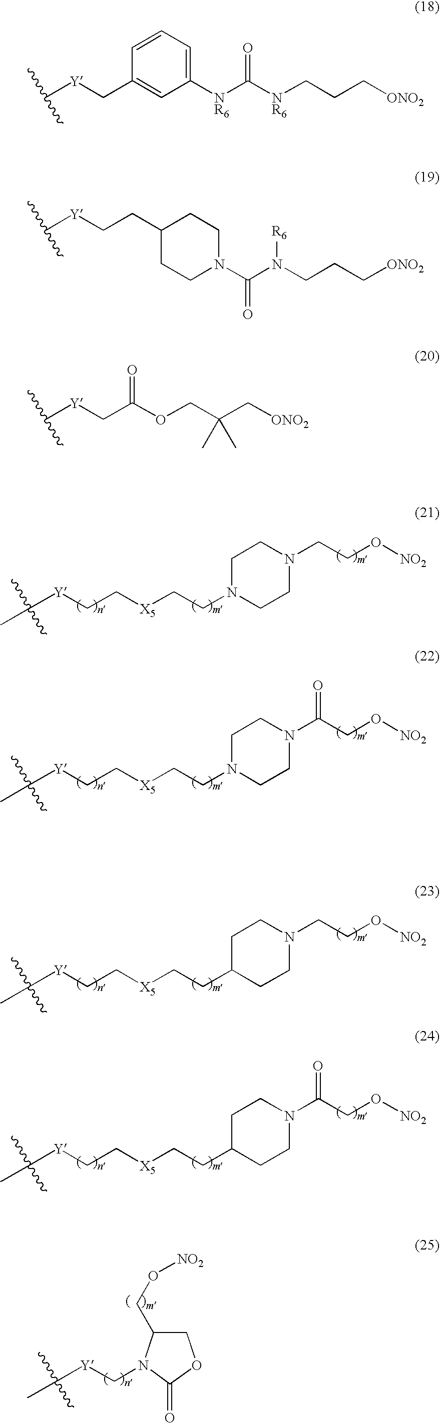 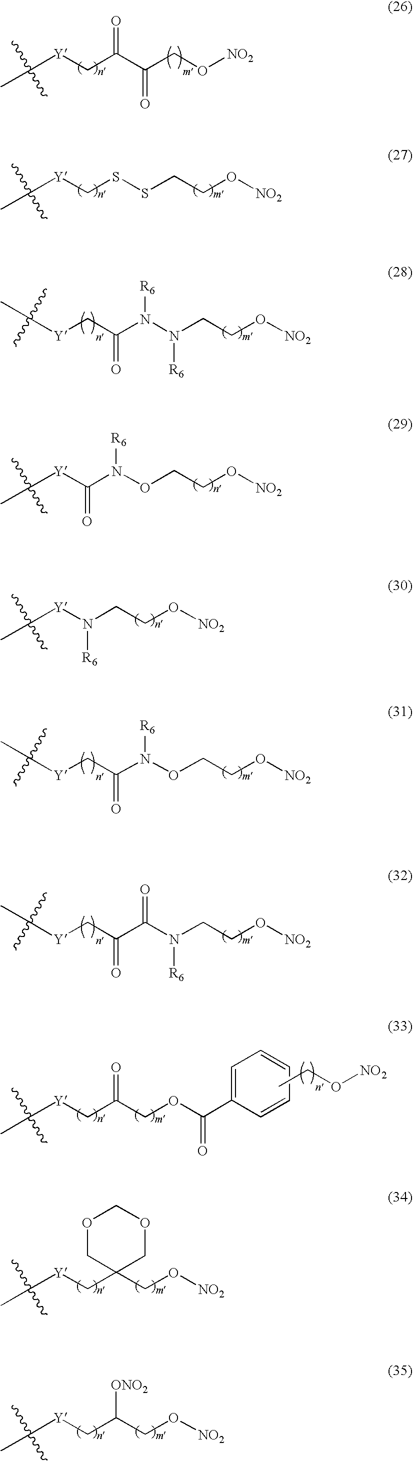 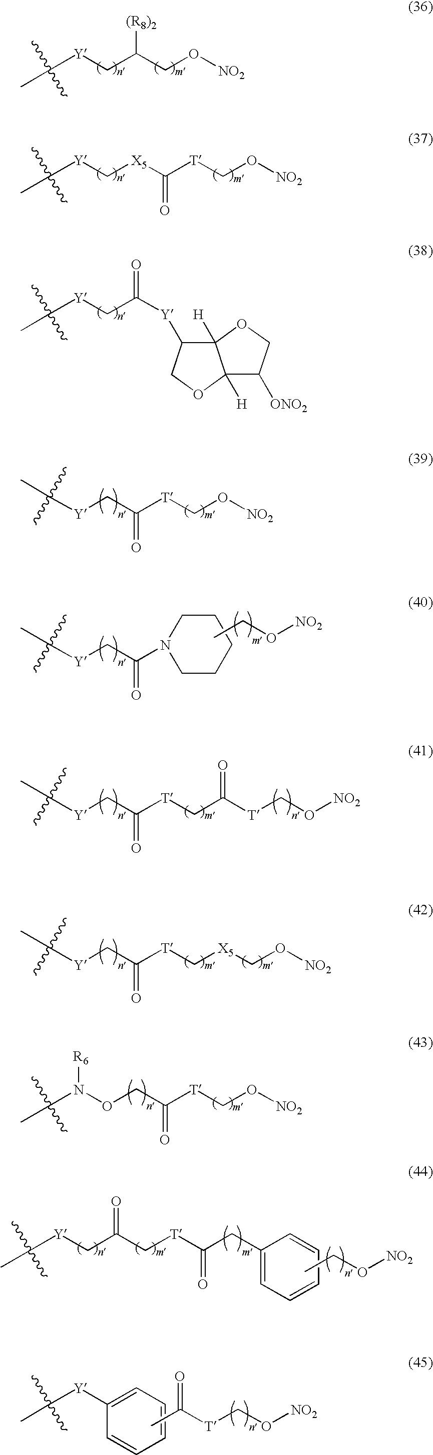 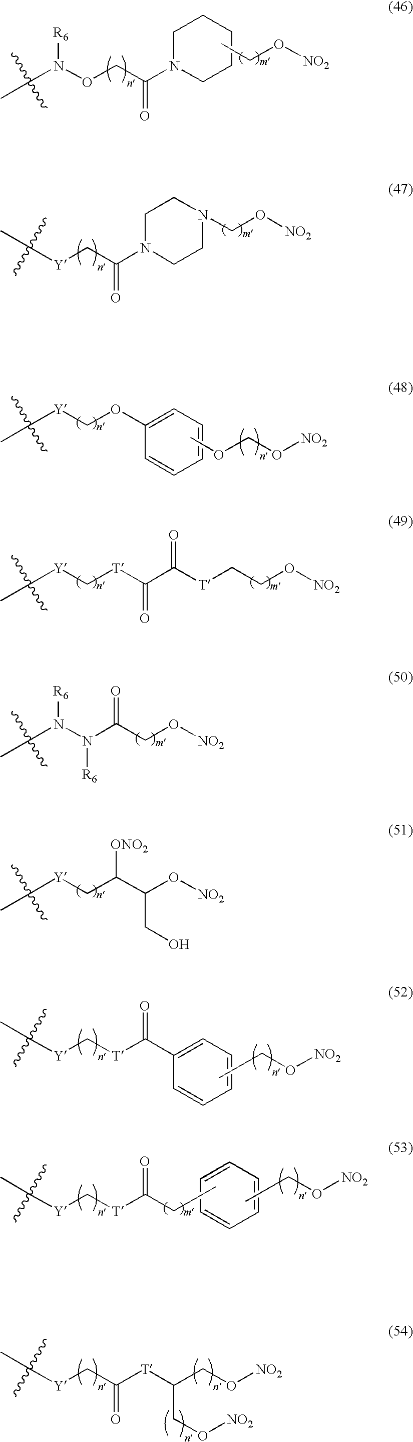 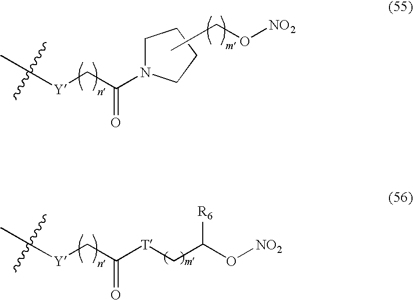
wherein:
Y′ is oxygen or sulfur;
T′ is oxygen, sulfur or NR₆;
X₅ is oxygen, (S(O)ₒ)ₒ or NR₆;
R₆ is a hydrogen, a lower alkyl group, an aryl group;
R₇ is a lower alkyl group or an aryl group;
R₈ at each occurrence is independently is a hydrogen, a hydroxyl group, a lower alkyl group, an aryl group, —NO₂, —CH₂—ONO₂ or —CH₂—OH;
n′ and m′ are each independently an integer from 0 to 10; and
o is as an integer from 0 to 2.

3. A composition comprising the nitrosated antimicrobial compound, the nitrosated adenosine antagonist, the nitrosated LTB4 antagonist, the nitrosated mucoregulator, or the nitrosated purine agonist of claim 1 and a pharmaceutically acceptable carrier.

4. A nitrosated antimicrobial compound, a nitrosated adenosine antagonist, a nitrosated LTB4 antagonist, a nitrosated mucoregulator and a nitrosated purine agonis or a pharmaceutically acceptable slat thereof, having at least one nitrosated carboxylic acid group (—C(O)K), nitrosated hydroxyl group (—OK), nitrosated thiol group (—SK) and/or primary or secondary nitrosated amine group (—NK); wherein the nitrosated antimicrobial compounds is a nitrosated daptomycin, a nitrosated duramycin, a nitrosated nafcillin, a nitrosated tigecycline, a nitrosated PA-1806 or a nitrosated PA-2794; the nitrosated adenosine agonist is a nitrosated CPX; the nitrosated LTB4 antagonist is a nitrosated amelubant; the nitrosated mucoregulators is a nitrosated talniflumate, a nitrosated MSI-2216, a nitrosated ML-03 or a nitrosated INO-4995; the nitrosated purine agonist is a nitrosated P2Y2 agonist or a nitrosated INS-37217, a nitrosated uridine 5′triphosphate, or a nitrosated diquafosol tetrasodium;
wherein:
K is —W′ₐ-E_{b}-(C(Rₑ)(R_{f}))_{p′}-E_{c}-(C(Rₑ)(R_{f}))ₓ—W′_{d}—(C(Rₑ)(R_{f}))_{y}—W′ᵢ-Eⱼ-W′_{g}—(C(Rₑ)(R_{f}))_{z}—U—NO₂;
a, b, c, d, g, i and j are each independently an integer from 0 to 3;
p′, x, y and z are each independently an integer from 0 to 10;
W′ at each occurrence is independently —C(O)—, —C(S)—, -T′-, —(C(Rₑ)(R_{f}))ₕ—, an alkyl group, an aryl group, a heterocyclic ring, an arylheterocyclic ring, or —(CH₂CH₂O)_{q′}—;
E at each occurrence is independently -T′-, an alkyl group, an aryl group, —(C(Rₑ)(R_{f}))ₕ—, a heterocyclic ring, an arylheterocyclic ring, or —(CH₂CH₂O)_{q′}—;
T′ at each occurrence is independently a covalent bond, a carbonyl, an oxygen, —S(O)ₒ— or —N(Rₐ)Rᵢ;
h is an integer form 1 to 10;
q′ is an integer from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, a alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, W′ₕ, —(CH₂)ₒ—U—V₁, or —(C(R_{g})(Rₕ))ₖ—U—V₂, or Rₑ and R_{f} taken together with the carbons to which they are attached form a carbonyl, a methanthial, a heterocyclic ring, a cycloalkyl group, an aryl group, an oxime, a hydrazone or a bridged cycloalkyl group;
R_{g} and Rₕ at each occurrence are independently Rₑ;
k is an integer from 1 to 3;
U at each occurrence is independently a covalent bond, a carbonyl, an oxygen, —S(O)O— or —N(Rₐ)Rᵢ;
V₁ is —NO or —NO₂;
o is an integer from 0 to 2;
Rₐ is a lone pair of electrons, a hydrogen or an alkyl group;
Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U—V₁)(Rₑ)(R_{f}), a bond to an adjacent atom creating a double bond to that atom, —(N₂O₂—)⁻.M⁺, wherein M⁺ is an organic or inorganic cation.

5. A composition comprising the nitrosated antimicrobial compound, the nitrosated adenosine antagonist, the nitrosated LTB4 antagonist, the nitrosated mucoregulator, or the nitrosated purine agonist of claim 4 and a pharmaceutically acceptable carrier.

6. A nitrosylated antimicrobial compound, a nitrosylated adenosine antagonist, a nitrosylated LTB4 antagonist, a nitrosylated mucoregulator, a nitrosylated purine agonist, or a pharmaceutically acceptable salt thereof having at least one nitrosylated carboxylic acid group (—C(O)K₁), nitrosylated hydroxyl group (—OK₁), nitrosylated thiol group (—SK₁) and/or primary or secondary nitrosylated amine group (—NK₁);
wherein
K₁ is —W′ₐ-E_{b}-(C(Rₑ)(R_{f}))_{p′}-E_{c}-(C(Rₑ)(R_{f}))ₓ—W′_{d}—(C(Rₑ)(R_{f}))_{y}—W′ᵢ-Eⱼ-W′_{g}—(C(Rₑ)(R_{f}))_{z}—U—NO; and
a, b, C, d, g, i, j, p′, x, y, z, W′, E, Rₑ, R_{f} and U are as defined herein.

7. A composition comprising the nitrosated antimicrobial compound, the nitrosated adenosine antagonist, the nitrosated LTB4 antagonist, the nitrosated mucoregulator, or the nitrosated purine agonist of claim 6 and a pharmaceutically acceptable carrier.

8. The composition of claim 3, 5 or 7, wherein the antimicrobial compound is amikacin, azetreonam, azithromycin, colistin, duramycin, gentamycin, tigecycline, tobramycin, vancomycin, PA-1806 and PA-2794.

9. A method for treating a bacterial infection; treating a viral infection; treating a fungal infection; and/or treating a lesions in a patient in need thereof comprising administering to the patient a therapeutically effective amount of the composition of claim 3, 5 or 7.

10. The method of claim 9, wherein the bacterial infection is a pulmonary infection selected from the group consisting of an endobronchial infection, cystic fibrosis, bronchiectasis, pneumonia, tuberculosis, emphysema, AIDS, pneumoccal meningitis, bacteremia, otitis media, chronic obstructive pulmonary disease, sinus congestion, common cold, septicemia; a gastrointestinal infection selected from the group consisting of chronic gastritis, a gastric ulcer, a duodenal ulcer, Helicobacter pylori, a gastric malignant lymphoma, gastroenteritis, diarrhea, dysentery, an inflammatory bowel disease, Crohn's disease, an ulcerative colitisan; an infection resulting from E. coli; and/or an infection of an eye, an ear or a nose.

11. The method of claim 10, wherein the bacterial infection is cystic fibrosis.

12. The composition of claim 3, 5 or 7, further comprising (i) at least one therapeutic agent; (ii) at least one nitric oxide donor compound; or (iii) at least one therapeutic agent and at least one nitric oxide donor compound.

13. The composition of claim 12, wherein the therapeutic agent is an aldosterone antagonist, an alpha-adrenergic receptor antagonist, a β-adrenergic agonist, an anti-allergic compound, an antidiabetic compound, an anti-hyperlipidemic drug, an antitussive compound, an angiotensin II antagonist, an angiotensin-converting enzyme inhibitor, an antioxidant, an antithrombotic and vasodilator drug, a β-adrenergic antagonists, a bronchodilator, a calcium channel blocker, a diuretic, an endothelin antagonist, an expectorant, a hydralazine compound, a H₂ receptor antagonist, a neutral endopeptidase inhibitor, a nonsteroidal antiinflammatory compound, a phosphodiesterase inhibitor, a potassium channel blocker, a platelet reducing agent, a proton pump inhibitor, a renin inhibitor, a selective cyclooxygenase-2 (COX-2) inhibitor, a steroid, or a combination of two or more thereof.

14. The composition of claim 13, wherein the nitric oxide donor compound is selected from the group consisting of a S-nitrosothiol, a nitrite, a nitrate, a S-nitrothiol, a sydnonimine, a NONOate, a N-nitrosoamine, a N-hydroxyl nitrosamine, a nitrosimine, a diazetine dioxide, an oxatriazole 5-imine, an oxime, a hydroxylamine, a N-hydroxyguanidine, a hydroxyurea or a furoxan.

15. A method for treating a bacterial infection; treating a viral infection; treating a fungal infection; and/or treating a lesions in a patient in need thereof comprising administering to the patient a therapeutically effective amount of the composition of claim 12.

16. A kit comprising at least one compound of claim 1, claim 4 or claim 6.

17. The kit of claim 16, further comprising further comprising (i) at least one therapeutic agent; (ii) at least one nitric oxide donor compound; or (iii) at least one therapeutic agent and at least one nitric oxide donor compound.

18. The kit of claim 17, wherein the (i) at least one therapeutic agent; (ii) at least one nitric oxide donor compound; or (iii) at least one therapeutic agent and at least one nitric oxide donor compound are in the form of separate components in the kit.
